(19) United States
(12) Patent Application Publication
(10) Pub. No.: US 2025/0177446 A1
(43) Pub. Date: Jun. 5, 2025

(54) METABOLICALLY MODIFIED T-CELLS, COMPOSITIONS COMPRISING SAME AND USES THEREOF

(71) Applicant: BAR ILAN UNIVERSITY, Ramat Gan (IL)

(72) Inventors: Cyrille Joseph COHEN, Petach-Tikva (IL); Raphaelle TOLEDANO, Tel Aviv (IL); Tilda BARLIYA, Rosh HaAyin (IL)

(21) Appl. No.: 18/845,389

(22) PCT Filed: Mar. 9, 2023
(86) PCT No.: PCT/IL2023/050248

§ 371 Date: Sep. 9, 2024

Related U.S. Application Data
(60) Provisional application No. 63/317,967, filed on Mar. 9, 2022.

(51) Int. Cl. A61K 35/17; A61K 40/11; A61K 40/31; A61K 40/32; A61K 40/42; A61P 35/00; C07K 14/725; C12N 5/0783; C12N 9/12

(52) U.S. Cl. CPC A61K 35/17; A61K 40/11; A61K 40/31; A61K 40/32; A61K 40/4211; A61K 40/4272; A61P 35/00; C07K 14/7051; C12N 5/0636; C12N 9/1205; A61K 2239/13; A61K 2239/17; A61K 2239/21; C07K 2319/03; C12N 2510/00; C12Y 207/01001; C12Y 207/01011; C12Y 207/0104

(57) ABSTRACT

The present disclosure relates to genetically engineered hematopoietic cell/s, specifically, lymphocytes, and more specifically, cells of the T cell lineage or a cell population comprising at least one of the cell/s. The disclosed cells comprises and/or expresses at least one nucleic acid sequence encoding at least one molecule involved directly or indirectly in at least one metabolic pathway. The present disclosure provides compositions, methods and uses of the engineered cells.

### FIELD OF THE INVENTION

The present disclosure relates to immunotherapy. More specifically, the present disclosure relates to metabolically modified, specifically, genetically engineered hematopoietic cells, specifically, T-Cells, compositions and methods thereof for the treatment of immune-related disorders, specifically cancer.

### BACKGROUND ART

References considered to be relevant as background to the presently disclosed subject matter are listed below:
[1] Rivera G O. R, et al., Frontier in Immunology, 2021, Vol 12. Pages 1-17;
[2] Mathupala S P et al, Oncogene, 2006 7; 25(34):4777-86. Acknowledgement of the above references herein is not to be inferred as meaning that these are in any way relevant to the patentability of the presently disclosed subject matter.

### BACKGROUND OF THE INVENTION

Metabolic pathways like glycolysis and oxidative phosphorylation are indispensable for cell survival [Lim, A. R., et al. ELife, 9, 1-13 (2020)]. Glucose is a major source of energy in mammalian cells, generating ATP through glycolysis and oxidative phosphorylation.

It is also a precursor of amino acids, nucleotides and lipids [Li, B., & Pan, F. (Eds.). Advances in Experimental Medicine and Biology 1011:1-85 (2017)].

In glycolysis, glucose (C₆H₁₂O₆) is converted into pyruvic acid (CH₃COCO₂H) and the free energy released in this process is used to form the high-energy molecules adenosine triphosphate (ATP) and reduced nicotinamide adenine dinucleotide (NADH). In each step of the glycolysis, important enzymes catalyze reactions. The main reactions in the glycolysis pathway are catalyzed by hexokinase, phosphofructokinase, and pyruvate kinase; hence, these enzymes have a regulatory role.

They are considered as ‘control sites’ [Berg J M., et al., Biochemistry. 5th edition. New York: W H Freeman; Section 16.1 and Section 16.2 (2002)].

The enzyme Hexokinase (HK) catalyzes the first step of the glycolysis pathway. It mediates the irreversible first step of the glycolytic pathway by catalyzing the phosphorylation of D-glucose to D-glucose 6-phosphate with concomitant de-phosphorylation of ATP.

Phosphofructokinase (PFK) is another important control element in the glycolytic pathway. It catalyzes the third step of the glycolysis, which is ATP-dependent, in which fructose-6-phosphate is converted into fructose-1,6-phosphate.

Pyruvate kinase (PKM) plays a pivotal role in regulating cell metabolism, by catalyzing the conversion of Phosphoenolpyruvate (PEP) to Pyruvate.

The transport of glucose across the plasma membrane into the cytosol is a rate-limiting step in glucose metabolism and is mediated by a family of glucose transporters (GLUTs). GLUT3 are present in nearly all mammalian cells, have a key role in glucose uptake in the cell. Cellular metabolic pathways, including glycolysis, provide not only energy but also a variety of metabolic intermediates that can regulate differentiation and function [Rangel Rivera G O, et al., Front Immunol. 12:645242 (2021); Moritz Rapp et al., Semin Immunopathol. (2018); 40(4): 343-355]. More specifically, the regulation of metabolic reprogramming during T cell immune responses involves a complex network of cytokines, enzymes, membrane transporters and transcription factors [Marek Sinkora and John E. Butler. Dev Comp Immunol. 58:1-17 (2016)].

Rivera G O. R, et al., [1] review the fundamental metabolic requirements for T cells to survive, proliferate and mount antigen-specific responses in the context of effector and memory responses and the metabolic manipulations previously performed.

T cells use different metabolic pathways based on their differentiation and memory status. Resting T cells largely depended on oxidative phosphorylation (OXPHOS) of glucose to survive. Upon T cell stimulation, T cells undergo protein and transcriptional changes in metabolism that allow the sustained activity of glycolysis and other amino acid uptake and usage. Glycolysis by products in effector T cells mediate changes that help sustain effector cytokine release and cytolytic function. Effector T cells that clear antigen either die or contract to form memory T cells. Compared to effectors, memory T cells possess an enhanced metabolic profile dependent on mitochondrial biogenesis, mitochondrial fusion and reliance on fatty acid oxidation.

As T cell survival is often impaired in patients with cancer and chronic infectious disease, it is necessary to have an effective metabolic capacity for a productive immune response. Furthermore, harsh tumor microenvironment manipulates T cell metabolism to impair effector functions. Emerging reports reveal that tumors and activated T cells share common metabolic programs to survive, thus setting the stage for a continuous battle (or tug of war) for nutrients. Tumors consume key metabolites in the host to survive, thus robbing T cells of these nutrients to function and thrive. T cells are often deprived of basic building blocks for energy in the tumor, including glucose and amino acids needed to proliferate or produce cytotoxic molecules against tumors.

Accordingly, there is a need to enhance T cell mediated immunity to tumors and to improve T cell-based immunotherapies for cancer patients.

### SUMMARY OF THE INVENTION

A first aspect of the present disclosure relates to at least one genetically engineered hematopoietic cell/s or a cell population comprising at least one of the cell/s. More specifically, the genetically engineered cell/s in accordance with the present disclosure comprises and/or expresses at least one nucleic acid sequence encoding at least one molecule involved directly or indirectly in at least one metabolic pathway. In some embodiments, the expressed nucleic acid sequence may be exogenously expressed nucleic acid sequence/s. In some embodiments, the genetically engineered cell/s of the present disclosure may optionally further express, or may be engineered to further express, at least one receptor molecule. In some embodiments, the genetically engineered hematopoietic cells may be any lymphocyte cells. In yet some further embodiments, the genetically engineered lymphocytes of the present disclosure may be genetically engineered cells of the T cell lineage.

A further aspect of the present disclosure relates to a composition comprising at least one of: In some embodiments the disclosed compositions may comprise (a), at least one genetically engineered hematopoietic cell/s or a cell population comprising at least one of the genetically engineered cell/s. The genetically engineered cell/s of the disclosed compositions, comprise and/or expresses (e.g., exogenously) at least one nucleic acid sequence encoding at least one molecule involved directly or indirectly in at least one metabolic pathway. The cells of the disclose composition optionally further expresses at least one receptor molecule. In yet some alternative or additional embodiments, the disclosed composition may further comprise and/or alternatively comprise (b), at least one nucleic acid sequence encoding the at least one molecule involved directly or indirectly in at least one metabolic pathway, or any cassette, vector and/or gene editing system comprising the nucleic acid sequence. In some embodiments, the composition further comprises at least one of pharmaceutically acceptable carrier/s, diluent/s, excipient/s and additive/s. In some embodiments, the genetically engineered hematopoietic cells may be any lymphocyte cells. In yet some further embodiments, the genetically engineered lymphocytes of the present disclosure may be genetically engineered cells of the T cell lineage.

A further aspect of the present disclosure relates to a method for treating, preventing, ameliorating, inhibiting or delaying the onset of a pathologic disorder in a mammalian subject. The methods disclosed herein comprise the step of administering to the subject an effective amount of at least one of: (a), at least one genetically engineered hematopoietic cell/s or a cell population comprising at least one of the genetically engineered cell/s. The genetically engineered cell/s used by the disclosed methods, comprises and/or expresses, specifically, exogenously, at least one nucleic acid sequence encoding at least one molecule involved directly or indirectly in at least one metabolic pathway. The cell/s used by the disclosed methods may optionally further expresses (either exogenously or endogenously, at least one receptor molecule. In yet some further embodiments, the methods disclosed herein may further, or alternatively administer (b), at least one nucleic acid sequence encoding the at least one molecule involved directly or indirectly in at least one metabolic pathway, or any cassette, vector and/or gene editing system comprising the nucleic acid sequence. Still further, the disclosed methods may further, or alternatively administer (c), a composition comprising the genetically engineered cell/s of (a) and/or the nucleic acid sequence of (b). In some embodiments, the genetically engineered hematopoietic cells may be any lymphocyte cells. In yet some further embodiments, the genetically engineered lymphocytes of the present disclosure may be genetically engineered cells of the T cell lineage.

A further aspect of the present disclosure relates to a therapeutically effective amount of at least one of: (a), at least one genetically engineered hematopoietic cell/s or a cell population comprising at least one of the genetically engineered cell/s. It should be noted that the genetically engineered cell/s comprises and/or expresses (e.g., exogenously), at least one nucleic acid sequence encoding at least one molecule involved directly or indirectly in at least one metabolic pathway. Still further, in some embodiments of the disclosed use, the cell/s optionally further expresses at least one receptor molecule; (b), at least one nucleic acid sequence encoding the at least one molecule involved directly or indirectly in at least one metabolic pathway, or any cassette, vector and/or gene editing system comprising the nucleic acid sequence; and/or (c), a composition comprising said genetically engineered cell/s of (a) and/or the nucleic acid sequence of (b), for use in a method for treating, preventing, ameliorating, inhibiting or delaying the onset of a pathologic disorder in a mammalian subject. In some embodiments, the genetically engineered hematopoietic cells may be any lymphocyte cells. In yet some further embodiments, the genetically engineered lymphocytes of the present disclosure may be genetically engineered cells of the T cell lineage.

A further aspect provided by the present disclosure relates to a method for improving activity and/or survival of at least one hematopoietic cell. The method disclosed herein may comprise the step of contacting at least one cell with an effective amount of at least one nucleic acid sequence encoding at least one molecule involved directly or indirectly in at least one metabolic pathway, or any cassette, vector and/or gene editing system comprising the nucleic acid sequence, the cell optionally further expresses at least one receptor molecule. In some embodiments, the genetically engineered hematopoietic cells may be any lymphocyte cells. In yet some further embodiments, the genetically engineered lymphocytes of the present disclosure may be genetically engineered cells of the T cell lineage.

A further aspect of the present disclosure relates to a therapeutically effective amount of at least one of: (a), at least one genetically engineered hematopoietic cell/s or a cell population comprising at least one of the genetically engineered cell/s. The genetically engineered cell/s used herein, comprise and/or expresses (e.g., exogenously) at least one nucleic acid sequence encoding at least one molecule involved directly or indirectly in at least one metabolic pathway. These cells may optionally further express at least one receptor molecule; (b), at least one nucleic acid sequence encoding the at least one molecule involved directly or indirectly in at least one metabolic pathway, or any cassette, vector and/or gene editing system comprising said nucleic acid sequence; and (c), at least one composition comprising the genetically engineered cell/s of (a) and/or the nucleic acid sequence of (b), for use in a method for improving activity and/or survival of at least one hematopoietic cell of. In some embodiments, the genetically engineered hematopoietic cells may be any lymphocyte cells. In yet some further embodiments, the genetically engineered lymphocytes of the present disclosure may be genetically engineered cells of the T cell lineage.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. Scheme depicting the main actual protagonists The figure illustrates the glycolysis pathway in T cells.

FIG. 2. Delivery of glucose and ATP to Hexokinase (HK) II

The figure illustrates hexokinase and mechanism of action of this enzyme. The scheme is obtained from [2].

FIG. 3A-3E. Expression levels of glucose metabolism related genes in engineered T cells

FIG. 3A. HK2 and NGFR expression in engineered T-Cells. Flow Cytometry analysis of human PBLs transduced with retroviral vector to express HK2 using specific antibodies for HK2. The percentage of positive cells and mean fluorescence intensity (MFI, in brackets) are shown. Results are representative of 16 independent experiments with 13 different donors.

FIG. 3B. PFK and NGFR expression in engineered T-Cells. Flow Cytometry analysis of human PBLs transduced with retroviral vector to express PFK using specific antibodies for PFK. The percentage of positive cells and mean fluorescence intensity (MFI in brackets) are shown. Results are representative of 16 independent experiments with 13 different donors.

FIG. 3C. Glut3 and NGFR expression in engineered T-Cells. Flow Cytometry analysis of human PBLs transduced with retroviral vectors to express Glut3 and a truncated version of NGFR (GLUT3-I-NGFR) or NGFR, using a specific antibody for NGFR. The truncated NGFR gene is a reporter gene, cloned into the vector with Glut3 (separate with an IRES sequence). The percentage of positive cells and mean fluorescence intensity (MFI in brackets) are shown. Results are representative of 25 independent experiments with 20 different donors.

FIG. 3D. PFK, Glut3 and NGFR expression in engineered T-Cells. How Cytometry analysis of human PBLs transduced with retroviral vectors to express PFK, Glut3 and a truncated version of NGFR, using a specific antibody for PFK. The percentage of positive cells and mean fluorescence intensity (MFI in brackets) are shown. Results are representative of 8 independent experiments with 6 different donors.

FIG. 3E. A real-time PCR for the expression of PFK or GLUT3 in T-cells transduced with PFK-I-GLUT3 relative to control cells (untransduced cells). Results presented as mean+SEM of 3 independent experiments performed with 3 different donors, p-value was calculated using a Student's paired t-test one-tail.

FIG. 4A-4B. Expression of HK2 in T cells highly improve cytokines secretion FIG. 4A

FIG. 4A(i)-4A(iii). ELISA analysis of IL-2 (FIG. 4Ai), TNFα (FIG. 4Aii), and IFNγ (FIG. 4Aiii) secretion in supernatant collected from PBLs T-cells engineered to co-express TCR specific for MART-1 (termed F4) with either NGFR (control) or HK2 and co-cultured with negative (A375) or positive target cells (SKMEL23, 888A2). NoT indicates—cells cultured with no Target cells (both, NGFR+F4 and HK2+ are used). Results are mean+SEM of independent experiments performed as follows and normalized to NGFR (control): for IL-2, 3 experiments with 3 different donors; for IFNγ, 5 experiments with 5 donors; for TNFα, 7 experiments with 6 donors p-value was calculated using a Student's paired t-test one-tail.

FIG. 4B(i)-4B(iii). ELISA analysis for IFNγ (FIG. 4Bi), IL-2 (FIG. 4Bii), and TNFα (FIG. 4Biii) secretion in supernatant collected from PBLs T-cells engineered to express NGFR (control) or HK2 and unstimulated or stimulated with the stimulating antibody OKT3. Results are mean+SEM of independent experiments performed as follows and normalized to NGFR (control): for IL-2, 5 experiments with 5 donors; for IFNγ, 6 experiments with 5 donors; for TNFα, 11 experiments with 9 donors p-value was calculated using a Student's paired t-test one-tail.

FIG. 5A-5C. PFK Engineered T cells highly improve cytokines secretion FIG. 5A(i)-5A(iii). ELISA analysis for TNFα (FIG. 5Ai), IFNγ (FIG. 5Aii) and IL-2 (FIG. 5Aiii) secretion in supernatant collected from PBLs T-cells engineered to express NGFR (control) or PFK and stimulated or unstimulated with OKT3. Results are the mean+SEM of 3 independent experiments performed with 3 different donors and normalized to NGFR (control). p-value was calculated using a Student's paired t-test one-tail.

FIG. 5B(i)-5B(iii). ELISA analysis of TNFα (FIG. 5Bi), IFNγ (FIG. 5Bii) and IL-2 (FIG. 5Biii) secretion in supernatant collected from PBLs T-cells engineered to co-express TCR specific for MART-1 (termed F5) with either NGFR (control) or PFK and co-cultured with positive target cells (624.38, SKMEL23, 888A2) or negative target cells (A375). No-T represent cells cultured with no target cells. Results are the mean+SEM of 3 independent experiments performed with 3 different donors and normalized to NGFR (control). p-value was calculated using a Student's paired t-test one-tail.

FIG. 5C(i)-5C(iii): ELISA analysis for TNFα (FIG. 5Ci), IFNγ (FIG. 5Cii) and IL-2 (FIG. 5Ciii) secretion in supernatant collected from PBLs T-cells expressing a chimeric antigen receptor (CAR) consisting of an anti-CD19 scFv (single chain variable fragment) coupled to the costimulatory signaling domain CD28 transduced to express either NGFR (control) or PFK and co-cultured with negative (K562) or positive target cells (K562-CD19). Results are the mean+SEM of 3 independent experiments performed with 3 different donors and normalized to NGFR (control). p-value was calculated using a Student's paired t-test one-tail.

FIG. 6A-6C. Glut3 Engineered T cells highly improve cytokines secretion

FIG. 6A. ELISA analysis for IL-2 secretion in supernatant collected from PBLs T-cells engineered to express NGFR (control) or GLUT3 and NGFR (NGFR-I-GLUT3) and unstimulated or stimulated with OKT3. Results are the mean+SEM of 5 independent experiments performed with 3 different donors and normalized to NGFR (control). p-value was calculated using a Student's paired t-test one-tail.

FIG. 6B. ELISA analysis of IFNγ secretion in supernatant collected from PBLs T-cells engineered to co-express TCR specific for MART-1 (termed F5) with either NGFR(control) or GLUT3 and NGFR (GLUT3-I-NGFR), and co-cultured with negative (A375) or positive target cells (SKMEL23, 888A2). NoT represents T cells without target cells. Results are the mean+SEM of at least 7 independent experiments performed with at least 5 different donors and normalized to NGFR (control). p-value was calculated using a Student's paired t-test one-tail.

FIG. 6C. ELISA analysis of TNFα secretion in supernatant collected from PBLs T-cells engineered to co-express TCR specific for MART-1 (termed F5) with either GLUT3 (GLUT3) or an empty vector (control), and co-cultured with negative (A375) or positive target cells (SKMEL23, 888A2). No Target represents T cells without target cells. Results are the mean+SEM of 6 independent experiments performed with 4 different donors and normalized to empty vector (control). p-value was calculated using a Student's paired t-test one-tail.

FIG. 7A-7C. Engineered T cells co-expressing PFK and Glut3 highly improve cytokines secretion

FIG. 7A-7B. ELISA analysis for IL-2 secretion (FIG. 7A) or IFNγ secretion (FIG. 7B) in supernatant collected from PBLs T-cells engineered to express NGFR (control) or PFK and GLUT3 (PFK-I-GLUT3) and unstimulated or stimulated with OKT3. Results are the mean+SEM of 4 independent experiments performed with 3 different donors and normalized to NGFR (control). p-value was calculated using a Student's paired t-test one-tail.

FIG. 7C(i)-7C(iii). ELISA analysis of TNFα (FIG. 7Ci), IFNγ (FIG. 7Cii) and IL-2 (FIG. 7Ciii) secretion in supernatant collected from PBLs T-cells engineered to co-express TCR specific for MART-1 with either NGFR (control) or PFK and GLUT3 (PFK-I-GLUT3), and co-cultured with negative (A375) or positive target cells (624.38, SKMEL23, 888A2). The results are the mean+SEM of 5 independent experiments performed with 5 different donors and normalized to NGFR (control).

FIG. 8A-8C. Upregulation of T-cells activation markers in HK2 engineered T cells

FIG. 8A-8B: HK2 and NGFR transduced T-cells were cultured after OKT3 activation and the expression of CD137 marker (FIG. 8A) or CD69 marker (FIG. 8B) was measured by FACS, gated on the CD8+ population. The results are the mean+SEM of 5 independent experiments performed with 4 different donors and normalized to NGFR (control). The difference in the expression levels of the indicated markers between the HK2 engineered T-cells and the control cells (NGFR) is statistically significant in Student's paired t-test.

FIG. 8C: HK2 and NGFR transduced T-cells were cultured after OKT3 activation and the expression CD25 marker was measured by FACS, gated on the CD4+ population. The results are 6 independent experiments performed with 5 different donors. The difference in the expression levels of the indicated markers between the HK2 engineered T-cells and the control cells (NGFR) is statistically significant in Student's paired t-test.

FIG. 9A-9C. Upregulation of T-cells activation markers in PFK engineered T cells FIG. 9A-9B. PFK and NGFR transduced T-cells were cultured after OKT3 activation and the expression of CD137 marker (FIG. 9A), or CD69 marker (FIG. 9B) was measured by FACS, gated on the CD8+ population. The results are 5 independent experiments performed with 4 different donors. The difference in the expression levels of the indicated markers between the PFK engineered T-cells and the control cells (NGFR) is statistically significant in Student's paired t-test.

FIG. 9C. PFK and NGFR transduced T-cells were cultured after OKT3 activation and the expression of CD25 marker was measured by FACS, gated on the CD4+ population. The results are 5 independent experiments performed with 4 different donors. The difference in the expression levels of the indicated markers between the PFK engineered T-cells and the control cells (NGFR) is statistically significant in Student's paired t-test.

FIG. 10A-10B. Upregulation of T-cells activation markers in Glut3 Engineered T cells

FIG. 10A. GLUT3 and NGFR transduced T-cells were cultured after OKT3 activation and the expression of CD137 marker was measured by FACS, gated on the CD8+ population. The results are 6 independent experiments performed with 4 different donors. The difference in the expression levels of the indicated markers between GLUT3 engineered T-cells and the control cells (NGFR) is statistically significant in Student's paired t-test.

FIG. 10B. GLUT3 and NGFR transduced T-cells were cultured after OKT3 activation and the expression of CD69 marker was measured by FACS, gated on the CD8+ population. The results are 5 independent experiments performed with 4 different donors. The difference in the expression levels of the indicated markers between GLUT3 engineered T-cells and the control cells (NGFR) is statistically significant in Student's paired t-test.

FIG. 11A-11C. Upregulation of T-cells activation markers in engineered T cells co-expressing PFK and GLUT3

FIG. 11A-11B. PFK-I-GLUT3 and NGFR transduced T-cells were cultured after OKT3 activation and the CD137 marker (FIG. 11A) or, CD69 marker (FIG. 11B) was measured by FACS, gated on the CD8+ population. The results are 6 independent experiments performed with 3 different donors. The difference in the expression levels of the indicated markers between PFK-I-GLUT3 engineered T-cells and the control cells (NGFR) is statistically significant in Student's paired t-test.

FIG. 11C. PFK-I-GLUT3 and NGFR transduced T-cells were cultured after OKT3 activation and the CD25 marker was measured by FACS, gated on the CD4+ population. The results are 6 independent experiments performed with 3 different donors. The difference in the expression levels of the indicated markers between PFK-I-GLUT3 engineered T-cells and the control cells (NGFR) is statistically significant in Student's paired t-test.

FIG. 12A-12D. HK2 mediates anti-tumor cytotoxic

FIG. 12A. HK2 TCR F4 transduced cells and TCR F4 control cells (Effector (E) cells) were co-cultured with positive 888A2 target (T) cells at 5:1 E:T ratio. After 12 h, images were analyzed using the IncuCyte S3 Live-Cell Analysis system.

FIG. 12B. HK2 TCR F4 transduced cells and TCR F4 control cells (E cells) were co-cultured with negative A375 target cells (T cells) at 5:1 E:T ratio. After 12 h, images were analyzed using the IncuCyte S3 Live-Cell Analysis system.

FIG. 12C. HK2 TCR F4 transduced cells and TCR F4 control cells (E cells) were co-cultured with positive 888A2 target cells (T cells) at 2:1 E:T ratio. After 12 h, images were analyzed using the IncuCyte S3 Live-Cell Analysis system. The results are presented as mean+SEM of at least 3 independent experiments with 3 different donors and the difference between the HK2 and the control group was found statistically significant as indicated (using a Student's paired t-test).

FIG. 12D. HK2 TCR F4 transduced cells and TCR F4 control cells (E cells) were co-cultured with negative A375 target cells (T cells) at 2:1 E:T ratio. After 12 h, images were analyzed using the IncuCyte S3 Live-Cell Analysis system.

Cytotoxicity was calculated based on the percent of mCherry+ population.

FIG. 13A-13B. Downregulation of exhaustion markers in modified T cells

FIG. 13A. FACS analysis of TIM3 expression levels in PBLs transduced with HK2 or NGFR and cultured in glucose containing or deficient medium, using antibodies specific for TIM3. The percentage of positive cells is shown. The results are the mean+SEM of 4 independent experiments with 2 different donors. The difference in TIM3 expression between HK2 cells and NGFR control cells is significant (p-value 0.002, using a Student's paired t-test).

FIG. 13B. FACS analysis of TIM3 and LAG3 expression levels in T-cells expressing CD19-specific 4-1BB based CAR transduced with PFK-I-GLUT3 or NGFR and cultured in glucose-free medium for 2 hours using antibodies specific for TIM3 or LAG3. The results are the mean+SEM of 3 independent experiments with 2 different donors. The difference in TIM3 and LAG3 expression levels between PFK-IRES-GLUT3 cells and NGFR control cells is significant (p-value=0.036 and p=0.039, using a Student's paired t-test).

FIG. 14A-14D. Increased glycolysis and glycolytic capacity in engineered T cells FIG. 14A. Glycolysis rate in HK2 transduced T cells and control NGFR cells was measured by seahorse, showing representative ECAR (±SD) normalized to cell count measured. The results are mean±SD (of 8 replicates) representative of 3 experiments.

FIG. 14B. Histogram of FIG. 14A, representing glycolysis, glycolysis capacity and glycolysis reserve calculated based on the changes in extracellular acidification. The results are presented as mean+SEM of at least 3 independent experiments with 3 different donors and the difference between the HK2 and the control group was found statistically significant as indicated (using a Student's paired t-test).

FIG. 14C. Glycolysis rate in PFK-I-GLUT3 transduced T cells and control NGFR cells was measured by seahorse, showing representative ECAR (±SD) normalized to cell count measured. The results are mean±SD (of 8 replicates) representative of 6 experiments.

FIG. 14D. Histogram of A, representing glycolysis, glycolysis capacity and glycolysis reserve calculated based on the changes in extracellular acidification rate (n=6).

The results are presented as mean+SEM of at least 6 independent experiments with 4 different donors and the difference between the PFK-I-GLUT3 and the control group was found statistically significant as indicated (using a Student's paired t-test).

FIG. 15A-15D. Increased glucose uptake and ATP production in engineered T cells

FIG. 15A. Glucose uptake was measured by flow cytometry using 2-NBDG fluorescent, gated on NGFR+ positive population. The MFI of the negative control cells (presented in white), the NGFR transduced T-cells (presented in grey) and the GLUT3-I-NGFR transduced T-cells (presented in blue) are shown (a representative FACS).

FIG. 15B. Graph of glucose uptake. These results are the mean+SEM of 4 independent experiments with 3 different donors. A significant difference in glucose uptake between NGFR transduced T-cells (control) and GLUT3 transduced T cells was observed (p-value=0.028, using a Student's paired t-test).

FIG. 15C. ATP levels in PBLs transduced with HK2 or NGFR (control) was measured using ATP reagent Cell titerGlo 2.0 assay. ATP levels were normalized to control (NGFR). The results are the mean+SEM of 5 independent experiments performed with 3 different donors; the difference in ATP levels between HK2 transduced T-cells and Control cells is statistically significant (calculated using a Student's paired t-test). FCCP represents a control used as an inhibitor to ATP production.

FIG. 15D. ATP production in PFK engineered T cells. ATP levels in PBLs transduced with PFK or NGFR (control) was measured using ATP reagent Cell titerGlo 2.0 assay ATP levels were normalized to control (NGFR). The results are the mean+SEM of 3 independent experiments with 3 different donors; the difference in ATP levels between PFK transduced cells and NGFR transduced cells is statistically significant (calculated using a Student's paired t-test). FCCP represents a control used as an inhibitor to ATP production.

FIG. 16A-16C. Increased Mitochondrial mass in engineered T-cells

FIG. 16A. Histogram presentation of a FACS analysis of mitochondrial staining in HK2 transduced T cells and NGFR transduced T cells using the mitochondrial probe MitoSpy Green FM. MFI was measured by flow cytometry gated on the HK2 population. The results are the mean+SEM of at least 3 independent experiments with 3 different donors; the difference in mitochondrial mass between HK2 transduced cells and NGFR transduced cells (control) is statistically significant (p<0.05 calculated using a Student's paired t-test)

FIG. 16B. Representative result of FIG. 16A. The MFI of IC (isotype control), NGFR and HK2 was indicated.

FIG. 16C. Histogram presentation of a FACS analysis of mitochondrial staining in PFK or PFK-I-GLUT3 transduced T-cells and NGFR transduced T-cells using the mitochondrial probe MitoSpy Green FM. MFI was measured by flow cytometry gated on the PFK population. The results are the mean+SEM of 3 independent experiments with 3 different donors; the difference in mitochondrial mass between PFK transduced cells or PFK-I-GLUT3 transduced cells and NGFR transduced cells (control) is statistically significant (p<0.05 calculated using a Student's paired t-test).

FIG. 17A-17B. Expression levels of PFK in transduced T-cells over time FIG. 17A. FACS analysis of PFK levels in PBLs untransduced (w/o), transduced with NGFR (control) or transduced with PFK—I-NGFR using an anti-PFK antibody (and goat anti-rabbit FITC as a secondary antibody) on days 9, 14, 23 following transduction. The MFI of positive cells is shown in brackets. The results are representative of 4 different experiments with 3 different donors.

FIG. 17B. FACS analysis of PFK levels in PBLs untransduced (w/o), transduced with NGFR (control) or transduced with PFK-I-GLUT3 using anti-PFK antibody (and goat anti-rabbit FITC as a secondary antibody) on days 9, 14, 23 following transduction. The MFI of positive cells is shown in brackets. The results are representative of 4 different experiments with 3 different donors.

FIG. 18A-18F. in-vivo therapeutic efficacy of engineered T cells

FIG. 18A. graph showing tumor growth of SKMEL23 tumor melanoma cells in the presence of PFK transduced T-cells or control cells in an in vivo model. NSG mice inoculated with melanoma SKMEL23 tumor cells. One week after tumor establishment, NGFR+TCR F5 (control) and PFK+TCR F5 engineered T-cells were injected in mice. Tumor growth was measured in a blinded fashion using a caliper and calculated using the formula: (Dxd²)×Π/6, where D is the largest tumor diameter and d its perpendicular one. The difference in tumor volume between the NGFR and PFK treated groups is statistically significant (p-value=0.0017, calculated using a Student's paired t-test; n=6).

FIG. 18B. graph showing tumor growth of 888A2 tumor melanoma cells in the presence of PFK transduced T-cells or control cells in an in vivo model. NSG mice inoculated with Melanoma 888A2 tumor cells. One week after tumor establishment, NGFR+TCR F5 (control) and PFK+TCR F5 engineered T-cells were injected in mice. Tumor growth was measured in a blinded fashion using a caliper and calculated using the following formula: (Dxd²)×Π/6, where D is the largest tumor diameter and d its perpendicular one. The difference in tumor volume between NGFR and PFK treated groups is statistically significant (p-value=0.049, calculated using a Student's paired t-test; n=5).

FIG. 18C. graph showing tumor growth of SKMEL23 tumor melanoma cells in the presence of GLUT3 transduced T-cells or control cells in an in vivo model. NSG mice inoculated with Melanoma SKMEL23 tumor cells. One week after tumor establishment, NGFR+TCR F5 (control) or GLUT3+TCR F5 engineered T-cells were injected in mice. Tumor growth was measured in a blinded fashion using a caliper and calculated using the following formula: (Dxd²)×Π/6, where D is the largest tumor diameter and d its perpendicular one. The difference in tumor volume between the control and GLUT3 treated groups is statistically significant (p-value=0.016, calculated using a Student's paired t-test; n=5).

FIG. 18D. graph showing tumor growth of 888A2 tumor melanoma cells in the presence of GLUT3 transduced T-cells or control cells in an in vivo model.

NSG mice inoculated with Melanoma 888A2 tumor cells. One week after tumor establishment, NGFR+TCR F5 (control) or GLUT3+TCR F5 engineered T-cells were injected in mice. Tumor growth was measured in a blinded fashion using a caliper and calculated using the following formula: (Dxd²)×Π/6, where D is the largest tumor diameter and d its perpendicular one. The difference in tumor volume between the control and GLUT3 treated groups is statistically significant (p-value=0.037, calculated using a Student's paired t-test; n=5).

FIG. 18E. graph showing tumor growth of SKMEL23 tumor melanoma cells in the presence of PFK-GLUT3 transduced T-cells or control cells in an in vivo model. NSG mice inoculated with Melanoma SKMEL23 tumor cells. One week after tumor establishment, NGFR+TCR F5 (control) and PFK-I-GLUT3+TCR F5 engineered T-cells were injected in mice. Tumor growth was measured in a blinded fashion using a caliper and calculated using the following formula: (Dxd²)×Π/6, where D is the largest tumor diameter and d its perpendicular one. The difference in tumor volume between the control and PFK-I-GLUT3 treated groups is statistically significant (p-value=0.016, calculated using a Student's paired t-test; n=6).

FIG. 18F. graph showing tumor growth of 888A2 tumor melanoma cells in the presence of T-cells expressing a MART-1 specific TCR and HK2 or NGFR (control) in an in vivo model. NSG mice inoculated with Melanoma 888A2 tumor cells. One week after tumor establishment, control cells or HK2+TCR engineered T-cells were injected in mice. Tumor growth was measured in a blinded fashion using a caliper and calculated using the following formula: (Dxd²)×Π/6, where D is the largest tumor diameter and d its perpendicular one. The difference in tumor volume between the control and HK2 treated groups is statistically significant (p-value=0.02, calculated using a Student's paired t-test; n=6).

FIG. 19A-19C. Expression levels of TCR F4, CAR-CD19 41BB and PKM levels in engineered transduced cells

FIG. 19A. FACS analysis of PKM transduced cells in two systems: TCR system in which lymphocytes retrovirally co-transduced with F4 TCR and with either PKM or mock (upper panel) or CAR system in which lymphocytes retrovirally co-transduced with CAR CD19 41BB and either PKM or mock. Transduction efficiency in both systems, F4-TCR and CAR CD19 41BB was measured by flow cytometry 72 h post transduction using Protein L antibody. The percentage of positive cells and the MFI are shown.

FIG. 19B. Histogram presentation of a RT-PCR analysis for PKM expression in F4 TCR transduced cells with either PKM or mock. Expression levels normalized to L13a (housekeeping gene).

FIG. 19C. Western Blot analysis for PKM expression in transduced F4 TCR cells or transduced CAR CD19 41BB cells using Beta actin was used for loading control. The results are representative of 6 independent experiments. The difference in PKM expression levels between the transduced population and the non-transduced population is statistically significant (p<0.05; calculated using a Student's paired t-test).

FIG. 20A-20B. Enhanced cytokine secretion by PKM-induced T cells

FIG. 20A(i)-20A(iii). ELISA analysis for IFNγ (FIG. 20Ai), TNFα (FIG. 20Aii) and IL-2 (FIG. 20Aiii) secretion in culture supernatant of lymphocytes retrovirally co-transduced with F4 TCR and with either PKM or mock and co-cultured with negative target cells (A375) and positive target cells (888A2).

FIG. 20B(i)-20B(iii). ELISA analysis for IFNγ (FIG. 20Bi), TNFα (FIG. 20Bii) and IL-2 (FIG. 20Biii) in culture supernatant of lymphocytes retrovirally co-transduced with CAR CD1941BB and with either PKM or mock and co-cultured with negative target cells (K562) and positive target cells (K562-CD19) The results are representative of 4 independent experiments the difference in the concentration of each of the cytokines between the transduced population and the non-transduced population is statistically significant (p<0.05; calculated using a Student's paired t-test).

FIG. 21A-21E. Elevated levels of activation markers in PKM-induced T cells

FIG. 21A(i)-21A(iii). FACS analysis for the expression of 41BB (also known as CD137) (FIG. 21Ai), CD25 (FIG. 21Aii) or, CD69 (FIG. 21Aiii) markers in transduced cells. PKM or mock-transduced T cells were cultured after OKT3 activation and the 41BB, CD25 marker or, CD69 marker were measured by FACS using anti-CD137, anti-CD25 and anti-CD69 antibodies. The difference between 41BB (CD137) and CD25 expression levels between PKM or mock-transduced T cells is statistically significant (calculated using a Student's paired t-test).

FIG. 21B. FACS analysis for the expression of 41BB (also known as CD137) in transduced cells. PKM or mock-transduced T cells were co-transduced with CAR-CD19 41BB. Upon activation the expression levels of CD137 marker was evaluated by FACS using anti-CD137. The difference in CD137 expression levels between PKM or mock-transduced T cells is statistically significant calculated using a Student's paired t-test as indicated.

FIG. 21C. Cell proliferation assay for Mock and PKM transduced T cells activated with OKT3, labeled with 5 uM CFSE and cultured for 3 days. Proliferation ratio was calculated between day 0 and day 3.

FIG. 21D. Histogram presentation of a Real-time PCR of CCND1 (encoding for Cyclin D1), normalized to L13a.

FIG. 21E. Memory immunophenotype of lymphocytes retrovirally co-transduced with CAR CD19 41BB and either PKM or mock using CD45RO and CCR7 antibodies to characterize percentage of effector and central memory T cells. The results are representative of 3 independent experiments. The difference in the central memory population between the CAR CD19 PKM transduced cells CAR CD19 mock transduced cells is statistically significant (p<0.05; calculated using a Student's paired t-test).

FIG. 22A-22C. PKM drives the TCA cycle and production of ATP

FIG. 22A(i)-22A(iv). LC-MS analysis of the metabolites Cis-aconitate (FIG. 22Ai), alpha-ketoglutarate (aKG) (FIG. 21Aii), Succinic acid (FIG. 21Aiii) and Malate (FIG. 21Aiv) in PKM- and mock-transduced T cells.

[U-13C6]-glucose tracing was performed for 30 min in PKM- and mock-transduced T cells (n=3) followed by LC-MS analysis to evaluate intermediates levels of the TCA cycle.

FIG. 22B. ATP content in PKM- and mock-transduced T cells. Results are shown as normalized mock and to AUG (of ug protein). Basal levels (white bar) and M+2 isotopologues of each intermediate (blue bar) are presented. Bars represent mean±SD. Statistical differences were determined by two-tailed Student's t-test.

FIG. 22C. ATP levels in PBLs transduced with PKM or Mock (control) was measured in regular and in glucose-free media using ATP Cell titerGlo 2.0 assay reagent. ATP levels were normalized to Mock (Control). The results are the mean+SEM of 3 independent experiments with 3 different donors; the difference in ATP levels between PKM transduced cells and NGFR transduced cells is statistically significant (calculated using a Student's paired t-test)

FIG. 23A-23C. PKM-transduced T cells exhibit higher cytotoxicity and antitumoral activity

FIG. 23A. Cell cytotoxicity assay of T-cells co-transduced with F4 TCR and PKM or empty vector (Mock). The transduced cells (Effector (E) cells) were co-culture with positive 888A2 melanoma cells (Target (T) cells) at E:T ratio 2:1. Images were captured hourly over 24 hrs using the Sartorius IncuCyte® system. Mean fluorescence of live cells was calculated. Statistical differences were determined by one-tailed Student's t-test.

FIG. 23B. graph showing tumor growth of 888A2 tumor melanoma cells in the presence of PKM transduced T-cells in NSG an in vivo model.

Mice (n=7/group) were injected with 2 million 888A2 melanoma cells and on day 7, when tumors reached 5 mm, 5×10⁶ F4 T-cells expressing PKM- or mock-transduced T cells were injected I.V. (first I.V injection) into the mice followed by a second I.V injection a week later. Tumors volumes were measured.

FIG. 23C. Survival rate by Kaplan-Meier analysis in mice injected with 888A2 tumor melanoma cells in the presence of F4-PKM transduced T-cells or F4-mock-transduced T cells. Mice (n=5/group) were injected with 2 million 888A2 melanoma cells and on day 7, when tumors reached 5 mm, 5×10⁶ F4 T-cells expressing PKM- or mock-transduced T cells were injected I.V. (first I.V injection) into the mice followed by a second I.V injection a week later. Tumors volumes were measured.

### DETAILED DESCRIPTION OF EMBODIMENTS

In some embodiments, the engineered cells of the present disclosure express at least one protein participating, either directly or indirectly in glycolysis. In each step of the glycolysis, important enzymes catalyze reactions. The main reactions in the glycolysis pathway are catalyzed by hexokinase, phosphofructokinase, and pyruvate kinase; hence, these enzymes have a regulatory role. They are considered as ‘control sites’[Berg J M. et al., (2002) ibid.].

Hexokinase (HK) is an enzyme which catalyzes the first step of the glycolysis pathway. It mediates the irreversible first step of the glycolytic pathway by catalyzing the phosphorylation of D-glucose to D-glucose 6-phosphate with concomitant de-phosphorylation of ATP. Hexokinase binds glucose after his entry into the cell by glucose transporters and directly metabolize it. In mammalian tissues, 4 isoforms of Hexokinase enzyme have been characterized: HK I, II, III and IV (glucokinase). HK I, II and III have a high affinity for the substrate glucose (K_{M}˜0.02 mM) [Mathupala, S. P., et al., Oncogene, 25(34), 4777-4786 (2006)] and are even more specific than glucose transporters to glucose. HK initiates major pathways of intracellular glucose utilization, and HK type II combines glycolysis to oxidative phosphorylation by interacting with mitochondria, thus acting as a metabolic sensor [Chiara, F., et al., PLoS ONE, 3(3) (2008)]. In aggressive tumors, mitochondrial HK II activity is increased in hypoxic conditions.

In addition, Hexokinase plays a key role in maintaining the integrity of the outer mitochondrial membrane by preventing the release of apoptogenic molecules from the intermembrane space and subsequent apoptosis [Chiara, F., et al., ibid.].

Phosphofructokinase (PFK) is an important control element in the glycolytic pathway. PFK1 catalyzes the third step of the glycolysis, the conversion of the fructose-6-phosphate into fructose-1,6-phosphate; this step is ATP-dependent. PFK1 is an allosteric enzyme that can regulate glycolysis through allosteric inhibition or activation, and in this way, the cell can increase or decrease the rate of glycolysis in response to the cell's energy requirements. The enzyme activity depends on the ATP/AMP ratio; when it is lowered, the enzyme is increased. The enzyme is also inhibited by a low pH. PFK1 can also communicate with the Krebs cycle and its pyruvate needs via a TCA substrate called citrate. A high level of citrate means that biosynthetic precursors are abundant and additional glucose should not be degraded for this purpose. Citrate inhibits phosphofructokinase by enhancing the inhibitory effect of ATP [Berg J M. et al., ibid]. Mammalian PFK1 is a tetramer composed differently according to the tissue type it is present in. There are three isoforms in mammalian tissue—muscle (PFKM), liver (PFKL) and platelet (PFKP).

Several studies proved that an over activation of the PFK enzyme in the tumor can promote cell proliferation and tumorigenesis [Lee, J. H., et al., Molecular Cell, 70(2), 197-210.e7 (2018; Bartrons, R., et al., Frontiers in Oncology, 8(SEP) (2018)].

Glucose is a major source of energy in mammalian cells, generating ATP through glycolysis and oxidative phosphorylation. It is also a precursor of amino acids, nucleotides and lipids [Li, B., & Pan, F. (Eds.). ibid.]. The transport of glucose across the plasma membrane into the cytosol is a rate-limiting step in glucose metabolism and is mediated by a family of glucose transporters (GLUTs) [Berg J M. et al., ibid]. Glucose transporters are uni/transporter protein. There are 14 types of transporters in the glucose transporter family. They are integral membrane proteins that contain 12 membrane-spanning helices [Navale, A. M., & Paranjape, A. N. Biophysical Reviews, 8(1), 5-9 (2016)]. GLUTs facilitate the entrance of glucose in the cell. The binding of glucose causes a conformational change associated with transport and releases glucose to the other side of the membrane.

In cancer, the glycolysis pathway is often upregulated and the dependence on glucose is increased [Li, B., & Pan, F. (Eds.). ibid.]. Abnormal cells can also exploit the glucose from other cells, and mainly from immune system cells by overexpressing glucose transporters. Various studies have demonstrated that inhibition of glucose transport results in apoptosis and can also decrease cancer cell proliferation [Zhang, W., Liu, Y., Chen, X., & Bergmeier, S. C. Bioorganic and Medicinal Chemistry Letters, 20(7), 2191-2194 (2010)].

GLUT3 is mostly found in nerve cells, where it is thought to be responsible for most of the glucose transport.

GLUT3 are present in nearly all mammalian cells, have a key role in glucose uptake in the cell. Their K_{M} (Michaelis-Menten constant) value for glucose is low, about 1 mM, significantly less than the normal serum-glucose level, which typically ranges from 4 mM to 8 mM [Berg J M, Biochemistry. 5th edition. New York: W H Freeman; (2002)]. Thus, glucose transporters require only a small amount of substrate to become saturated and then, have a high affinity to glucose. Hence, GLUT3 continually transport glucose into cells at an essentially constant rate.

Pyruvate kinase (PK) plays a pivotal role in regulating cell metabolism, by catalyzing the conversion of Phosphoenolpyruvate (PEP) to Pyruvate [Otto et al., (2016) ibid.]. While there are four isomeric, tissue-specific forms of Pyruvate Kinase found in mammals, PKM2 has gained most attention due to its many roles in glucose metabolism [Zhang Z. et al, Cell Biosci. 9: 52 (2019)]. In the tetrameric form PKM is associated with routing glucose metabolism to pyruvate into the tricarboxylic acid cycle, converting to the pentose phosphate pathway [Lunt et al., Mol Cell. 57(1): 95-107 (2015)), the uronic acid pathway, and the polyol pathway [Luo W. et al., Trends Endocrinol Metab. 23:560-6 (2012); Israelsen W J. et al., Cell. 155:397-409 (2013); Israelsen W J., and Heidena M G V., Semin Cell Dev Biol. (2015); Dong et al., Oncology Letters. 11:1980-6 (2016)]. In the monomeric/dimeric form however, PKM2 can exist in a variety of different intracellular localizations, enter the nuclear to regulate gene expression, and attaches to the mitochondrial outer membrane to maintain mitochondrial function [Christofk H. Nature. 452:181-6 (2008); Israelsen and Heiden, ibid. (2015), Dong et al., ibid. (2016); Dayton et al., EMBO Rep. 17:1721-30 (2016)].

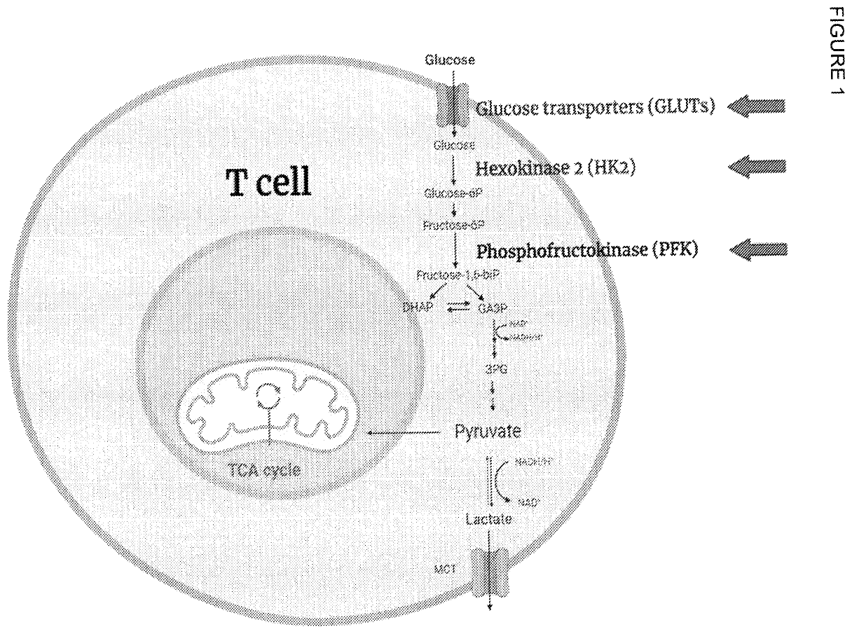

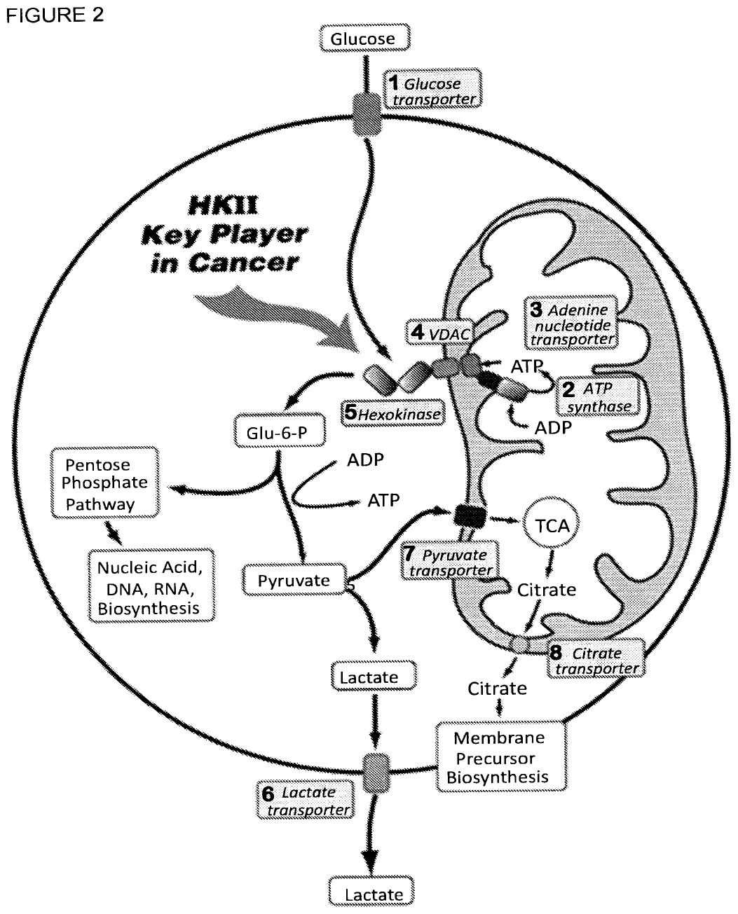

The goal of the present disclosure is improving lymphocyte function by endowing them with some characteristics of the tumor cell, by overexpression of genes involved in metabolic pathways such as those related to glycolysis and amino acid. The inventors focused on the overexpression of glucose transporters or key enzymes in glycolysis pathways (FIG. 1) such as Hexokinase-2 (HK2), Phosphofructokinase (PFK), pyruvate kinase (PKM), Glucose transporters (GLUT3), or their isoforms, and combinations thereof, and showed to substantially enhance lymphocyte function and, thus, contribute to the improvement of engineered T cell-based immunotherapy.

Thus, a first aspect of the present disclosure relates to at least one genetically engineered hematopoietic cells or a cell population comprising at least one of the cell/s. More specifically, the genetically engineered hematopoietic cell/s in accordance with the present disclosure comprises and/or expresses at least one nucleic acid sequence encoding at least one molecule involved directly or indirectly in at least one metabolic pathway. In some embodiments, the nucleic acid sequence may be exogenous nucleic acid sequence. In some embodiments, the genetically engineered hematopoietic cell/s of the present disclosure may optionally further express, or may be engineered to further express, at least one receptor molecule. The present disclosure provides a genetically edited and/or engineered cells. A genetically engineered cell, as used herein, is a cell that has been modified through the manipulation of its genetic material, typically using techniques such as gene editing or gene transfer. This manipulation can involve the addition, deletion, or modification of specific genes or segments of DNA within the genome of the engineered cell. The disclosed genetically engineered cells of the present disclosure may exogenously comprise and/or express the disclosed nucleic acid sequence. “Exogenously express” refers to the introduction or addition of genetic material or a foreign substance into a cell or organism from an external source, typically through artificial means, as disclosed herein, for example, by transfection, transduction using any nucleic acid vectors, sequences and compositions.

Still further, in some embodiments, the engineered hematopoietic cells of the present disclosure may be non-erythroid hematopoietic cell. In yet some further embodiments, the non-erythroid hematopoietic cells in accordance with the present disclosure may be in some embodiments, lymphoid cells. In yet some further embodiments the engineered cell/s of the present disclosure may be any lymphocyte. In yet some further embodiments, the engineered lymphocytes of the present disclosure may be any cell of the T lineage, for example, T cells and/or NK, and/or NKT, and/or MAIT cells. In yet some further embodiments, the engineered lymphocyte in accordance with the present disclosure may be of the B lineage. “Hematopoietic cells” are cellular blood components all derived from hematopoietic stem cells in the bone marrow. It should be appreciated that in certain embodiments, hematopoietic cells as used herein include cells of the myeloid and the lymphoid lineages of blood cells. More specifically, myeloid cells include monocytes, (macrophages and dendritic cells (DCs)), granulocytes (neutrophils), basophils, eosinophils, erythrocytes, and megakaryocytes or platelets. The Lymphoid cells include T cells, B cells, and natural killer (NK) cells. Thus, in certain embodiments, the engineered hematopoietic cells of the present disclosure may be any hematopoietic cell described herein. Generally, blood cells are divided into three lineages: red blood cells (erythroid cells) which are the oxygen carrying, white blood cells (leukocytes, that are further subdivided into granulocytes, monocytes and lymphocytes) and platelets (thrombocytes).

In certain embodiments, the engineered hematopoietic cells of the present disclosure may be non-erythroid hematopoietic cells. The term “non-erythroid hematopoietic cell” refers to the cells derived from white blood cell precursors and from megakaryocytes and include at least one of granulocytes (neutrophils, basophils, eosinophils), monocytes, lymphocytes, macrophages, dendritic cells and platelets.

As indicted in the present aspect, the present disclosure provides an engineered cell that may be any lymphocyte, specifically, any lymphocyte of the T lineage. “Lymphocytes” are mononuclear nonphagocytic leukocytes found in the blood, lymph, and lymphoid tissues. They are divided on the basis of ontogeny and function into two classes, B and T lymphocytes, responsible for humoral and cellular immunity, respectively. Most are small lymphocytes 7-10 μm in diameter with a round or slightly indented heterochromatic nucleus that almost fills the entire cell and a thin rim of basophilic cytoplasm that contains few granules. When “activated” by contact with antigen, small lymphocytes begin macromolecular synthesis, the cytoplasm enlarges until the cells are 10-30 μm in diameter, and the nucleus becomes less completely heterochromatic; they are then referred to as large lymphocytes or lymphoblasts. These cells then proliferate and differentiate into B and T memory cells and into the various effector cell types: B cells into plasma cells and T cells into helper, cytotoxic, and suppressor cells.

Still further, in some embodiments, the engineered cell of the present disclosure is a cell of the T lineage. A “T cell” or “T lymphocyte” as used herein is characterized by the presence of a T-cell receptor (TCR) on the cell surface. It should be noted that T-cells include helper T cells (“Th cells”), cytotoxic T cells (“Tc,” “CTL” or “killer T cell”), memory T cells, and regulatory T cells as well as Natural killer T cells, Mucosal associated invariants and Gamma delta T cells.

More specifically, Thymocytes are hematopoietic progenitor cells present in the thymus. Thymopoiesis is the process in the thymus by which thymocytes differentiate into mature T lymphocytes. The thymus provides an inductive environment, which allows for the development and selection of physiologically useful T cells. The processes of beta-selection, positive selection, and negative selection shape the population of thymocytes into a peripheral pool of T cells that are able to respond to foreign pathogens and are immunologically tolerant towards self-antigens.

Thymocytes are classified into a number of distinct maturational stages based on the expression of cell surface markers. The earliest thymocyte stage is the double negative (DN) stage (negative for both CD4 and CD8), which more recently has been better described as Lineage-negative, and which can be divided into four sub-stages. The next major stage is the double positive (DP) stage (positive for both CD4 and CD8). The final stage in maturation is the single positive (SP) stage (positive for either CD4 or CD8).

More specifically, the maturational stages of thymocytes may include the following substages: Double negative 1 (DN1) or ETP (Early T lineage Progenitor) is characterized by CD44+CD25-CD117+ defining surface markers, thymocytes are located in the cortex and proliferation, loss of B and myeloid potentials are observed; Double negative 2 (DN2) is characterized by CD44+CD25+CD117+ defining surface markers and thymocytes are located in the cortex; Double negative 3 (DN3) is characterized by CD44−CD25+ defining surface markers, thymocytes are located in the cortex and TCR-beta rearrangement and beta selection are observed; Double negative 4 (DN4) is characterized by CD44−CD25-defining surface markers and thymocytes are located in the cortex; Double positive is characterized by CD4+CD8+ defining surface markers, thymocytes are located in the cortex and TCR-alpha rearrangement, positive selection, negative selection are observed; Single positive is characterized by CD4+CD8− or CD4−CD8+ defining surface markers, thymocytes are located in the medulla and Negative selection is observed.

In human, circulating CD34+ hematopoietic stem cells (HSC) reside in bone marrow. They produce precursors of T lymphocytes, which seed the thymus (thus becoming thymocytes) and differentiate under influence of the Notch and its ligands. Early, double negative thymocytes express (and can be identified by) CD2, CD5 and CD7. Still during the double negative stage, CD34 expression stops and CD1 is expressed. Expression of both CD4 and CD8 makes them double positive and matures into either CD4+ or CD8+ cells. It should be appreciated that a cell of the T lineage as disclosed herein may be any of the thymocytes disclosed herein at any stage/substage and/or expressing any of the disclosed markers.

Still further, in some embodiments of the present disclosure, the engineered cell of the T lineage, is a T cell. For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4.sup.+/CD8.sup.+double positive T cells, CD4.sup.+helper T cells, e.g., Th.sub.1 and Th.sub.2 cells, CD8.sup.+ T cells (e.g., cytotoxic T cells), tumor infiltrating cells, memory T cells, naive T cells, and the like. The T cell may be a CD8.sup.+ T cell or a CD4.sup.+ T cell.

In some embodiments of the present disclosure, the engineered cell of the T lineage may be a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes. NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human).

Also provided by some embodiments of the present disclosure is a population of cells comprising at least one engineered cell of the T lineage, specifically, the genetically engineered cell of the T lineage described herein. The population of cells can be a heterogeneous population comprising the host cell comprising any of the nucleic acid sequences, cassettes and vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells, specifically, the genetically engineered cell (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the present disclosure, the population of cells is a clonal population comprising host cells that are genetically edited and/or comprising the nucleic acid sequences, cassettes and vectors as described herein.

The present disclosure provides cells, specifically, hematopoietic cells, specifically, lymphocytes, and more specifically, cells of the T lineage that were genetically engineered to express the at least one molecule involved directly or indirectly in at least one metabolic pathway and optionally, the at least one receptor molecules disclosed herein. It should be however noted that the present disclosure further encompasses any host cell comprising, transfected by, transformed and/or engineered and/or edited by nucleic acid sequence, cassette or vector encoding at least one molecule involved directly or indirectly in at least one metabolic pathway and optionally, the at least one receptor as disclosed herein.

The term “host cell” includes a cell into which a heterologous (e.g., exogenous) nucleic acid or protein has been introduced. Persons of skill upon reading this disclosure will understand that such terms refer not only to the particular subject cell, but also is used to refer to the progeny of such a cell. Because certain modifications may occur in succeeding generations due to either mutation or environmental influences, such progeny may not, in fact, be identical to the parent cell, but are still included within the scope of the term “host cell”. As used herein, a cell has been “transformed” or “transfected” by exogenous or heterologous DNA, e.g., the nucleic acid molecule/s of the disclosure or any cassette of the disclosure, when such DNA has been introduced inside the cell. The transforming DNA may be integrated (covalently linked) into the genome of the cell. With respect to the present disclosure, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. It should be appreciated that in some embodiments, the host cells of the disclosure may be any engineered T cells of the disclosure or any cell population comprising, at least in part, the T cells of the disclosure. Still further, the present disclosure further encompasses any population of cells comprising at least 5%, 10%, 15%, 20%, 25%, 30%, 35%, 40%, 45%, 50%, 55%, 60%, 65%, 70%, 75%, 80%, 85%, 90%, 95%, 99.9% or more, specifically, 100%) specifically, 10%, 15%, 20%, 25%, 30%, 35%, 40%, 45%, 50%, 55%, 60%, 65%, 70%, 75%, 80%, 85%, 90%, 95%, 99.9% or more, specifically, 100% of the host cells of the disclosure.

The engineered cells disclosed herein, express and/or is engineered to express at least one molecule involved directly and/or indirectly in at least one metabolic pathway. The term metabolic pathway, as used herein, refers to a linked series of chemical reactions occurring within a cell. The reactants, products, and intermediates of an enzymatic reaction are known as metabolites, which are modified by a sequence of chemical reactions catalyzed by enzymes. In most cases of a metabolic pathway, the product of one enzyme acts as the substrate for the next. However, side products are considered waste and removed from the cell. Metabolism comprises all chemical reactions that occur within living organisms. The processes that involve degradation of molecules correspond to catabolism and those leading to synthesis of new molecules correspond to anabolism. In general, catabolism has an oxidative nature and uses coenzymes like NAD⁺ as an electron acceptor. It is exergonic; the −ΔG is used to transfer phosphate to ADP for ATP synthesis. In contrast, anabolism involves an endergonic process that uses ATP as the main source of energy. In general, it involves reducing reactions and uses NADPH as the H⁺ ion supplier. In the normal adult, both catabolism and anabolism are in equilibrium.

In some embodiments, the molecule involved directly or indirectly in at least one metabolic pathway expressed by the genetically engineered cell of the present disclosure, may be at least one of: at least one catabolic protein, for example, a protein involves in break down complex molecules and release energy in the process, an anabolic protein, for example, any protein that synthesizes molecules with the utilization of energy, or an amphibolic protein, specifically, a protein that can be either catabolic or anabolic based on the need for or the availability of energy.

The term Catabolic pathway, as used herein, refers to a series of reactions that bring about a net release of energy in the form of a high energy phosphate bond formed with the energy carriers adenosine diphosphate (ADP) and guanosine diphosphate (GDP) to produce adenosine triphosphate (ATP) and guanosine triphosphate (GTP), respectively. The net reaction is, therefore, thermodynamically favorable, for it results in a lower free energy for the final products. A catabolic pathway is an exergonic system that produces chemical energy in the form of ATP, GTP, NADH, NADPH, FADH2, etc. from energy containing sources such as carbohydrates, fats, and proteins. The end products are often carbon dioxide, water, and ammonia. Coupled with an endergonic reaction of anabolism, the cell can synthesize new macromolecules using the original precursors of the anabolic pathway. An example of a coupled reaction is the phosphorylation of fructose-6-phosphate to form the intermediate fructose-1,6-bisphosphate by the enzyme phosphofructokinase accompanied by the hydrolysis of ATP in the pathway of glycolysis. The resulting chemical reaction within the metabolic pathway is highly thermodynamically favorable and, as a result, irreversible in the cell.

The term Catabolism is the set of metabolic pathways that breaks down molecules into smaller units that are either oxidized to release energy or used in other anabolic reactions. Catabolism breaks down large molecules (such as polysaccharides, lipids, nucleic acids, and proteins) into smaller units (such as monosaccharides, fatty acids, nucleotides, and amino acids, respectively). Catabolism is the breaking-down aspect of metabolism, whereas anabolism is the building-up aspect. Cells use the monomers released from breaking down polymers to either construct new polymer molecules or degrade the monomers further to simple waste products, releasing energy. Cellular wastes include lactic acid, acetic acid, carbon dioxide, ammonia, and urea. The formation of these wastes is usually an oxidation process involving a release of chemical free energy, some of which is lost as heat, but the rest of which is used to drive the synthesis of adenosine triphosphate (ATP). This molecule acts as a way for the cell to transfer the energy released by catabolism to the energy-requiring reactions that make up anabolism.

Catabolism is a destructive metabolism and anabolism is a constructive metabolism. Catabolism, therefore, provides the chemical energy necessary for the maintenance and growth of cells. Examples of catabolic processes include glycolysis, the citric acid cycle, the breakdown of muscle protein in order to use amino acids as substrates for gluconeogenesis, the breakdown of fat in adipose tissue to fatty acids, and oxidative deamination of neurotransmitters by monoamine oxidase. Accordingly, in the context of the present disclosure, a catabolic protein as used herein, refers to any protein that is involved in a catabolic pathway as defined above.

The term Anabolic pathway as used herein, refers to the set of metabolic pathways that construct molecules from smaller units. These reactions require energy, known also as an endergonic process. Anabolism is the building-up aspect of metabolism, whereas catabolism is the breaking-down aspect. Anabolism is usually synonymous with biosynthesis. Many anabolic processes are powered by the cleavage of adenosine triphosphate (ATP). Anabolism usually involves reduction and decreases entropy, making it unfavourable without energy input. The starting materials, called the precursor molecules, are joined using the chemical energy made available from hydrolyzing ATP, reducing the cofactors NAD+, NADP+, and FAD, or performing other favourable side reactions. Occasionally it can also be driven by entropy without energy input, in cases like the formation of the phospholipid bilayer of a cell, where hydrophobic interactions aggregate the molecules. Accordingly, in the context of the present disclosure, an anabolic protein as used herein, refers to any protein that is involved in the anabolic pathway as defined above.

The term amphibolic as used describe a biochemical pathway that involves both catabolism and anabolism as defined above. This term emphasizes the dual metabolic role of such pathways. All the reactions associated with synthesis of biomolecule converge into the following pathway, viz., glycolysis, the Krebs cycle and the electron transport chain, exist as an amphibolic pathway, meaning that they can function anabolically as well as catabolically. Other exemplary important amphibolic pathways are the Embden-Meyerhof pathway, the pentose phosphate pathway and the Entner-Doudoroff pathway. Accordingly, in the context of the present disclosure, an amphibolic protein as used herein, refers to any protein that is involved in the amphibolic pathway as defined above.

In some embodiments, the molecule involved directly or indirectly in at least one metabolic pathway expressed by the genetically engineered cell of the present disclosure, may be a protein molecule. Specifically, a protein molecule involved with any metabolic pathway, specially, at least one of: glycolysis pathway, pentose phosphate pathway, fatty acid biosynthesis pathway, electron transport chain, and/or oxidative phosphorylation.

The term Glycolysis as used herein, refers to a metabolic pathway that converts glucose (C₆H₁₂O₆) into pyruvate via intermediate metabolites. The free energy released in this process is used to form the high-energy molecules adenosine triphosphate (ATP) and reduced nicotinamide adenine dinucleotide (NADH). Glycolysis is a sequence of ten reactions catalysed by enzymes.

Glycolysis is a metabolic pathway that does not require oxygen (in anaerobic conditions pyruvate is converted to lactic acid). The wide occurrence of glycolysis in other species indicates that it is an ancient metabolic pathway. In most organisms, glycolysis occurs in the cytosol. The most common type of glycolysis is the Embden-Meyerhof-Parnas (EMP) pathway. Glycolysis also refers to other pathways, such as the Entner-Doudoroff pathway and various heterofermentative and homofermentative pathways. The glycolysis pathway can be separated into two phases: the first, Investment phase—wherein ATP is consumed and the second, Yield phase—wherein more ATP is produced than originally consumed. The first five steps of Glycolysis are regarded as the preparatory (or investment) phase, since they consume energy to convert the glucose into two three-carbon sugar phosphates (G3P). The first step is phosphorylation of glucose by a family of enzymes called hexokinases to form glucose 6-phosphate (G6P). This reaction consumes ATP, but it acts to keep the glucose concentration low, promoting continuous transport of glucose into the cell through the plasma membrane transporters. In addition, it blocks the glucose from leaking out—the cell lacks transporters for G6P, and free diffusion out of the cell is prevented due to the charged nature of G6P. Glucose may alternatively be formed from the phosphorolysis or hydrolysis of intracellular starch or glycogen. G6P is then rearranged into fructose 6-phosphate (F6P) by glucose phosphate isomerase. Fructose can also enter the glycolytic pathway by phosphorylation at this point. The change in structure is an isomerization, in which the G6P has been converted to F6P. The reaction requires an enzyme, phosphoglucose isomerase, to proceed. Isomerization to a keto sugar is necessary for carbanion stabilization in the fourth reaction step. The energy expenditure of another ATP in this step is justified in 2 ways: The glycolytic process (up to this step) becomes irreversible, and the energy supplied destabilizes the molecule. Because the reaction catalyzed by phosphofructokinase 1 (PFK-1) is coupled to the hydrolysis of ATP (an energetically favorable step) it is, in essence, irreversible, and a different pathway must be used to do the reverse conversion during gluconeogenesis. This makes the reaction a key regulatory point. Furthermore, the second phosphorylation event is necessary to allow the formation of two charged groups (rather than only one) in the subsequent step of glycolysis, ensuring the prevention of free diffusion of substrates out of the cell. Destabilizing the molecule in the previous reaction allows the hexose ring to be split by aldolase into two triose sugars: dihydroxyacetone phosphate (a ketose), and glyceraldehyde 3-phosphate (an aldose). Electrons delocalized in the carbon-carbon bond cleavage associate with the alcohol group. The resulting carbanion is stabilized by the structure of the carbanion itself via Triosephosphate isomerase rapidly interconverts dihydroxyacetone phosphate with glyceraldehyde 3-phosphate (GADP) that proceeds further into glycolysis. This is advantageous, as it directs dihydroxyacetone phosphate down the same pathway as glyceraldehyde 3-phosphate, simplifying regulation. The second half of glycolysis is known as the pay-off phase, characterised by a net gain of the energy-rich molecules ATP and NADH. Since glucose leads to two triose sugars in the preparatory phase, each reaction in the pay-off phase occurs twice per glucose molecule. This yields 2 NADH molecules and 4 ATP molecules, leading to a net gain of 2 NADH molecules and 2 ATP molecules from the glycolytic pathway per glucose.

The aldehyde groups of the triose sugars are oxidised, and inorganic phosphate is added to them, forming 1,3-bisphosphoglycerate. The hydrogen is used to reduce two molecules of NAD⁺, a hydrogen carrier, to give NADH+H⁺ for each triose. Hydrogen atom balance and charge balance are both maintained because the phosphate (Pᵢ) group actually exists in the form of a hydrogen phosphate anion (HPO2-4), which dissociates to contribute the extra H⁺ ion and gives a net charge of −3 on both sides. Arsenate ([AsO₄]³⁻), an anion akin to inorganic phosphate may replace phosphate as a substrate to form 1-arseno-3-phosphoglycerate. This, however, is unstable and readily hydrolyzes to form 3-phosphoglycerate, the intermediate in the next step of the pathway. As a consequence of bypassing this step, the molecule of ATP generated from 1-3 bisphosphoglycerate in the next reaction will not be made, even though the This step is the enzymatic transfer of a phosphate group from 1,3-bisphosphoglycerate to ADP by phosphoglycerate kinase, forming ATP and 3-phosphoglycerate. At this step, glycolysis has reached the break-even point: 2 molecules of ATP were consumed, and 2 new molecules have now been synthesized. This step, one of the two substrate-level phosphorylation steps, requires ADP; thus, when the cell has plenty of ATP (and little ADP), this reaction does not occur. Because ATP decays relatively quickly when it is not metabolized, this is an important regulatory point in the glycolytic pathway.

Phosphoglycerate mutase isomerises 3-phosphoglycerate into 2-phosphoglycerate.

Enolase next converts 2-phosphoglycerate to phosphoenolpyruvate. This reaction is an elimination reaction involving an E1cB mechanism.

A final substrate-level phosphorylation now forms a molecule of pyruvate and a molecule of ATP by means of the enzyme pyruvate kinase. This serves as an additional regulatory step, similar to the phosphoglycerate kinase step.

In some embodiments, the metabolic pathway may be the pentose phosphate pathway. The term Pentose phosphate pathway as used herein, refers to a metabolic pathway which is parallel to glycolysis. It generates NADPH and pentoses (5-carbon sugars) as well as ribose 5-phosphate, a precursor for the synthesis of nucleotides. While the pentose phosphate pathway does involve oxidation of glucose, its primary role is anabolic rather than catabolic. There are two distinct phases in the pathway. The first is the oxidative phase, in which NADPH is generated, and the second is the non-oxidative synthesis of 5-carbon sugars. The primary results of the pathway are: a) the generation of reducing equivalents, in the form of NADPH, used in reductive biosynthesis reactions within cells (e.g. fatty acid synthesis). b) production of ribose 5-phosphate (R5P), used in the synthesis of nucleotides and nucleic acids. c) Production of erythrose 4-phosphate (E4P) used in the synthesis of aromatic amino acids.

Some of the enzymes involved in the pentose phosphate pathway include, but are not limited to glucose 6-phosphate dehydrogenase, 6-phosphogluconolactonase, 6-phosphogluconate dehydrogenase, Ribose-5-phosphate isomerase, Ribulose 5-Phosphate 3-Epimerase, transketolase, transaldolase, transketolase, deacetylase SIRT2, etc.

In some embodiments, the metabolic pathway may be the Fatty acid biosynthesis pathway. The term Fatty acid biosynthesis pathway as used herein, refers to the creation of fatty acids from acetyl-CoA and NADPH through the action of enzymes called fatty acid synthases. This process takes place in the cytoplasm of the cell. Most of the acetyl-CoA which is converted into fatty acids is derived from carbohydrates via the glycolytic pathway. The glycolytic pathway also provides the glycerol with which three fatty acids can combine (by means of ester bonds) to form triglycerides (also known as “triacylglycerols”- to distinguish them from fatty “acids”- or simply as “fat”), the final product of the lipogenic process. When only two fatty acids combine with glycerol and the third alcohol group is phosphorylated with a group such as phosphatidylcholine, a phospholipid is formed. Phospholipids form the bulk of the lipid bilayers that make up cell membranes and surrounds the organelles within the cells (such as the cell nucleus, mitochondria, endoplasmic reticulum, Golgi apparatus, etc.). Some of the enzymes involved in the fatty acid biosynthesis pathway, include, but are not limited to atty acid synthase I (FASI), Acetyl CoA:ACP transacylase, Malonyl CoA:ACP transacylase, 3-ketoacyl-ACP synthase, 3-ketoacyl-ACP reductase, 3-Hydroxyacyl ACP dehydrase, Enoyl-ACP reductase, ATP citrate lyase, acetyl CoA carboxylase, etc.

In some embodiments, the metabolic pathway may involve the Electron transport chain (ETC). The term Electron transport chain (ETC) as used herein, refers to a series of protein complexes and other molecules that transfer electrons from electron donors to electron acceptors via redox reactions (both reduction and oxidation occurring simultaneously) and couples this electron transfer with the transfer of protons (H⁺ ions) across a membrane. The electrons that transferred from NADH and FADH2 to the ETC involves 4 multi-subunit large enzymes complexes and 2 mobile electron carriers. Many of the enzymes in the electron transport chain are membrane-bound.

The flow of electrons through the electron transport chain is an exergonic process. The energy from the redox reactions creates an electrochemical proton gradient that drives the synthesis of adenosine triphosphate (ATP). In aerobic respiration, the flow of electrons terminates with molecular oxygen as the final electron acceptor. In anaerobic respiration, other electron acceptors are used, such as sulfate.

In an electron transport chain, the redox reactions are driven by the difference in the Gibbs free energy of reactants and products. The free energy released when a higher-energy electron donor and acceptor convert to lower-energy products, while electrons are transferred from a lower to a higher redox potential, is used by the complexes in the electron transport chain to create an electrochemical gradient of ions. It is this electrochemical gradient that drives the synthesis of ATP via coupling with oxidative phosphorylation with ATP synthase.

In eukaryotic organisms the electron transport chain, and site of oxidative phosphorylation, is found on the inner mitochondrial membrane. The energy released by reactions of oxygen and reduced compounds such as cytochrome c and (indirectly) NADH and FADH2 is used by the electron transport chain to pump protons into the intermembrane space, generating the electrochemical gradient over the inner mitochondrial membrane. According to the chemiosmotic coupling hypothesis, the electron transport chain and oxidative phosphorylation are coupled by a proton gradient across the inner mitochondrial membrane.

In some embodiments, the metabolic pathway may involve Oxidative phosphorylation. The term Oxidative phosphorylation (OXPHOS or electron transport-linked phosphorylation or terminal oxidation) as used herein, refers to the metabolic pathway in which cells use enzymes to oxidize nutrients, thereby releasing chemical energy in order to produce adenosine triphosphate (ATP). In eukaryotes, this takes place inside mitochondria. Almost all aerobic organisms carry out oxidative phosphorylation. This pathway is so pervasive because it releases more energy than alternative fermentation processes such as anaerobic glycolysis.

The energy stored in the chemical bonds of glucose is released by the cell in the citric acid cycle producing carbon dioxide, and the energetic electron donors NADH and FADH. Oxidative phosphorylation uses these molecules and O₂ to produce ATP, which is used throughout the cell whenever energy is needed. During oxidative phosphorylation, electrons are transferred from the electron donors to a series of electron acceptors in a series of redox reactions ending in oxygen, whose reaction releases half of the total energy.

In eukaryotes, these redox reactions are catalysed by a series of protein complexes within the inner membrane of the cell's mitochondria. As indicated above, these linked sets of proteins are called the electron transport chain. In eukaryotes, five main protein complexes are involved, whereas in prokaryotes many different enzymes are present, using a variety of electron donors and acceptors.

The energy transferred by electrons flowing through this electron transport chain is used to transport protons across the inner mitochondrial membrane, in a process called electron transport. This generates potential energy in the form of a pH gradient and an electrical potential across this membrane. This store of energy is tapped when protons flow back across the membrane and down the potential energy gradient, through a large enzyme called ATP synthase in a process called chemiosmosis. The ATP synthase uses the energy to transform adenosine diphosphate (ADP) into adenosine triphosphate, in a phosphorylation reaction. The reaction is driven by the proton flow, which forces the rotation of a part of the enzyme. The ATP synthase is a rotary mechanical motor.

Although oxidative phosphorylation is a vital part of metabolism, it produces reactive oxygen species such as superoxide and hydrogen peroxide, which lead to propagation of free radicals, damaging cells and contributing to disease and, possibly, aging and senescence. Some of the enzymes involved in the oxidative phosphorylation include, but are not limited to NADH-coenzyme Q oxidoreductase (complex I), Succinate-Q oxidoreductase (complex II), Electron transfer flavoprotein-Q oxidoreductase, Q-cytochrome c oxidoreductase (complex III), Cytochrome c oxidase (complex IV), etc.

In yet some further embodiments, the protein involved directly or indirectly in at least one metabolic pathway expressed by the genetically engineered cell of the present disclosure, may be at least one of an enzymatic protein, a transporter protein, a structural protein, an adaptor protein and/or a protein participating in signal transduction related to said at least one metabolic pathway.

An enzymatic protein, as used herein, refers to proteins that act as biological catalysts by accelerating chemical reactions and regulate biochemical processes The molecules upon which enzymes may act are called substrates, and the enzyme converts the substrates into different molecules known as products. Almost all metabolic processes in the cell need enzyme catalysis in order to occur at rates fast enough to sustain life. Metabolic pathways depend upon enzymes to catalyze individual steps. The top-level classification of enzymes include: 1. Oxidoreductases which catalyze oxidation/reduction reactions; 2. Transferases which transfer a functional group (e.g. a methyl or phosphate group); 3. Hydrolases which catalyze the hydrolysis of various bonds; 4. Lyases which cleave various bonds by means other than hydrolysis and oxidation 5. Isomerases which catalyze isomerization changes within a single molecule. 6. Ligases which join two molecules with covalent bonds. 7. Translocases which catalyze the movement of ions or molecules across membranes, or their separation within membranes. These classes are subdivided by other features such as the substrate, products, and chemical mechanism. In the context of the subject disclosure, an enzymatic protein may be one that is involved in any one of the metabolic pathways disclosed therein. In one example, the enzymatic protein may be one that plays a role in the glycolysis pathway.

A structural protein as used herein, refers to any protein that plays a role in shaping the skeletons and structures of cells, tissues, and organisms. Structural proteins are the most abundant class of proteins in nature and their amino acid sequences often show characteristic features, such as a repeating tandem motif. In the context of the subject disclosure, a structural protein may be involved in one or more of the metabolic pathways disclosed therein.

A protein participating in signal transduction as used herein, refers to any protein that participate in the signal transduction. When referring to signal transduction it is understood as any process by which a chemical or physical signal is transmitted through a cell as a series of molecular events, which ultimately results in a cellular response. Proteins responsible for detecting stimuli are generally termed receptors, although in some cases the term sensor is used. The changes elicited by ligand binding (or signal sensing) in a receptor give rise to a biochemical cascade, which is a chain of biochemical events known as a signaling pathway. When signaling pathways interact with one another they form networks, which allow cellular responses to be coordinated, often by combinatorial signaling events. At the molecular level, such responses include changes in the transcription or translation of genes, and post-translational and conformational changes in proteins, as well as changes in their location. These molecular events are the basic mechanisms controlling metabolism, cell growth, proliferation, and many other processes. In multicellular organisms, signal transduction pathways regulate cell communication in a wide variety of ways.

In some embodiments, the protein involved directly or indirectly in said at least one metabolic pathway expressed by the genetically engineered cell of the present disclosure, may be at least one of: Hexokinase (HK), Glucose Transporter Type 3 (GLUT3), Phosphofructokinase-1 (PFK-1), Pyruvate kinase (PKM), Glucose-6-phosphate Isomerase (PGI), Fructose-bisphosphate aldolase (ALDO), Triosephosphate isomerase (TPI), Glyceraldehyde-3-phosphate dehydrogenase (GAPDH), Phosphoglycerate kinase (PGK), Phosphoglycerate mutase (PG) and/or Phosphopyruvate hydratase (enolase).

In some embodiments, Hexokinase (HK) as used herein, refers to any isoform of the human HK. In yet some further embodiments, the human HK is denoted by the UNIPROT accession number P52789. Still further, the human HK as disclosed herein is encoded by the nucleic acid sequence as denoted by genbank accession number BC021116.1. Still further, in some embodiments, the HK as used herein comprises the amino acid sequence as denoted by SEQ ID NO: 1, and any isoform or variant thereof. Still further, in some embodiments, the HK is encoded b a nucleic acid sequence comprising the sequence as denoted by SEQ ID NO: 2, and any homolog, ortholog or variant thereof.

In some embodiments, Phosphofructokinase-1 (PFK-1), as used herein, refers to any isoform of the human PFK-1. In yet some further embodiments, the human PFK is denoted by the UNIPROT accession number P08237. Still further, the human PFK as disclosed herein is encoded by the nucleic acid sequence as denoted by genbank accession number BCO21203. Still further, in some embodiments, the PFK as used herein comprises the amino acid sequence as denoted by SEQ ID NO: 3, and any isoform or variant thereof. Still further, in some embodiments, the PFK is encoded b a nucleic acid sequence comprising the sequence as denoted by SEQ ID NO: 4, and any homolog, ortholog or variant thereof.

In some embodiments, Glucose Transporter Type 3 (GLUT3) as used herein, refers to any isoform of the human GLUT3. In yet some further embodiments, the human GLUT3 is denoted by the UNIPROT accession number P11169. Still further, the human GLUT3 as disclosed herein is encoded by the nucleic acid sequence as denoted by genbank accession number NM_006931.2. Still further, in some embodiments, the GLUT3 as used herein comprises the amino acid sequence as denoted by SEQ ID NO: 5, and any isoform or variant thereof. Still further, in some embodiments, the GLUT3 is encoded b a nucleic acid sequence comprising the sequence as denoted by SEQ ID NO: 6, and any homolog, ortholog or variant thereof.

In some embodiments, Pyruvate kinase (PKM) as used herein, refers to any isoform of the human HK. In yet some further embodiments, the human PKM is denoted by the UNIPROT accession number AAA36449.1. Still further, the human PKM as disclosed herein is encoded by the nucleic acid sequence as denoted by genbank accession number M23725.1. Still further, in some embodiments, the PKM as used herein comprises the amino acid sequence as denoted by SEQ ID NO: 7, and any isoform or variant thereof. Still further, in some embodiments, the PKM is encoded b a nucleic acid sequence comprising the sequence as denoted by SEQ ID NO: 8, and any homolog, ortholog or variant thereof.

Still further, in some embodiments, Glucose-6-phosphate Isomerase (PGI) is the human PGI, as denoted by Uniprot P06744, as also denoted by the amino acid sequence of SEQ ID NO:29, and any isoform or variant thereof. In yet some in some embodiments, Fructose-bisphosphate aldolase (ALDO), is any isoform of the human ALDO, as denoted by Uniprot P04075, as also Denoted by the amino acid sequence of SEQ ID NO:30, and any isoform or variant thereof. In yet some in some embodiments, Triosephosphate isomerase (TPI), is any isoform of the human TPI, as denoted by Uniprot P60174, as also denoted by the amino acid sequence of SEQ ID NO: 31, and any isoform or variant thereof. In yet some in some embodiments, Glyceraldehyde-3-phosphate dehydrogenase (GAPDH), is any isoform of the human GAPDH, as denoted by Uniprot P04406, as also denoted by the amino acid sequence of SEQ ID NO: 32, and any isoform or variant thereof. In yet some in some embodiments, Phosphoglycerate kinase (PGK) is any isoform of the human PGK-1, as denoted by UniprotP00558, as also denoted by the amino acid sequence of SEQ ID NO: 33, and any isoform or variant thereof. In yet some in some embodiments, Phosphoglycerate kinase (PGK), is any isoform of the human PGK-2, as denoted by Uniprot P07205, as also denoted by the amino acid sequence of SEQ ID NO: 34, and any isoform or variant thereof. In yet some in some embodiments, Phosphoglycerate mutase (PG) is any isoform of the human PG, as denoted by Uniprot P18669, as also denoted by the amino acid sequence of SEQ ID NO: 35, and any isoform or variant thereof. In yet some in some embodiments, Phosphoglycerate mutase (PG-1) is any isoform of the human phosphoglycerate mutase 1 (muscle), as denoted by Uniprot P15259, as also denoted by the amino acid sequence of SEQ ID NO: 36, and any isoform or variant thereof. In yet some in some embodiments, Phosphopyruvate hydratase (enolase) is any isoform of the human phosphopyruvate hydratase, as denoted by Uniprot Q6FHV6, as also denoted by the amino acid sequence of SEQ ID NO: 37, and any isoform or variant thereof.

In yet some in some embodiments, Pyruvate Kinase is any isoform of the human Pyruvate Kinase, as denoted by Uniprot P14618-2, as also denoted by the amino acid sequence of SEQ ID NO: NO: 38, and any isoform or variant thereof.

Still further, in some embodiments, the genetically engineered cell/s of the present disclosure express at least one protein involved directly or indirectly in at least one metabolic pathway. In more specific embodiments, such proteins may be at least one of: HK2, PFK-1, GLUT3, PK-M, and/or any combination thereof.

In some embodiments, the genetically engineered cell/s of the present disclosure may further express, either exogenously or endogenously, at least one receptor molecule. In more specific embodiments, such at least one receptor molecule may comprise at least one target binding domain specific against at least one target antigen.

In some embodiments, the genetically engineered cell/s of the present disclosure may further express at least one receptor molecule. In some embodiments, such receptor molecule may be at least one of: (a) at least one T-cell receptor (TCR) molecule specific for at least one target antigen; and/or (b) at least one chimeric antigen receptor (CAR) molecule specific for at least one target antigen.

In some embodiments of the disclosed genetically engineered cells, the cells may further express at least one receptor molecule. The receptor molecule may be either endogenous molecule or alternatively, exogeneous molecule exogenously added to the cell, for example, by genetically editing the cell. In some embodiments, the receptor molecule may be at least one CAR molecule. The term “chimeric protein” relates to proteins created through the joining/fusing of two or more genes that originally coded for separate proteins. Translation of this chimeric/fusion gene results in a single or multiple polypeptides with functional properties derived from each of the original proteins.

Recombinant chimeric/fusion proteins are created artificially by recombinant DNA technology. Chimeric or chimera usually designate hybrid proteins made of polypeptides having different functions, sources or physico-chemical patterns.

Chimeric Antigen Receptor (CAR), as used herein, refers to a recombinant polypeptide comprising at least an extracellular antigen binding domain, a transmembrane domain and an intracellular cytoplasmic signaling domain comprising a functional stimulatory domain. The receptors are chimeric because they couple between extracellular antigen-binding capabilities and intracellular T-cell, activating functions, in a single receptor molecule. CARs have been engineered to give the T cells they are expressed in the new ability to recognize a specific antigen of interest, thereby facilitating an immune reaction against it. For example, the technology is used in immunotherapy for specifically recognizing specific cancer cells' antigens of interest in order to more effectively direct the immune cells towards those target cells and destroy them.

CAR, as used herein, relates to artificial T cell receptors (also known as chimeric T cell receptors, chimeric immuno-receptors). These are engineered receptors, which graft an arbitrary specificity onto an immune effector cell. Typically, these receptors are used to graft the specificity of a monoclonal antibody onto a T cell.

The initial design (also referred to a first generation) joined an antibody-derived scFv to the CD3ζ intracellular signaling domain of the T-cell receptor through hinge and transmembrane domains.

Second generation CARs add intracellular signaling domains from various costimulatory protein receptors (e.g., CD28, 41BB, ICOS) to the cytoplasmic tail of the CAR to provide additional signals to the T cell. More recent, third generation CARs combine multiple signaling domains, such as CD27, CD28, 4-1BB, ICOS, or OX40, to augment potency.

In some embodiments, the genetically engineered cell/s of the present disclosure further expresses at least one receptor molecule, specifically, at least one CAR molecule. In some embodiments such CAR molecule may comprise: (i) at least one target-binding domain that specifically recognizes and binds at least one target antigen; (ii) at least one hinge and at least one transmembrane domain; and (iii) at least one intracellular T cell signal transduction domain.

In some embodiments, the at least one target binding domain of the CAR molecule that is further expressed by the genetically engineered cell/s of the present disclosure, may comprise any affinity molecule, for example, antibody derived molecules, receptors, and/or aptamer/s that specifically recognize and bind the target antigen. In some embodiments, the CAR comprises at least one antibody or any antigen-binding fragment/s, portion/s or chimera/s thereof, specific for a target antigen.

The CAR molecule provided herein, comprises at least one target-binding domain, that may be in some embodiments, any target-recognition element, for example, at least one antibody or any antigen-binding fragments or domains thereof. In yet some further embodiments, the target-recognition element of the CAR molecule of the present disclosure comprises at least one antibody or any antigen-binding fragment/s, portion/s or chimera/s thereof.

Exemplary categories of antigen-binding domains that can be used in the context of the present disclosure include antibodies, antigen-binding portions of antibodies (e.g., single chain variable fragments (scFv)), peptides that specifically interact with a particular antigen (e.g., peptibodies), receptor molecules that specifically interact with a particular antigen, proteins comprising a ligand-binding portion of a receptor that specifically binds a particular antigen or antigen-binding scaffolds. The antigen binding domains in accordance with the disclosure may recognize and bind a specific antigen or epitope. It should be therefore noted that the term “binding specificity”, “specifically binds to an antigen”, “specifically immuno-reactive with”, “specifically directed against” or “specifically recognizes”, when referring to an antigen or particular epitope, refers to a binding reaction which is determinative of the presence of the epitope in a heterogeneous population of proteins and other biologics. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics. Still further, as indicated above, an “antigen-binding domain” can comprise or consist of an antibody or antigen-binding fragment of an antibody such as single chain variable fragments (scFv). The term “antibody” as used herein, means any antigen-binding molecule or molecular complex comprising at least one complementarity determining region (CDR) that specifically binds to or interacts with a particular antigen or any epitope thereof. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

A typical antibody is composed of two immunoglobulin (Ig) heavy chains and two Ig light chains. In humans, antibodies are encoded by three independent gene loci, namely the immunoglobulin heavy locus (IgH) on chromosome 14, containing the gene segments for the immunoglobulin heavy chain, the immunoglobulin kappa (κ) locus (IgK) on chromosome 2, containing the gene segments for part of the immunoglobulin light chain and the immunoglobulin lambda (λ) locus (IgL) on chromosome 22, containing the gene segments for the immunoglobulin light chain.

Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

Single domain antibodies also known as nanobodies (also known as Camelid single-domain antibodies or VHHs) have previously obtained by immunizing dromedaries, camels, llamas, alpacas, sharks, murine, rabbits and humans).

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

Single chain variable fragments (scFv) comprise the variable regions of the heavy (VH) and light chains (VL) of immunoglobulins, connected with a short linker peptide. Single-chain variable fragments lack the constant Fc region found in complete antibody molecules. Nevertheless, scFv retains the specificity of the original immunoglobulin, despite removal of the constant regions and the introduction of the linker.

The antibody suitable for the present disclosure may also be a bi-specific antibody (or a tri-specific antibody. As indicated above, the antibody suitable for the invention may also be a variable new antigen receptor antibody (V-NAR), as well as any humanized forms thereof.

The second component of the disclosed CAR molecule optionally further expressed by the engineered hematopoietic cells in accordance with the present disclosure, may comprise at least one hinge region, and/or the transmembrane domain.

A Hinge region, or hinge domain as used herein is meant an extracellular flexible structure connecting between the targeting moiety and the T cell plasma membrane. These sequences are generally derived from IgG subclasses (such as IgG1 and IgG4), IgD, CD28 and CD8 domains.

In yet some further embodiments, a “transmembrane region”, or “transmembrane domain (TMD, also referred to herein as TM)”, of the disclosed CAR molecule, is a functional region of a protein that spans the phospholipid bilayer of a biological membrane, such as the plasma membrane of a cell. Integral membrane proteins typically comprise two or more such domains, alternating with intracellular and extracellular domains arranged on either side of the membrane. TMDs may consist predominantly of nonpolar amino acid residues and generally adopt an alpha helix conformation. Amino acids of the transmembrane domains interact with the fatty acyl groups of the membrane phospholipids, thereby anchoring the protein in the membrane.

In some embodiments, the hinge region of the disclosed CAR molecule may comprise any sequence derived from the Cluster of Differentiation 8 α (CD8α) protein, the Cluster of Differentiation 28 (CD28), or the IgG4 hinge region.

Still further, the third component of the CAR molecule optionally expressed by the engineered cell of the T lineage in accordance with the present disclosure is at least one signal transduction domain. As used herein, the term signal transduction domain, refers in some embodiments to the functional, intracellular portion of a receptor protein that acts to transmit the detected stimulatory information within the cell, thereby regulating the cellular activity through specific signaling pathways. According to some embodiments, this domain is an intracellular domain connected to the transmembrane domain.

Still further, in some embodiments, the at least one intracellular T cell signal transduction domain of the of the CAR-molecule optionally expressed by the engineered cell/s of the T lineage in accordance with the present disclosure, comprises at least one tumor necrosis factor (TNF) receptor family member, specifically, the 4-1BB. In yet some further optional embodiments, the at least one intracellular T cell signal transduction domain of the of the CAR-molecule of the present disclosure, further comprises at least one TCR molecule or any fragments thereof. In more specific embodiments, such domain is derived from the cluster of differentiation 3 (CD3) zeta chain or crystallizable fragment receptor gamma (FcRg).

Still further, in some embodiments, the CAR molecule of the present disclosure compromises an intracellular domain derived from the 4-1BB.

4-1BB (TNFRSF9, CD137) is an activation-induced T cell costimulatory molecule, and a TNFR superfamily member. 4-1BB is expressed on a subset of resting CD8⁺ T cells and is upregulated on both CD4⁺ and CD8⁺ T cells following activation. Upon binding to trimeric 4-1BBL (TNFSF9, CD137L) on APCs, 4-1BB recruits TNFR-associated factor family members (TRAF1, TRAF2 and TRAF3) to its cytosolic region, forming the 4-1BB signalosome and leading to downstream activation of NF-κB, MAPK and ERK. Agonistic stimulation of 4-1BB upregulates expression of the anti-apoptotic proteins Bcl-x_{L} and Bfl-1. Still further, 4-1BB activation increases IL-2 and IFN-γ in CD8⁺ cells and IL-2 and IL-4 in CD4⁺ cells. T cells expressing CARs that incorporate 4-1BB domains have been shown to express granzyme B, IFN-γ, TNF-α, GM-CSF and the anti-apoptotic protein Bcl-x_{L}. Still further, incorporation of the 4-1BB TM and cytoplasmic domain into a CAR, leads to improved persistence and antitumor activity, as well as to prolonged T cell division.

Thus, in some embodiments, the CAR molecule optionally expressed by the engineered cell/s of the present disclosure compromises an intracellular domain that comprises the CD3ζ. T-cell surface glycoprotein CD3 zeta chain also known as T-cell receptor T3 zeta chain or CD247 (Cluster of Differentiation 247), a protein encoded in human by the CD247 gene. More specifically, CD3 (cluster of differentiation 3) T-cell co-receptor helps to activate the cytotoxic T-cell. It consists of a protein complex and is composed of four distinct chains. In mammals, the complex contains a CD3γ chain, a CD36 chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex. T-cell receptor zeta, together with T-cell receptor alpha/beta and gamma/delta heterodimers and CD3-gamma, -delta, and -epsilon, forms the T-cell receptor-CD3 complex. The zeta-chain plays an important role in coupling antigen recognition to several intracellular signal-transduction pathways and is thus included in the CAR-T molecule further expressed by the engineered T cell/s of the present disclosure. In some specific and non-limiting embodiments, the engineered cells of the T linage in accordance with the present disclosure may further express the CD19-CAR-T molecule. The CD19 molecule is present in B-cell-derived cancers such as acute lymphoblastic leukemia (ALL) and diffuse large B-cell lymphoma (DLBCL. In some embodiments, the genetically engineered hematopoietic cells, further express the CD19-CAR-T molecule that comprises the amino acid sequence as denoted by SEQ ID NO: 21, and encoded by the nucleic acid sequence as denoted by SEQ ID NO: 22. In yet some further embodiments, the genetically engineered hematopoietic cells, further express the CD19-CAR-T molecule that comprises the amino acid sequence as denoted by SEQ ID NO: 23, and encoded by the nucleic acid sequence as denoted by SEQ ID NO: 24.

Still further, in some embodiments, the engineered hematopoietic cells, specifically, lymphocytes, and more specifically, cells of the T linage in accordance with the present disclosure may further express at least one CD30-CART molecule.

In some embodiments, the engineered cells of the T linage in accordance with the present disclosure may further express at least one CD33-CAR-T molecule.

In some embodiments, the engineered cells of the T linage in accordance with the present disclosure may further express at least one CD123-CAR-T molecule.

In some embodiments, the engineered cells of the T linage in accordance with the present disclosure may further express at least one FLT3-CAR-T molecule.

In some embodiments, the engineered cells of the T linage in accordance with the present disclosure may further express at least one BCMA-CAR-T molecule.

In some embodiments, the engineered cells of the T linage in accordance with the present disclosure may further express at least one CD138-CAR-T molecule.

In yet some further additional or alternative embodiments, the engineered cells of the T linage in accordance with the present disclosure may further express at least one T cell receptor targeted against at least one target antigen. More specifically, the T-cell receptor (TCR) is a protein complex found on the surface of T cells, or T lymphocytes, that is responsible for recognizing fragments of antigen as peptides bound to major histocompatibility complex (MHC) molecules. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors.

The core TCR complex consists of two TCR chains and six cluster of differentiation 3 (CD3) chains. The human genome expresses four TCR genes known as TCRα, TCRβ, TCRγ, and TCRδ, which forms two distinct heterodimers: TCRα/TCRβ or TCRγ/TCRδ. The majority of mature T cells expresses TCRα and TCRβ isoforms, generally referred to as T cells (or αβ T cells), while a small portion (0.5-5%) of T lymphocytes (γδ T cells) expresses TCRγ and TCRδ isoforms. Both heterodimers form multiprotein complexes with CD3 δ, γ, ε, and ζ chains. However, in both complexes, three dimers of CD3 proteins, δε and γε heterodimers and ζζ homodimers, are present. These CD3 proteins associate with TCR via non-covalent hydrophobic interactions and are required for a complete TCR localization on the cell surface. The TCR mediates recognition of antigenic peptides bound to MHC molecules (pMHC), whereas the CD3 molecules transduce activation signals to the T cell. In some embodiments, the engineered hematopoietic cells of the present disclosure may further express a TCR specific for at least one melanoma specific and/or associated antigen. In yet some further embodiments, the disclosed hematopoietic cells of the T lineage may further comprise at least one TCR specific for the MART-1 antigen. In some embodiments, the MART-1 antigen as used herein, refers to the human MART-1, as denoted by Uniprot NP_005502.1. In yet some further embodiments, the human MART-1 antigen is encoded by a sequence comprising the nucleic acid sequence as denoted by genbank accession number CCDS6466.1. still further, in some embodiments, the MART-1 antigen as used herein comprises the amino acid sequence as denoted by SEQ ID NO: 11. Still further, in some embodiments, the MART-1 antigen is encoded by a nucleic acid sequence comprising the sequence as denoted by SEQ ID NO: 12.

In yet some further embodiments, the TCR specific for the MART-1 antigen expressed by the engineered cell of the present disclosure, may comprise an alpha chain comprising the amino acid sequence as denoted by SEQ ID NO; 13. Still further, in some embodiments, the TCR of the present disclosure comprises an alpha chain encoded by the nucleic acid sequence as denoted by SEQ ID NO: 15. Still further, in some embodiments, the anti-MART-1 TCR expressed by the engineered cells of the present disclosure may comprise a beta chain comprising the amino acid sequence as denoted by SEQ ID NO: 14. In some embodiments, such beta chain is encoded by the nucleic acid sequence as denoted by SEQ ID NO: 16. In some embodiments, the TCR is termed by the present disclosure as F4.

In yet some further embodiments, the TCR specific for the MART-1 antigen expressed by the engineered cell of the present disclosure, may comprise an alpha chain comprising the amino acid sequence as denoted by SEQ ID NO: 17. Still further, in some embodiments, the TCR of the present disclosure comprises an alpha chain encoded by the nucleic acid sequence as denoted by SEQ ID NO: 19. Still further, in some embodiments, the anti-MART-1 TCR expressed by the engineered cells of the present disclosure may comprise a beta chain comprising the amino acid sequence as denoted by SEQ ID NO: 18. In some embodiments, such beta chain is encoded by the nucleic acid sequence as denoted by SEQ ID NO: 20. In some embodiments, the TCR is termed by the present disclosure as F5. It should be further appreciated that the engineered hematopoietic cells of the present disclosure may be genetically edited to express the optional receptor molecule (e.g., the CAR and/or TCR) as described herein, together with the at least one molecule involved directly or indirectly in at least one metabolic pathway, for example, at least one of HK2, PFK-1, GLUT3, PK-M, and/or any combination thereof. The nucleic acid sequences encoding the receptors and/or the metabolic pathway proteins may be provided either in separate construct, vector, and/or cassette, and may be genetically edited either together or separately. Alternatively, the nucleic acid sequences encoding the receptors and/or the metabolic pathway proteins may be provided in the same construct, vector, and/or cassette.

In some embodiments, the target antigen targeted by the at least one receptor molecule further expressed by the genetically engineered cell/s of the present disclosure, may be an antigen associated with a pathologic disorder.

In some specific and non-limiting embodiments, the genetically engineered cell/s of the present disclosure further expresses at least one CAR molecule that comprises as the antigen-binding component thereof, an anti-CD19 scFv.

In yet some further embodiments, CAR molecules expressed by the genetically engineered cell/s of the present disclosure, may be CARs targeting many other blood cancer antigens, including CD30 in refractory Hodgkin's lymphoma; CD33, CD123, FLT3, in acute myeloid leukemia (AML); and BCMA in multiple myeloma.

In yet some further embodiments, CAR molecules expressed by the genetically engineered cell/s of the present disclosure, may be CARs targeting any antigen associated with any solid or no-solid tumor as disclosed by the present disclosure, for example, melanoma.

In some embodiments, the at least one target antigen targeted by the TCR or CAR molecules further expressed by the genetically engineered cell/s of the present disclosure, may be at least one of: at least one tumor associated antigen (TAA), at least one tumor specific antigen (TSA), at least one neoantigen, at least one viral antigen, at least one bacterial antigen, at least one fungal antigen and/or at least one parasite antigen.

It should be understood that the at least one receptor molecule comprising the at least one target binding domain that may be directed to any antigen of interest, specifically any antigen specific for a pathologic disorder. In more specific embodiments, the receptor molecule expressed either endogenously or exogenously by the engineered T cell, may be directed against antigens specific for proliferative disorders, specifically, tumor associated antigens (TAAs), tumor specific antigen (TSA), or antigens specific for any pathogen, specifically, viral, bacterial, fungal or parasitic pathogen. Specific pathogens applicable in the present disclosure, are described in more detail herein after.

In some specific embodiments, the at least one receptor molecule disclosed in the disclosure, may comprise at least one antibody directed against at least one tumor associated antigen (TAA).

Tumor or cancer associated antigen (TAA), as used herein may be an antigen that is specifically expressed, over expressed or differentially expressed in tumor cells. In yet some further embodiments, TAA can stimulate tumor-specific T-cell immune responses. Exemplary tumor antigens that may be applicable in the present disclosure, include, but are not limited to, Melan-A/MART-1, RAGE-1, tyrosinase, MAGE-1, MAGE-2, NY-ESO-1, glycoprotein (gp) 75, gp100, MUC1, beta-catenin, PRAME, MUM-1, WT-1, CEA, PR-1 CD45, glypican-3, IGF2B3, Kallikrein4, KIF20A, Lengsin, Meloe, MUC5AC, survivin, CLPP, Cyclin-A1, SSX2, XAGElb/GAGED2a, MAGE-A3, MAGE-A6, LAGE-1, CAMEL, hTRT and Eph, and TRP-1. Still further, TAA may be recognized by CD8+ T cells as well as CD4+ T cells. Non limiting examples of TAA recognized by CD8+ T cells may be CSNKIAI, GAS7, HAUS3, PLEKHM2, PPPIR3B, MATN2, CDK2, SRPX (P55L), WDR46 (T2271), AHNAK (54460F), COL18A1 (S126F), ERBB2 (H197Y), TEADI (L209F), NSDHL (A290V), GANAB (S184F), TRIP12 (F1544S), TKT (R438W), CDKN2A (E153K), TMEM48 (F169L), AKAP13 (Q285K), SEC24A (P469L), OR8B3 (T190I), EXOC8 (Q656P), MRPS5 (P59L), PABPC1 (R520Q), MLL2, ASTN1, CDK4, GNL3L, SMARCD3, MAGE-A6, MED13, PAS5A WDR46, HELZ2, AFMID, CENPL, PRDX3, FLNA, KIF16B, SON, MTFR2 (D626Y), CHTF18 (L769V), MYADM (R30W), NUP98 (A359D), KRAS (G12D), CASP8 (F67V), TUBGCP2 (P293L), RNF213 (N1702S), SKIV2L (R653H), H3F3B (A48T), AP15 (R243Q), RNF10 (E572K), PHLPP1 (G566E) and ZFYVE27 (R6H). Non limiting examples of TAA recognized by CD4+ T cells may be ERBB2IP (E805G), CIRHIA (P333L), GART (V551A), ASAP1 (P941L), RND3 (P49S), LEMD2 (P495L), TNIK (S502F), RPS12 (V104I), ZC3H18 (G269R), GPD2 (E426K), PLEC (E1179K), XPO7 (P274S), AKAP2 (Q418K) and ITGB4 (S10021). Non-limiting examples of MHC class II-restricted antigens may be Tyrosinase, gp100, MART-1, MAGE-A1, MAGE-A2, MAGE-A3, MAGE-A6, LAGE-1, CAMEL, NY-ESO-1, hTRT and Eph. In some embodiments, the TAA may be CD30 (in refractory Hodgkin's lymphoma). In yet some further embodiments, the TAA may be CD33. In some embodiments, the TAA may be CD123. In some embodiments, the TAA may be FLT3 that is expressed in in acute myeloid leukemia (AML). In some embodiments, the TAA may be BCMA that is expressed in multiple myeloma. In some embodiments, the TAA may be MESOTHELIN that is expressed in lung, pancreatic cancer. In some embodiments, the TAA may be GD2 that is expressed in melanoma, brain, pancreatic cancers. In some embodiments, the TAA may be CEA that is expressed in Colorectal cancer. In some embodiments, the TAA may be HER2 that is expressed in in breast cancer. In some embodiments, the TAA may be CEACAM 7 that is expressed in Pancreatic cancer. In some embodiments, the TAA may be CD22 expressed in Lymphoma. In some embodiments, the TAA may be mutated p53. Still further in some embodiments, the TAA may be gp100 that is expressed in Melanoma.

In some embodiments, the TAA may be Tyrosinase (Melanoma). In some embodiments, the TAA may be NY-ESO1 (Epithelial malginancies). In some embodiments, the TAA may be MAGE antigen family. In some embodiments, the TAA may be WT1 (hematological malignancies). In some embodiments, the TAA may be MUC-1 (Breast ovarian carcinoma, pancreatic cancer).

In some embodiments, the TAA may be LMP2 (EBV-linked malignancies). In some embodiments, the TAA may be E6, E7 (HPV-linked malignancies).

Cancer antigen and tumor antigen are used interchangeably herein. The antigens may be related to cancers that include, but are not limited to, skin cancer, hematological malignancies, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (e.g., nonmelanoma, melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

Still further, in some embodiments, a few examples of antibodies used in the treatment of cancer that may be applicable in the present disclosure include, but are not limited to monoclonal antibodies such as Bevacizumab (UNII: 2S9ZZM9Q9V), Cetuximab (UNIT: PQX0D8J21J), Panitumumab (UNII: 6A901E312A), Rituximab (UNII: 4F4X42SYQ6), Alemtuzumab (UNII: 3A189DH42V), Trastuzumab (UNII: P188ANX8CKO, that is directed against HER2, Ipilimumab (UNII: 6T8C155666, Yervoy), that is a check point inhibitor, specifically, a monoclonal antibody that works to activate the immune system by targeting CTLA-4, Tremelimumab, formerly ticilimumab (CP-675,206) is a fully human monoclonal antibody against CTLA-4, ibritumomab tiuxetan (UNII: 4Q52C550XK), lambrolizumab (formerly MK-3475, Pembrolizumab, Keytruda® UNII: DPT003T46P), that is a check point inhibitor, specifically, a humanized antibody that targets programmed cell death (PD-1), Nivolumab (Opdivo® UNII: 31YO63LBSN) is an Fab fragment of an antibody that binds the extracellular domain of PD-1, Atezolizumab (trade name Tecentriq) is a fully humanized, engineered monoclonal antibody of IgG1 isotype against the protein programmed cell death-ligand 1 (PD-L1), Avelumab (trade name Bavencio) is a fully human monoclonal antibody that targets PD-L1, Durvalumab is a human immunoglobulin G1 kappa (IgG1K) monoclonal antibody that blocks the interaction of PD-L1 with the PD-1 and CD80 (B7.1) molecules and Tremelimumab (formerly ticilimumab; UNII: QEN1X95CIX) that is a check point inhibitor and ado-trastuzumab emtansine (UNII: SE2KH7T06F).

In some embodiments, the at least one targe antigen recognized by the receptor further expressed by the hematopoietic engineered cells, may be a tumor specific antigen.

A tumor-specific antigen (TSA) is a protein or other molecule that is produced by cancer cells and not normally found in healthy cells. There are different types of TSAs, including those that are specific to a particular type of cancer (such as prostate-specific antigen for prostate cancer) and those that are more broadly expressed across different types of cancer (such as carcinoembryonic antigen). Major classes of TSAs, include those generated from mutational frameshifts, splice variants, gene fusions, endogenous retroelements and other classes, such as human leukocyte antigen (HLA)-somatic mutation-derived antigens and post-translational TSAs. Non-limiting examples for TSAs useful in the present disclosure may include, but are not limited to mutated p53, mutated Ras, various splice variants (e.g., RHAMM-48 and RHAMM-147), BCR-ABL fusion and the like. In some particular embodiments, receptors optionally expressed in the genetically engineered cells of the present disclosure may be directed against any antigen derived from a pathogen, specifically, viral, bacterial, fungal, parasitic pathogen and the like. Thus, in some specific embodiments, the at least one receptor expressed by the genetically engineered cell of the T lineage, may be directed against a viral antigen. It should be appreciated that any of the viral pathogens discussed herein after, is applicable in this aspect, as well as in all aspects of the present disclosure.

In some specific embodiments, the viral pathogen may be of any of the following orders, specifically, Herpesvirales (large eukaryotic dsDNA viruses), Ligamenvirales (linear, dsDNA (group I) archaean viruses), Mononegavirales (include nonsegmented (−) strand ssRNA (Group V) plant and animal viruses), Nidovirales (composed of (+) strand ssRNA (Group IV) viruses), Ortervirales (single-stranded RNA and DNA viruses that replicate through a DNA intermediate (Groups VI and VII)), Picornavirales (small (+) strand ssRNA viruses that infect a variety of plant, insect and animal hosts), Tymovirales (monopartite (+) ssRNA viruses), Bunyavirales contain tripartite (−) ssRNA viruses (Group V) and Caudovirales (tailed dsDNA (group I) bacteriophages).

In yet some further specific embodiments, the receptors optionally expressed in the genetically engineered cells the present disclosure may be specifically directed against DNA viruses, specifically, any virus of the following families: the Adenoviridae family, the Papovaviridae family, the Parvoviridae family, the Herpesviridae family, the Poxviridae family, the Hepadnaviridae family and the Anelloviridae family.

In yet some further specific embodiments, the receptors optionally expressed in the genetically engineered cells may be specifically directed against RNA viruses, specifically, any virus of the following families: the Reoviridae family, Picornaviridae family, Caliciviridae family, Togaviridae family, Arenaviridae family, Flaviviridae family, Orthomyxoviridae family, Paramyxoviridae family, Bunyaviridae family, Rhabdoviridae family, Filoviridae family, Coronaviridae family, Astroviridae family, Bornaviridae family, Arteriviridae family, Hepeviridae family and the Retroviridae family.

In more specific embodiments, the receptors optionally expressed in the genetically engineered cells may be directed against any antigen derived from a viral pathogen of the order Mononegavirales. In yet some further embodiments, receptors optionally expressed in the genetically engineered cells may be directed against an antigen derived from a virus of the family Pneumoviridae. In more specific embodiments, the receptors optionally expressed in the genetically engineered cells may be directed against any antigen derived from a viral pathogen of the genus Orthopneumovirus. In some specific embodiments, such viral antigen may be an antigen specific for respiratory syncytial virus (RSV), for example, any one of the Human respiratory syncytial virus (HRSV), A2 and B1, the bovine respiratory syncytial virus (BRSV) and the murine pneumonia virus (MPV). In more specific embodiments, the receptors optionally expressed in the genetically engineered cells may be directed against the human RSV. In yet some further specific embodiments, the anti-RSV antibody may be the anti-RSV palivizumab antibody. More specifically, Palivizumab (brand name Synagis, manufactured by MedImmune) is a humanized monoclonal antibody (IgG) directed against an epitope in the A antigenic site of the F protein of RSV.

In yet some further specific embodiments, the receptors optionally expressed by the genetically engineered cells may be directed against any antigen derived from a viral pathogen of the family Retroviridae. In yet some further embodiments, the receptors optionally expressed in the genetically engineered cells may be directed against an antigen derived from a virus of the subfamily Orthoretrovirinae. In more specific embodiments receptors optionally expressed in the genetically engineered cells may be directed against any antigen derived from a viral pathogen of the genus Lentivirus, specifically, of the species human immunodeficiency virus (HIV).

Specific embodiments that relate to particular viruses associated with specific disorders are specified herein below. It should be understood that any of the viral pathogens and any of the bacterial, fungal and parasite pathogen described herein after, are also applicable in connection with the antigens derived therefrom that are recognized by the receptors optionally expressed in the genetically engineered cells of the present disclosure.

In more specific embodiments, the receptors optionally expressed in the genetically engineered cells may be directed against at least one neoantigen. A neoantigen, as used herein, is a type of antigen created by a genetic mutation, alternative splicing or other alteration in a cell, for example, a cancer cell. Neoantigens are not present in normal, healthy cells, making them an attractive target for cancer immunotherapy. Neoantigens are formed when a mutation occurs in regulatory or encoding sequences leading to the creation of a novel protein or a modification of an existing protein. The mutated protein is then presented on the surface of the cell as an antigen, which can be recognized by the immune system as foreign.

In some specific embodiments, the antigen recognized by the TCR or CAR molecule that is further expressed by the genetically engineered cell/s of the present disclosure, is a melanoma antigen. In yet some further embodiments, the melanoma antigen may be the MART-1 antigen as disclosed herein above.

In some embodiments of the disclosed cell, the at least one intracellular T cell signal transduction domain of the CAR molecule, may comprise at least one domain of tumor necrosis factor (TNF) receptor family member, and optionally, at least one domain of a T cell receptor (TCR) molecule. Specific examples for CAR and/or TCR molecules applicable in the present disclosure are disclosed herein above.

In some embodiments, the signaling domain of the CAR molecule that is further expressed by the genetically engineered cell/s of the present disclosure, may be at least one costimulatory signaling domain selected from the group of 4-1BB, a cluster of differentiation 28 (CD28), and/or a cluster of differentiation 3 (CD3) zeta chain.

In some embodiments, the at least one protein involved directly or indirectly in at least one metabolic pathway expressed by the at least one genetically engineered cell of the T lineage of the present disclosure, may result in at least one of: (i) increased cytokine secretion; (ii) increased expression of activation markers; (iii) increased glycolysis; (iv) improvement in metabolic parameters; (v) increased cell survival; (vi) increased cytotoxicity; (vii) reduced expression of exhaustion markers; (viii) increase in oxidative phosphorylation; and/or (ix) increased proliferation, by the genetically engineered hematopoietic cells, specifically, lymphocytes, and more specifically, cell of the T lineage.

The disclosed engineered cells of the T lineage display increased activation. As indicated above, increased activation of T cells may be reflected for example, by the increase in the expression of activation markers. Activation markers refer herein to molecules which are upregulated upon T cell activation, each at a different stage of the activation process. For example, the earliest activation marker is CD69, which is an inducible cell surface glycoprotein expressed upon activation via the TCR or the IL-2 receptor (CD25). It plays a role in the proliferation and survival of activated T lymphocytes.

According to some embodiments, increase in the expression of activation markers, may comprise for example, increase in the expression of at least one of Leukocyte antigen 37 (CD37), CD25 (also known as the α chain of the high affinity IL-2 receptor), Cluster of differentiation 69 (CD69), as compared with control cells, specifically cells that are not genetically engineered to express the at least one molecule involved directly or indirectly in at least one metabolic pathway, for example, at least one of HK2, PFK-1, GLUT3, PK-M, and/or any combination thereof.

In yet some further embodiments, the engineered cell/s of the T lineage provided by the present disclosure display reduced exhaustion. This reduced exhaustion is reflected by reduced expression of exhaustion markers in response to a specific stimulation. T cell Exhaustion as used herein refers to a state of T cell dysfunction that arises during many chronic infections and cancer. It is defined by poor effector function, sustained expression of inhibitory receptors and a transcriptional state distinct from that of functional effector or memory T cells. By “dysfunction” here it is understood that some T cells, after activation and proliferation, do not fulfill the functions they are expected to perform as effector T cells-typically, they fail to eliminate cancerous or infected cells and control the tumor or the virus respectfully. As originally described, antigen-specific T cells become “dysfunctional” during the chronic phase of high viral load infections, with progressive loss of interleukin (IL)-2, then tumor necrosis factor alpha (TNFα), and, finally, interferon gamma (IFNγ). Thus, in some embodiments, the disclosed engineered cells of the T lineage display, or characterized by reduced expression of exhaustion markers. More specifically, in some embodiments, the exhaustion markers may be at least one of Programmed Death-1 receptor (PD-1), Lymphocyte activation gene-3 (LAG-3, is also named CD223 or FDC protein), T-cell immunoglobulin and mucin-domain containing-3 (TIM3), T cell immunoreceptor with Ig and ITIM domains (TIGIT). Still further, in some additional and non-limiting embodiments, exhaustion markers applicable in the present disclosure include inducible T-cell co-stimulator (ICOS), cytotoxic T-lymphocyte-associated protein-4 (CTLA-4), CD244 (2B4), CD160, killer cell lectin-like receptor subfamily G member 1 (KLRG1), and the like. In some embodiments, the engineered cells displaying reduced expression of at least one exhaustion markers as disclosed herein, as compared with control cells, specifically cells that are not genetically engineered to express the at least one molecule involved directly or indirectly in at least one metabolic pathway, for example, at least one of HK2, PFK-1, GLUT3, PK-M, and/or any combination thereof.

In some embodiments, the indicated results of the expression of the at least one protein by the disclosed genetically engineered cell may be in response to a specific stimulation in an in vivo and/or in vitro/ex vivo setting, for example, activation of T-cells (e.g., by anti-CD3 antibodies and the like.

In yet some further embodiments, the engineered cells display increased cytokine secretion. Cytokine secretion refers to the process by which cells release cytokines into the surrounding tissue or media. Cytokines, in accordance with the present disclosure include interleukins, for example, interleukine-2, (IL-2), interleukin-1 (IL-1) and interleukin-6 (IL-6), interferon (IFN), Tumor necrosis factor (TNF), Chemokines, such as Transforming growth factor-beta (TGF-beta) and the like.

Still further, in some embodiments, the engineered cells display increased cell survival.

Increased cell survival refers to the ability of cells to withstand stress or damage and continue to survive and function normally. This can be achieved through various mechanisms, such as increased resistance to oxidative stress, DNA damage, or other forms of cellular stress, as well as enhanced repair mechanisms that allow damaged cells to recover and continue to function.

In yet some further embodiments, the engineered cells display increased cytotoxicity. Increased T cell cytotoxicity refers to the ability of T cells to efficiently target and eliminate specific target cells. Cytotoxic T cells (also known as CD8+ T cells) recognize and bind to specific antigens presented on the surface of target cells and then release cytotoxic molecules, such as perforin and granzyme, to induce cell death. Increased T cell cytotoxicity can be achieved through various mechanisms, such as upregulation of cytotoxic molecules, increased expression of activation markers, or enhanced differentiation and proliferation of T cells. In yet some further embodiments, the engineered cells display increased oxidative phosphorylation. Increased An increase in oxidative phosphorylation refers to an increase in the metabolic process by which cells convert nutrients, such as glucose and fatty acids, into usable energy in the form of ATP (adenosine triphosphate). This process occurs in the mitochondria of cells and involves a series of enzymatic reactions, collectively known as the electron transport chain, which utilize oxygen to generate a proton gradient across the inner mitochondrial membrane.

In yet some further embodiments, the engineered cells display increased cell proliferation. Increased cell proliferation refers to an increase in the rate at which cells divide and multiply to produce new cells. Cell proliferation is regulated by a complex network of signaling pathways and checkpoints that ensure proper cell cycle progression and prevent uncontrolled growth.

In some embodiments, the improvement in metabolic parameters displayed by the genetically engineered cell/s of the present disclosure, may comprise at least one of: increased ATP content, glucose uptake and/or mitochondrial mass.

The disclosed engineered hematopoietic cells, specifically, lymphocytes, and more specifically, cells of the T-lineage display in some embodiments increased and elevated activity that is indicated in several functional and structural parameters. Increase, as used herein, in connection with various improved properties of the T-cell of the present disclosure, is meant that such increase or enhancement may be an increase or elevation or improvement of the indicated feature/activity (e.g., cytokine secretion, expression of activation markers, survival, proliferation, and the like), of between about 1% to 100%, specifically, 5% to 100% of the indicated parameter, more specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more, as compared with control cells, specifically cells that are not genetically engineered to express the at least one molecule involved directly or indirectly in at least one metabolic pathway, for example, at least one of HK2, PFK-1, GLUT3, PK-M, and/or any combination thereof. In yet some further embodiments, the terms “inhibition”, “moderation”, “reduction”, “decrease” or “attenuation” as referred to herein with respect to the various properties of the T-cell of the present disclosure (e.g., expression of exhaustion markers), relate to the retardation, restraining or reduction of the indicated parameter by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. More specifically, the terms “increase”, “augmentation” and “enhancement” as used herein relate to the act of becoming progressively greater in size, amount, number, or intensity. Alternatively, “inhibition”, “moderation”, “reduction”, “decrease” or “attenuation” as used herein relate to the act of becoming progressively smaller in size, amount, number, or intensity. Particularly, an increase or alternatively, decrease of 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the indicated activity (e.g., increase of cytokine secretion and/or expression of activation markers, or alternatively, decrease of expression of exhaustion markers), as compared to a suitable control, e.g., cells that are not genetically engineered to express the at least one molecule involved directly or indirectly in at least one metabolic pathway, for example, at least one of HK2, PFK-1, GLUT3, PK-M, and/or any combination thereof.

In some embodiments, the genetically engineered hematopoietic cell of the present disclosure, specifically, a cell of the T lineage in accordance with the disclosed specification, may be a cell genetically engineered by at least one nucleic acid sequence encoding the at least one molecule involved directly or indirectly in at least one metabolic pathway, or any cassette, vector and/or gene editing system comprising the nucleic acid sequence. It is therefore appreciated that the present disclosure further encompasses any vector, cassette, construct and/or plasmid comprising nucleic acid sequence/s encoding at least one of the disclosed molecules involved in at least one metabolic pathway, for example, at least one of HK2, PFK-1, GLUT3, PK-M, and/or any variant and isoform thereof, and any combination thereof.

In yet some further specific embodiments, the present disclosure provides at least one hematopoietic cell, for example, a cell of the T lineage, that comprises and/or is genetically edited by at least one construct and/or cassette comprising the nucleic acid sequence encoding the HK protein. In some embodiments, such cassette may comprise the nucleic acid sequence as denoted by SEQ ID NO: 25, or any derivatives and variants thereof, or any codon optimized version thereof. Still further, in some further specific embodiments, the present disclosure provides at least one hematopoietic cell, for example, a cell of the T lineage, that comprises and/or is genetically edited by at least one construct and/or cassette comprising the nucleic acid sequence encoding the PFK protein.

In some embodiments, such cassette may comprise the nucleic acid sequence as denoted by SEQ ID NO: 28, or any derivatives and variants thereof, or any codon optimized version thereof. In some embodiments, the present disclosure further provides at least one engineered cell, specifically, cell of the T lineage that comprises and/or is genetically edited by at least one construct and/or cassette comprising the nucleic acid sequence encoding the PFK protein, in a construct comprising PFK-IRES-NGFR. In some embodiments, such cassette may comprise the nucleic acid sequence as denoted by SEQ ID NO: 41, or any derivatives and variants thereof, or any codon optimized version thereof.

In some further embodiments, the present disclosure further provides at least one engineered cell, specifically, cell of the T lineage that comprises and/or is genetically edited by at least one construct and/or cassette comprising the nucleic acid sequence encoding the PFK and the GLUT3 proteins, optionally, separated by at least one IRES sequence. In some embodiments, such cassette may comprise the nucleic acid sequence as denoted by SEQ ID NO: 26, or any derivatives and variants thereof, or any codon optimized version thereof. Still further, in some embodiments, the present disclosure provides at least one engineered cell, specifically, cell of the T lineage that comprises and/or is genetically edited by at least one construct and/or cassette comprising the nucleic acid sequence encoding the GLUT3 protein. In some embodiments, such cassette may comprise the nucleic acid sequence as denoted by SEQ ID NO: 6, or any derivatives and variants thereof, or any codon optimized version thereof. In some embodiments, the present disclosure further provides at least one engineered cell, specifically, cell of the T lineage that comprises and/or is genetically edited by at least one construct and/or cassette comprising the nucleic acid sequence encoding the GLUT3 protein, in a construct comprising GLUT3-IRES-NGFR. In some embodiments, such cassette may comprise the nucleic acid sequence as denoted by SEQ ID NO: 40, or any derivatives and variants thereof, or any codon optimized version thereof.

In yet some further specific embodiments, the present disclosure provides at least one hematopoietic cell, for example, a cell of the T lineage, that comprises and/or is genetically edited by at least one construct and/or cassette comprising the nucleic acid sequence encoding the PKM protein. In some embodiments, such cassette may comprise the nucleic acid sequence as denoted by SEQ ID NO: 27, or any derivatives and variants thereof, or any codon optimized version thereof. Still further, in some embodiments, the present disclosure provides at least one engineered hematopoietic cell (specifically, of the T lineage) that comprises and/or is genetically edited by at least one construct and/or cassette comprising the nucleic acid sequence encoding any combinations of the disclosed HK, PFK, GLUT3, PKM proteins, and/or any combinations thereof with sequences encoding the NGFR protein, or any parts thereof, for example, the nucleic acid sequence as denoted by SEQ ID NO: 10, or any derivatives and variants thereof, that encodes the truncated NGFR as denoted by SEQ ID NO: 39, or any codon optimized version thereof. Still further, it should be appreciated that the present disclosure encompasses any engineered hematopoietic cells, specifically, cell of the T lineage, that comprise and/or is edited by the nucleic acid sequences as disclosed above, and/or further express any of the disclosed receptors, specifically, the TCRs and/or the CAR molecules disclosed above. More specifically, the present disclosure encompasses any cell expressing and/or engineered by the nucleic acid sequence as denoted by at least one of SEQ ID NOs: 15 and 16, 19 and 20, encoding alpha and beta chains of the MART-1 specific TCRs (F4, F5), and/or the CAR molecules encoded by SEQ ID NO:22 and SEQ ID NO:24, or any codon optimized version thereof.

Still further, it should be appreciated that the present disclosure further encompasses any of the nucleic acid sequences as disclosed herein, specifically, any nucleic acid sequence comprising the nucleic acid sequence of any one of SEQ ID NOs: 15, 16, 19, 20, 22, 24, 25, 26, 27, 28, 10, 6, 40, 41, or any codon optimized version thereof, and/or any combinations thereof, any cassette, vector, or delivery vehicle comprising the same. Specific expression vectors, and vehicles applicable in the present disclosure are described herein after in connection with other aspects of the present disclosure.

The present disclosure provides at least one molecule involved directly or indirectly in at least one metabolic pathway, and optionally, in combination with at least one receptor, for example, TCR, or CAR-T molecules that are composed of amino acid residues and are therefore a polypeptide. The term “polypeptide” as used herein refers to amino acid residues, connected by peptide bonds. A polypeptide sequence is generally reported from the N-terminal end containing free amino group to the C-terminal end containing free carboxyl group and may include any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. More specifically, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof. Proteins orthologs or homologues having a sequence homology or identity to the proteins of interest in accordance with the disclosure, specifically, receptors, chimeras and antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest.

In some embodiments, derivatives refer to polypeptides, which differ from the polypeptides specifically defined in the present disclosure by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the disclosure as indicated above, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the disclosure may occur in any position of the modified peptide, as well as in any of the N′ or C′ termini thereof. With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the disclosure. For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the polypeptides of the disclosure may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the disclosure.

A further aspect of the present disclosure relates to a composition comprising at least one of: (a), at least one genetically engineered hematopoietic cell/s or a cell population comprising at least one of the genetically engineered cell/s. The genetically engineered cell of the disclosed compositions comprise and/or expresses (e.g., exogenously) at least one nucleic acid sequence encoding at least one molecule involved directly or indirectly in at least one metabolic pathway. The cells of the disclose composition optionally further expresses at least one receptor molecule. In yet some alternative or additional embodiments, the disclosed composition may further comprise and/or alternatively comprise (b), at least one nucleic acid sequence encoding the at least one molecule involved directly or indirectly in at least one metabolic pathway, or any cassette, vector and/or gene editing system comprising the nucleic acid sequence. In some embodiments, the composition further comprises at least one of pharmaceutically acceptable carrier/s, diluent/s, excipient/s and additive/s. In some embodiments, the genetically engineered hematopoietic cells may be any lymphocyte cells. In yet some further embodiments, the genetically engineered lymphocytes of the present disclosure may be genetically engineered cells of the T cell lineage.

In some embodiments, the genetically engineered cell of the T lineage of the disclosed composition may be any of the cells as defined by the present disclosure.

The pharmaceutical compositions of the disclosure can be administered and dosed by the methods of the disclosure, in accordance with good medical practice, systemically, for example by parenteral, e.g., intrathymic, into the bone marrow and intravenous. It should be noted however that the disclosure may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g., oral, intranasal, or intraocular administration.

Local administration to the area in need of treatment may be achieved by, for example, by local infusion during surgery, topical application, direct injection into the specific organ (bone marrow, spleen, lymph nodes), etc. More specifically, the compositions used in any of the methods of the disclosure, described herein, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

More specifically, pharmaceutical compositions used to treat subjects in need thereof according to the disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, nucleic acid molecule/s of the disclosure or any cassette/s thereof, with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present disclosure also include, but are not limited to, emulsions and liposome-containing formulations. It should be understood that in addition to the ingredients particularly mentioned above, the formulations may also include other agents conventional in the art having regard to the type of formulation in question. Still further, pharmaceutical preparations are compositions that include one or more nucleic acid molecules, vectors and/or cassette and/or cells of the present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle”, when referred to the compositions in the present aspect, refers to a diluent, adjuvant, excipient, or carrier with which a compound of the disclosure is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the nucleic acid molecule/s encoding the at least one molecule involved directly or indirectly in at least one metabolic pathway and optionally, the at least one receptor molecule of the disclosure or any engineered cells of the T-lineage, and systems of the disclosure, can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity, or it may be formulated for sustained release.

In numerous embodiments, the compositions of the present disclosure may be administered in a form of combination therapy, i.e. in combination with one or more additional therapeutic agents. Combination therapy may include administration of a single pharmaceutical dosage formulation comprising at least one composition of the disclosure and additional therapeutics agent(s); as well as administration of at least one composition of the disclosure and one or more additional agent(s) in its own separate pharmaceutical dosage formulation. Further, where separate dosage formulations are used, compositions of the disclosure and one or more additional agents can be administered concurrently or at separately staggered times, i.e. sequentially. Still further, said concurrent or separate administrations may be carried out by the same or different administration routes. Thus, in some further embodiments, the engineered cells of the present disclosure may be applicable in boosting the immune response of a subject suffering from a pathologic disorder, and may be used in combined treatment with any therapeutic agent, for example, a chemotherapeutic agent.

As used herein, a “chemotherapeutic agent” or “chemotherapeutic drug” (also termed chemotherapy) as used herein refers to a drug treatment intended for eliminating or destructing (killing) cancer cells or cells of any other proliferative disorder. The mechanism underlying the activity of some chemotherapeutic drugs is based on destructing rapidly dividing cells, as many cancer cells grow and multiply more rapidly than normal cells. As a result of their mode of activity, chemotherapeutic agents also harm cells that rapidly divide under normal circumstances, for example bone marrow cells, digestive tract cells, and hair follicles. Insulting or damaging normal cells result in the common side-effects of chemotherapy: myelosuppression (decreased production of blood cells, hence also immuno-suppression), mucositis (inflammation of the lining of the digestive tract), and alopecia (hair loss).

Various different types of chemotherapeutic drugs are available. A chemotherapeutic drug may be used alone or in combination with another chemotherapeutic drug or with other forms of cancer therapy, in addition to the genetically engineered cell of the present disclosure, for example, other biological drugs (antibodies, ligands, receptors), radiation therapy or surgery.

Chemotherapeutic drugs affect cell division or DNA synthesis and function and can be generally classified into several groups, based on their structure or biological function. More specifically, chemotherapeutic agents that are classified as alkylating agents, anti-metabolites, anthracyclines, plant alkaloids, topoisomerase inhibitors, and other anti-tumor agents such as DNA-alkylating agents, anti-tumor antibiotic agents, tubulin stabilizing agents, tubulin destabilizing agents, hormone antagonist agents, protein kinase inhibitors, HMG-CoA inhibitors, CDK inhibitors, cyclin inhibitors, caspase inhibitors, metalloproteinase inhibitors, antisense nucleic acids, triple-helix DNAs, nucleic acids aptamers, and molecularly-modified viral, bacterial or exotoxic agents. It should be appreciated that any combination therapy disclosed herein, using any of the indicated compounds with the cells and compositions of the present disclosure, together with any of the therapeutic agents discussed above, is encompassed by the present disclosure.

A further aspect of the present disclosure relates to a method for treating, preventing, ameliorating, inhibiting or delaying the onset of a pathologic disorder in a mammalian subject. The methods disclosed herein comprise the step of administering to the subject an effective amount of at least one of: (a), at least one genetically engineered hematopoietic cell/s or a cell population comprising at least one of the genetically engineered cell/s. The genetically engineered hematopoietic cell/s used by the disclosed methods, comprises and/or expresses (e.g., exogenously) at least one nucleic acid sequence encoding at least one molecule involved directly or indirectly in at least one metabolic pathway. The cell/s used by the disclosed methods may optionally further expresses (either exogenously or endogenously, at least one receptor molecule. In yet some further embodiments, the methods disclosed herein may further, or alternatively administer (b), at least one nucleic acid sequence encoding the at least one molecule involved directly or indirectly in at least one metabolic pathway, or any cassette, vector and/or gene editing system comprising the nucleic acid sequence. Still further, the disclosed methods may further, or alternatively administer (c), a composition comprising the genetically engineered cell/s of (a) and/or the nucleic acid sequence of (b). In some embodiments, the genetically engineered hematopoietic cells may be any lymphocyte cells. In yet some further embodiments, the genetically engineered lymphocytes of the present disclosure may be genetically engineered cells of the T cell lineage.

In some embodiments the molecule involved directly or indirectly in at least one metabolic pathway expressed by the engineered cell used by the disclosed therapeutic methods, may be a catabolic protein (e.g., break down complex molecules and release energy in the process), an anabolic protein (e.g., synthesize molecules with the utilization of energy) or an amphibolic protein (e.g., a protein that can be either catabolic or anabolic based on the need for or the availability of energy).

In some embodiments, the engineered cell/s used by the disclosed therapeutic methods expresses exogenously at least one molecule, specifically a protein, involved directly or indirectly in at least one metabolic pathway. In some embodiments, such metabolic pathway may be at least one of: glycolysis pathway, pentose phosphate pathway, fatty acid biosynthesis pathway, electron transport chain, and/or oxidative phosphorylation.

In some embodiments, the protein involved directly or indirectly the at least one metabolic pathway, expressed by the engineered cell/s used by the disclosed therapeutic methods, may be at least one of an enzymatic protein, a transporter protein, a structural protein, an adaptor protein and a protein participating in signal transduction related to the metabolic pathway.

In some embodiments, the protein involved directly or indirectly in at least one metabolic pathway, expressed by the engineered cell/s used by the disclosed therapeutic methods may be at least one of HK, GLUT3, PFK-1, PKM, PGI, ALDO, TPI, GAPDH, PGK, PGM, and/or enolase.

Still further, in some embodiments, the engineered cell used by the disclosed therapeutic methods express at least one protein involved directly or indirectly in at least one metabolic pathway. Such protein according to some embodiments, may be at least one of HK2, PFK-1, GLUT3, PKM, and/or any combination thereof.

In yet some further embodiments, the genetically engineered cell/s used by the disclosed therapeutic methods may further express exogenously or endogenously, at least one receptor molecule comprising at least one target binding domain specific against at least one target antigen.

In some embodiments, at least one receptor further expressed by the genetically engineered cell/s used by the disclosed therapeutic methods, may be at least one TCR molecule, specifically directed at a target antigen.

In some embodiments, the genetically engineered cell/s used by the disclosed therapeutic methods may further express exogenously or endogenously at least one receptor molecule, such as a CAR molecule specific for at least one target antigen.

In some specific embodiments, a CAR molecule further expressed by the genetically engineered cell/s used by the disclosed therapeutic methods, may comprise the following components: (i), at least one target-binding domain that specifically recognizes and binds a target antigen; (ii), at least one hinge and at least one transmembrane domain; and (iii), at least one intracellular T cell signal transduction domain.

Still further, the at least one target binding domain of the CAR molecule further expressed by the genetically engineered cell/s used by the disclosed therapeutic methods, may comprise at least one antibody or any antigen-binding fragment/s, portion/s or chimera/s thereof, specific for a target antigen.

In some embodiments, the antigen-binding fragment/s, portion/s or chimera/s of the antibody may comprise at least one of a single chain variable fragment (scFv), and/or nanobody.

In some embodiments, the target antigen targeted by the TCR and/or the CAR molecules further expressed by the genetically engineered cell/s used by the disclosed therapeutic methods, may be an antigen associated with a pathologic disorder.

In some embodiments, the genetically engineered cell/s used by the disclosed therapeutic methods, may further express a CAR molecule, comprising an anti-CD19 scFv.

In some embodiments, the target antigen targeted by the receptor molecule (e.g., CAR and/or TCR) further expressed by the genetically engineered cell/s used by the disclosed therapeutic methods may be at least one of: at least one TAA, at least one tumor specific antigen, at least one neoantigen, at least one viral antigen, at least one bacterial antigen, at least one fungal antigen and/or at least one parasite antigen.

In some embodiments, Neoantigens, a class of tumor-specific antigens, differ from the traditional tumor-associated antigen (TAA). TAA is not unique to tumor tissue as it is also present in normal tissues.

Still further, in some embodiments, the TAA may be a melanoma antigen.

In Some embodiments, the TAA recognized by the engineered cells used by the disclosed therapeutic methods, may be MART-1.

Still further, in some embodiments, at least one intracellular T cell signal transduction domain of the CAR molecule further expressed by the genetically engineered cell/s used by the disclosed therapeutic methods, may comprise at least one domain of tumor necrosis factor (TNF) receptor family member, and optionally, at least one domain of a T cell receptor (TCR) molecule.

In yet some further embodiments, the domain of the CAR molecule may be a costimulatory signaling domain selected from the group of 4-1BB, a cluster of differentiation 28 (CD28), and/or a cluster of differentiation 3 (CD3) zeta chain.

It should be understood that the disclosed method may use an of the engineered cells disclosed by the present disclosure, as defined herein before in connection with other aspects of the present disclosure. Still further, the disclosed therapeutic methods may use and administer ay of the nucleic acid sequences disclosed by the present disclosure and any cassette, construct, vector and/or vehicle and/or composition comprising the same.

In some embodiments, the disclosed therapeutic methods are appliable for an pathologic disorder. In some specific embodiments, such pathologic disorder may be at least one of a proliferative disorder, a metabolic condition, an inflammatory disorder, an infectious disease caused by a pathogen, an autoimmune-disease, and a cardiovascular disease.

In some embodiments, the method of the disclosure is directed at treating a pathologic disorder. In more specific embodiments, such disorder may be at least one of a proliferative disorder, an inflammatory disorder, an infectious disease caused by a pathogen, an autoimmune-disease as well as CVDs and metabolic conditions. Thus, in some specific embodiments, the subject treated by the method of the present disclosure may be a subject suffering of an immune-related disorder. An “Immune-related disorder” or “Immune-mediated disorder”, as used herein encompasses any condition that is associated with the immune system of a subject, more specifically through inhibition of the immune system, or that can be treated, prevented or ameliorated by reducing degradation of a certain component of the immune response in a subject, such as the adaptive or innate immune response. An immune-related disorder may include infectious condition (e.g., by a pathogen, specifically, viral, bacterial or fungal infections), inflammatory disease, autoimmune disorders, metabolic disorders and proliferative disorders, specifically, cancer. In some specific embodiments wherein the immune-related disorder or condition may be a primary or a secondary immunodeficiency.

In some specific embodiments, the methods of the disclosure may be used for treating proliferative disorders. As used herein to describe the present disclosure, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present disclosure may be applicable for treatment of a patient suffering from any one of non-solid and solid tumors. Malignancy, as contemplated in the present disclosure may be any one of melanomas, carcinomas, lymphomas, leukemias, myeloma and sarcomas. Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges. Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes. Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present disclosure can comprise but are not limited to hematological malignancies (including lymphoma, leukemia and myeloproliferative disorders), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. More particularly, the malignant disorder may be lymphoma. Non-limiting examples of cancers treatable according to the disclosure include hematopoietic malignancies such as all types of lymphomas, leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. Still further the disclosure relates to any neurological tumor, for example, neuroblastoma, astrocytoma, CNS lymphoma, neuroma, glioma, Chordoma, medulloblastoma, Oligodendroglioma, Craniopharyngioma, and any mixed neurological tumor.

It should be understood that the present disclosure thus encompasses the treatment of any of the malignancies described in this context, specifically any malignancies described in connection with associated TAAs as described herein before in connection with other aspects of the present disclosure.

In more specific embodiments, the therapeutic methods of the present disclosure may be applicable for treating at least one proliferative disorder. In more specific embodiments, the disorder may be at least one hematological malignancy, and/or at least one solid tumor.

In some specific embodiments, the disclosed methods may be applicable for treating hematological malignancies, such as acute lymphoblastic leukemia (ALL), diffuse large B-cell lymphoma (DLBCL), acute lymphoblastic leukemia (ALL), refractory Hodgkin's lymphoma, acute myeloid leukemia (AML), or multiple myeloma.

In yet some further embodiments, of the methods of the present disclosure may be also applicable for treating autoimmune disorders, that are also referred to as disorders of immune tolerance, when the immune system fails to properly distinguish between self and non-self-antigens.

Thus, according to some embodiments, the method of the present disclosure may be used for the treatment of a patient suffering from any autoimmune disorder. In some specific embodiments, the methods of the present disclosure may be used for treating an autoimmune disease such as for example, but not limited to, inflammatory bowel disease (IBD), ulcerative colitis, Crohn's disease, fatty liver disease, Lymphocytic colitis, Ischaemic colitis, Diversion colitis, Behget's syndrome, Indeterminate colitis, rheumatoid arthritis, systemic lupus erythematosus (SLE), Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

In some further embodiments, the therapeutic methods disclosed herein may be applicable for at least one metabolic disorder. In more specific embodiments, such metabolic disorder may comprise at least one cardiovascular disorder, liver diseases, diabetes and metabolic syndrome.

Metabolic disease is any of the diseases or disorders that disrupt normal metabolism, the process of converting food to energy on a cellular level. Thousands of enzymes participating in numerous interdependent metabolic pathways carry out this process. Metabolic diseases negatively affect the ability of the cell to perform critical biochemical reactions that involve the processing or transport of proteins (amino acids), carbohydrates (sugars and starches), or lipids (fatty acids). Numerous molecular pathways and thus, several organs, can be affected. Many of the metabolic diseases are caused by genetic mutations or by a combination of genetic and environmental factors. Some of the metabolic diseases or diseases associated with metabolic disorders include for example: diabetes (or diabetes mellitus), a group of common endocrine diseases characterized by sustained high blood sugar levels affecting nearly every major bodily organ; non-alcoholic steatohepatitis (NASH), a liver inflammation and damage caused by a buildup of fat in the liver; steatosis (or fatty change), an abnormal retention of fat (lipids) within a cell or organ, most often affects the liver, but can also occur in other organs, including the kidneys, heart, and muscle; obesity, a medical condition, in which excess body fat has accumulated to such an extent that it may negatively affect health; dyslipidemia, is the imbalance of lipids such as cholesterol, low-density lipoprotein cholesterol, (LDL-C), triglycerides, and high-density lipoprotein (HDL): cardiovascular diseases, a group of disorders of the heart and blood vessels and include coronary heart disease, cerebrovascular disease, rheumatic heart disease and other conditions.

In yet some other embodiments, the methods of the present disclosure may be also applicable for treating a subject suffering from an infectious disease. More specifically, such infectious disease may be any pathological disorder caused by a pathogen. As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts or a nematode.

In yet some further embodiments, the methods of the present disclosure may be applicable in boosting the immune response against a pathogen that may be in further specific embodiment, a viral pathogen or a virus. The term “virus” as used herein, refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. It should be noted that the term “viruses” is used in its broadest sense to include viruses of the families adenoviruses, papovaviruses, herpesviruses: simplex, varicella-zoster, Epstein-Barr (EBV), Cytomegalo virus (CMV), pox viruses: smallpox, vaccinia, hepatitis B (HBV), rhinoviruses, hepatitis A (HBA), poliovirus, respiratory syncytial virus (RSV), Middle East Respiratory Syndrome (MERS), Severe acute respiratory syndrome (SARS), rubella virus, hepatitis C (HBC), arboviruses, rabies virus, influenza viruses A and B, measles virus, mumps virus, human deficiency virus (HIV), HTLV I and II, Dengue virus and Zika virus.

In some further embodiments, the methods of the present disclosure may be applicable for immune-related disorder or condition that may be a pathologic condition caused by at least one pathogen. It should be appreciated that an infectious disease as used herein also encompasses any infectious disease caused by a pathogenic agent, specifically, a pathogen. Pathogenic agents include prokaryotic microorganisms, lower eukaryotic microorganisms, complex eukaryotic organisms, viruses, fungi, prions, parasites, yeasts, toxins and venoms. In yet some other specific embodiments, the methods and composition of the disclosure may be applicable for treating an infectious disease caused by bacterial pathogens. More specifically, a prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes. A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the disclosure. A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania. More specifically, in certain embodiments the methods and compositions of the disclosure may be suitable for treating disorders caused by fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous, and possess branched somatic structures (hyphae) surrounded by cell walls containing glucan or chitin or both, and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present disclosure also provides for the methods and compositions for the treatment of a pathological disorder caused by “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species. As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

In yet some further specific embodiments, the present disclosure provides compositions and methods for use in the treatment, prevention, amelioration or delay the onset of a pathological disorder, wherein said pathological disorder is a result of a prion. As used herein, the term “prion” refers to an infectious agent composed of protein in a misfolded form. Prions are responsible for the transmissible spongiform encephalopathies in a variety of mammals, including bovine spongiform encephalopathy (BSE, also known as “mad cow disease”) in cattle and Creutzfeldt-Jakob disease (CJD) in humans. All known prion diseases affect the structure of the brain or other neural tissue and all are currently untreatable and universally fatal. It should be appreciated that an infectious disease as used herein also encompasses any pathologic condition caused by toxins and venoms.

Thus, the methods of the present disclosure may offer a promising therapeutic modality for a variety of innate and acquired immunodeficiencies caused by immunosuppressive treatments (chemo- and radiotherapy), pathogenic infections, cancer and HSCT. More specifically, Immunodeficiency (or immune deficiency) is a state in which the immune system's ability to fight infectious disease and cancer is compromised or entirely absent. Most cases of immunodeficiency are acquired (“secondary”) due to extrinsic factors that affect the patient's immune system. Examples of these extrinsic factors include viral infection, specifically, HIV, extremes of age, and environmental factors, such as nutrition. In the clinical setting, the immunosuppression by some drugs, such as steroids, can be either an adverse effect or the intended purpose of the treatment. Examples of such use are in organ transplant surgery as an anti-rejection measure and in patients suffering from an overactive immune system, as in autoimmune diseases. Immunodeficiency also decreases cancer immuno-surveillance, in which the immune system scans the cells and kills neoplastic ones. Still further, Primary immunodeficiencies (PID), also termed innate immunodeficiencies, are disorders in which part of the organism immune system is missing or does not function normally. To be considered a primary immunodeficiency, the cause of the immune deficiency must not be caused by other disease, drug treatment, or environmental exposure to toxins). Most primary immune-deficiencies are genetic disorders; the majority is diagnosed in children under the age of one, although milder forms may not be recognized until adulthood. While there are over 100 recognized PIDs, most are very rare. There are several types of immunodeficiency that include, Humoral immune deficiency (including B cell deficiency or dysfunction), which generally includes symptoms of hypogammaglobulinemia (decrease of one or more types of antibodies) with presentations including repeated mild respiratory infections, and/or agammaglobulinemia (lack of all or most antibody production) and results in frequent severe infections (mostly fatal); T cell deficiency, often causes secondary disorders such as acquired immune deficiency syndrome (AIDS); Granulocyte deficiency, including decreased numbers of granulocytes (called as granulocytopenia or, if absent, agranulocytosis) such as of neutrophil granulocytes (termed neutropenia); granulocyte deficiencies also include decreased function of individual granulocytes, such as in chronic granulomatous disease; Asplenia, where there is no function of the spleen; and Complement deficiency in which the function of the complement system is deficient. Secondary immunodeficiencies occur when the immune system is compromised due to environmental factors. Such factors include but are not limited to radiotherapy as well as chemotherapy. While often used as fundamental anti-cancer treatments, these modalities are known to suppress immune function, leaving patients with an increased risk of infection; indeed, infections were found to be a leading cause of patient death during cancer treatment. Neutropenia was specifically associated with vulnerability to life-threatening infections following chemotherapy and radiotherapy. In more specific embodiments, such secondary immunodeficiency may be caused by at least one of chemotherapy, radiotherapy, biological therapy, bone marrow transplantation, gene therapy, adoptive cell transfer or any combinations thereof.

Still further, the genetically engineered cell/s of the present disclosure exogenously express at least one protein involved directly or indirectly in at least one metabolic pathway. The expression of such protein by the at least one genetically engineered cell of the T lineage in the subject, results in various effects displayed by the engineered cells used (I), and/or by the treated subject (II). Thus, in some embodiments, the expression of such protein by the at least one genetically engineered cell of the T lineage in the subject, results in at least one of:

In some embodiments (I), at least one of: (i) increased cytokine secretion; (ii) increased expression of activation markers; (iii) increased glycolysis; (iv) improvement in metabolic parameters; and (v) reduced expression of exhaustion markers; (vi) increased proliferation; (vii) increased cell survival; and/or (viii) increase in oxidative phosphorylation; by the genetically engineered cell of the T lineage; and/or In some further embodiments (II), at least one of: (i) increased survival; (ii) reduced relapse rate; and/or (iii) a long-term effect; in said subject.

It should be understood that in some embodiments, the indicated results of the expression of the protein by the disclosed genetically engineered cell may be in response to a specific stimulation in an in vivo and/or in vitro/ex vivo setting, for example, activation of T-cells (e.g., by anti-CD3 antibodies and the like). In yet some further embodiments, the improvement in metabolic parameters comprises at least one of: increased ATP content, glucose uptake and/or mitochondrial mass.

In some embodiments, the at least one genetically engineered cell of the T cell lineage or a cell population comprising at least one of the cell used by the disclosed therapeutic methods, may be of an autologous or allogeneic source.

As indicated herein, the present disclosure provides method allowing in vivo as well as ex-vivo or in vitro genetic engineering of cells of the T lineage to express the molecules of the present disclosure. In case the engineering of the cells is performed ex vivo or in vitro, the engineered cells are transferred back to the subject, by adoptive transfer. In such case, the method comprising the step of administering to the subject (a), the engineered cells or any compositions thereof.

The term “adoptive transfer” as herein defined applies to all the therapies that consist of the transfer of components of the immune system, specifically cells that are already capable of mounting a specific immune response. In such option, the insertion of the nucleic acid sequence encoding the at least one molecule involved directly or indirectly in at least one metabolic pathway and optionally, the at least one receptor disclosed herein, is performed in cells of an autologous or allogeneic source, that are then administered to the subject, specifically, by adoptive transfer.

In some embodiments, the cells that express, comprise, transduced or transfected with the nucleic acid molecule/s of the present disclosure, that encodes at least one molecule involved directly or indirectly in at least one metabolic pathway, may be cells of an autologous source. The term “autologous” when relating to the source of cells, refers to cells derived or transferred from the same subject that is to be treated by the methods of the invention. The term “allogenic” when relating to the source of cells, refers to cells derived or transferred from a different subject, referred to herein as a donor, of the same species.

In some alternative or additional embodiments, the cells of the T lineage in accordance with the present disclosure may be engineered in vivo. According to such embodiments, the treated subject is treated with at least one nucleic acid sequence encoding the at least one molecule involved directly or indirectly in at least one metabolic pathway, or with any cassette, vector, gene editing system or vehicle comprising the same.

The disclosure provides nucleic acid molecules, sequences encoding the encoding the at least one molecule involved directly or indirectly in at least one metabolic pathway and the engineered CAR T of the present disclosure, cassette, and methods, cells, uses and compositions thereof. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art. Still further, it should be understood that the disclosure encompasses as additional aspects thereof any vector or vehicle that comprise any of the nucleic acid molecule/s of the disclosure or any cassettes described by the disclosure.

Still further, in some embodiments, the nucleic acid molecule/s of the disclosure or any cassette used by the disclosure, specifically, cassettes comprising nucleic acid sequences encoding least one molecule involved directly or indirectly in at least one metabolic pathway, may be comprised within a nucleic acid vector. In more specific embodiments, such vector may be any one of a viral vector, a non-viral vector and a naked DNA vector.

Vectors, as used herein, are nucleic acid molecules of particular sequence can be incorporated into a vehicle that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g., plasmids, cosmids, minicircles, phage, viruses, etc., useful for transferring nucleic acids into target cells may be applicable in the present disclosure. The vectors comprising the nucleic acid(s) may be maintained episomally, e.g., as plasmids, minicircle DNAs, viruses such cytomegalovirus, adenovirus, etc., or they may be integrated into the target cell genome, through homologous recombination or random integration, e.g., retrovirus-derived vectors such as AAV, MMLV, HIV-1, ALV, etc. Vectors may be provided directly to the subject cells. In other words, the cells are contacted with vectors comprising the nucleic acid molecules, and/or cassettes of the disclosure that comprise the nucleic acid sequence encoding the encoding the immune effector of interest and the engineered CAR T disclosed herein such that the vectors are taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV). More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector. As indicated above, in some embodiments, viral vectors may be applicable in the present disclosure. The term “viral vector” refers to a replication competent or replication-deficient viral particle which are capable of transferring nucleic acid molecules into a host. The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present disclosure include baculoviridiae, parvoviridiae, picornoviridiae, herepesviridiae, poxviridiae, adenoviridiae, picotmaviridiae. The term recombinant virus includes chimeric (or even multimeric) viruses, i.e., vectors constructed using complementary coding sequences from more than one viral subtype.

In some embodiments, the nucleic acid molecules, and/or cassette of the disclosure may be comprised within a retroviral vector. A retroviral vector, as used herein consists of proviral sequences that can accommodate the nucleic acid molecule encoding the engineered CAR T disclosed herein, to allow incorporation of both into the target cells. The vector may also contain viral and cellular gene promoters, to enhance expression of the nucleic acid molecule encoding the encoding the immune effector of interest and the engineered CAR T disclosed herein in the target cells. Retroviral vectors stably integrate into the dividing target cell genome so that the introduced gene is passed on and expressed in all daughter cells. They contain a reverse transcriptase that allows integration into the host genome.

In some alternative embodiments, the nucleic acid molecules, and/or cassette of the present disclosure may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express nucleic acid sequences encoding the encoding the immune effector of interest and the engineered CAR T disclosed herein for months in quiescent cells. Production systems for rAAV vectors typically consist of a DNA-based vector containing a transgene expression cassette, which is flanked by inverted terminal repeats. Construct sizes are limited to approximately 4.7-5.0 kb, which corresponds to the length of the wild-type AAV genome. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of nucleic acid molecules encoding the encoding the immune effector of interest and the engineered CAR T disclosed herein when rAAV is used as gene delivery tool. Indeed, significant differences in transduction efficacy of various serotypes for particular tissues and cell types have been described.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB but leads to significantly shortened onsets of expression in comparison with conventional single-stranded AAV expression constructs (ssAAV). Thus, in some embodiments, ssAAV may be applicable as a viral vector by the methods of the disclosure.

In yet some further embodiments, HDAd vectors may be suitable for the at least one molecule involved directly or indirectly in at least one metabolic pathway, encoding sequences, cells, compositions and methods of the present disclosure. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long-term expression of the transgene. Still further, in some embodiments, SV40 may be used as a suitable vector by the methods of the disclosure. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10(12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome. In yet some alternative embodiments, lentiviral vectors may be used in the present disclosure. Lentiviral vectors are derived from lentiviruses which are a subclass of Retroviruses. Commonly used retroviral vectors are “defective”, i.e., unable to produce viral proteins required for productive infection. Rather, replication of the vector requires growth in a packaging cell line. To generate viral particles comprising the nucleic acid molecules, vectors and/or cassette in accordance with the disclosure, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid molecules, vectors and/or cassette of the disclosure that contains the nucleic acids sequence encoding the CAT-T of the disclosure, into packaging cell lines and of collecting the viral particles that are generated by the packaging lines are well known in the art. Nonviral vectors, in accordance with the disclosure, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA entrance into the targeted cells is facilitated.

In more specific embodiments, the vector may be a naked DNA vector. More specifically, such vector may be for example, a plasmid, minicircle or linear DNA. Naked DNA alone may facilitate transfer of a gene (2-19 kb) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria. Linear DNA or Doggybone™ are double-stranded, linear DNA construct that solely encodes an antigen expression cassette, comprising antigen, promoter, polyA tail and telomeric ends. It should be appreciated that all DNA vectors disclosed herein, may be also applicable for all nucleic acid molecules, vectors and/or cassettes used in the methods and compositions of the disclosure, as described herein. Still further, it must be appreciated that the present disclosure further provides any vectors or vehicles that comprise any of the nucleic acid molecules, vectors and/or nucleic acid cassettes disclosed by the present disclosure, as well as any host cell expressing the nucleic acid molecules, and/or nucleic acid cassettes disclosed herein.

As discussed herein, the present disclosure further encompasses any gene editing component/s and/or systems that enables and facilitates the insertion of the nucleic acid sequence that encodes any protein involved directly or indirectly with exogeneous expression of molecules involved with a metabolic pathway, into a target site within the genome of any target cell.

In some embodiments, a gene editing component of the gene editing system disclosed herein, may be any one of a site-specific nuclease, a class switch recombination, a site specific integrase and a site-specific recombinase.

In some specific embodiments, a gene editing component useful in the systems of the present disclosure may be the CRISPR/Cas.

More specifically, in some embodiments, the HK, GLUT3, PFK-1 and/or PKM encoding nucleic acid sequences (e.g., in a nucleic acid cassette) is inserted into the appropriate target genomic locus within a cell of the T lineage using a site-specific nuclease. The nuclease may be one of the following: CRISPR/Cas9/Cpf1/CTc(1/2/3), SpCas9, SaCas9, engineered CAS9, ZFN, TALEN, Homing endonuclease, Meganuclease, Mega-TALEN. The nuclease may be coded on a DNA vector such as a plasmid, a mini-circle or a viral vector. Alternatively, the mRNA coding for the nuclease may be delivered, or the nuclease may be delivered as a protein. A guide RNA may be provided or a DNA vector coding for a guide RNA. Integration catalyzed by a nuclease may utilize homologous arms flanking the DNA to be inserted or utilize recognition sites for the site-specific nuclease when such were coded preceding and or following the DNA to be inserted. Delivery of the nuclease or the vector coding for the nuclease can take place in vivo or ex vivo using autologous or allogeneic cells, as will be discussed herein after.

A further aspect of the present disclosure relates to a therapeutically effective amount of at least one of: (a), at least one genetically engineered hematopoietic cell/s or a cell population comprising at least one of the genetically engineered cell/s. It should be noted that the genetically engineered cell comprises and/or expresses (e.g., exogenously) at least one nucleic acid sequence encoding at least one molecule involved directly or indirectly in at least one metabolic pathway. Still further, in some embodiments of the disclosed use, the cell/s optionally further expresses at least one receptor molecule; (b), at least one nucleic acid sequence encoding the at least one molecule involved directly or indirectly in at least one metabolic pathway, or any cassette, vector and/or gene editing system comprising the nucleic acid sequence; and/or (c), a composition comprising said genetically engineered cell/s of (a) and/or the nucleic acid sequence of (b), for use in a method for treating, preventing, ameliorating, inhibiting or delaying the onset of a pathologic disorder in a mammalian subject. In some embodiments, the genetically engineered hematopoietic cells may be any lymphocyte cells. In yet some further embodiments, the genetically engineered lymphocytes of the present disclosure may be genetically engineered cells of the T cell lineage.

In some embodiments of the disclosed effective amount for use, the genetically engineered cell of the T cell lineage or a cell population comprising at least one of the cell/s used herein, may be any of the cells as defined by the present disclosure, and/or any of the compositions as defined by the present disclosure.

The term “effective amount” relates to the amount of an active agent present in a composition, specifically, the genetically engineered cell of the disclosure as described herein or composition comprising same that is needed to provide a desired level of active agent in the bloodstream or at the site of action in an individual (e.g., the thymus or bone marrow) to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of the genetically engineered cell of the disclosure can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

As described herein above, the disclosure provides in some aspects thereof therapeutic and prophylactic methods. It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, a pathologic condition and illness, symptoms or undesired side effects. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. The term “amelioration” as referred to herein, relates to a decrease in the symptoms, and improvement in a subject's condition brought about by the cells, compositions and methods according to the disclosure, wherein said improvement may be manifested in the forms of inhibition of pathologic processes associated with the disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with. The term “eliminate” relates to the substantial eradication or removal of the pathologic symptoms and possibly pathologic etiology, optionally, according to the methods of the disclosure described herein. The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the disclosure.

As indicated above, the methods and compositions provided by the present disclosure may be used for the treatment of a “pathological disorder”, specifically, immune-related disorders as specified by the disclosure, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein. It should be appreciated that any of the methods and compositions described by the disclosure may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms. The present disclosure relates to the treatment of subjects or patients, in need thereof. By “patient” or “subject in need” it is meant any organism who may be affected by the above-mentioned conditions, and to whom the therapeutic and prophylactic methods herein described are desired, including any vertebrate, specifically mammals such as humans, domestic and non-domestic mammals such as canine and feline subjects, bovine, simian, equine and rodents, specifically, murine subjects. More specifically, the methods of the disclosure are intended for mammals. By “mammalian subject” is meant any mammal for which the proposed therapy is desired, including human, livestock, equine, canine, and feline subjects, most specifically humans.

A further aspect provided by the present disclosure relates to a method for improving activity and/or survival of at least one hematopoietic cell. The method disclosed herein may comprise the step of contacting at least one cell with an effective amount of at least one nucleic acid sequence encoding at least one molecule involved directly or indirectly in at least one metabolic pathway, or any cassette, vector and/or gene editing system comprising the nucleic acid sequence, the cell optionally further expresses at least one receptor molecule. In some embodiments, the genetically engineered hematopoietic cells may be any lymphocyte cells. In yet some further embodiments, the genetically engineered lymphocytes of the present disclosure may be genetically engineered cells of the T cell lineage.

In some embodiments, the protein encoded by the at least one nucleic acid molecule used by the disclosed methods, may be a protein involved directly or indirectly in the at least one metabolic pathway by the at least one cell of the T lineage results in at least one of: (i) increased cytokine secretion; (ii) increased expression of activation markers; (iii) increased glycolysis; (iv) improvement in metabolic parameters; (v) increased cell survival; (vi) increased cytotoxicity; (vii) reduced expression of exhaustion markers; and/or (viii) increased oxidative phosphorylation, by the cell.

In some embodiments, the step of contacting the cell of the T lineage with the at least one nucleic acid sequence, is performed by the disclosed methods in vivo, in vitro or ex vivo.

In yet some further embodiments, the step of contacting the cell of T lineage with the at least one nucleic acid sequence, may be performed by the disclosed methods in vivo in a subject suffering from at least one pathologic disorder. In more specific embodiments, the contacting step may comprise administering to the subject an effective amount of the nucleic acid sequence encoding at least one molecule involved directly or indirectly in at least one metabolic pathway, any cassette, vector and/or gene editing system comprising the nucleic acid sequence or any composition thereof.

In some embodiments, the cell of the disclosed methods, may be a cell of a subject suffering from at least one of a proliferative disorder, a metabolic condition, an inflammatory disorder, an infectious disease caused by a pathogen, an autoimmune-disease, and a cardiovascular disease.

Still further, in some embodiments, the contacting step of the cell of T lineage with the at least one nucleic acid sequence, may be performed by the disclosed methods in vitro or ex vivo, thereby obtaining genetically engineered cell/s of the T lineage, or a cell population comprising at least one of the cell/s.

In some embodiments, the cells may be of autologous or allogeneic source.

In yet some further embodiments of the disclosed methods, the cells produced are as defined by the present disclosure.

A further aspect of the present disclosure relates to a therapeutically effective amount of at least one of: (a), at least one genetically engineered hematopoietic cell/s or a cell population comprising at least one of the genetically engineered cell/s. The genetically engineered cell used herein, comprise and/or expresses at least one nucleic acid sequence encoding at least one molecule involved directly or indirectly in at least one metabolic pathway. In some embodiments the nucleic acid sequence may be exogenous nucleic acid sequence. These cells may optionally further express at least one receptor molecule; (b), at least one nucleic acid sequence encoding the at least one molecule involved directly or indirectly in at least one metabolic pathway, or any cassette, vector and/or gene editing system comprising said nucleic acid sequence; and (c), at least one composition comprising the genetically engineered cell/s of (a) and/or the nucleic acid sequence of (b), for use in a method for improving activity and/or survival of at least one cell of the T-lineage. In some embodiments, the genetically engineered hematopoietic cells may be any lymphocyte cells. In yet some further embodiments, the genetically engineered lymphocytes of the present disclosure may be genetically engineered cells of the T cell lineage.

In some embodiments, the genetically engineered cell of the T cell lineage or a cell population of the disclosed use, may comprise at least one of the cell/s as defined by the present disclosure, and the composition used herein, may be an of the compositions as defined by the present disclosure.

In some embodiments, the step of contacting the hematopoietic cell with the at least one nucleic acid sequence, by the disclosed use, may be performed in vivo in a subject suffering from at least one pathologic disorder. According to such embodiments, the contacting step may comprise administering to the subject an effective amount of the nucleic acid sequence encoding at least one molecule involved directly or indirectly in at least one metabolic pathway, any cassette, vector and/or gene editing system comprising the nucleic acid sequence or any composition thereof.

In some embodiments if the disclosed effective amount for use, the at least one pathologic disorder may be at least one of a proliferative disorder, a metabolic condition, an inflammatory disorder, an infectious disease caused by a pathogen, an autoimmune-disease, and a cardiovascular disease.

Still further, in some embodiments, the contacting step of the cell/s of T lineage with the at least one nucleic acid sequence may be performed in vitro or ex vivo, thereby obtaining genetically engineered cell/s of the T lineage, or a cell population comprising at least one of the cell/s.

In some further embodiments, the cells are of autologous or allogeneic source.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Thus, as used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, and medical arts.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub combination or as suitable in any other described embodiment of the present disclosure. Certain features described in the context of various embodiments are not to be considered essential features of those embodiments, unless the embodiment is inoperative without those elements.

Various embodiments and aspects of the present disclosure as delineated hereinabove and as claimed in the claims section below find experimental support in the following examples.

Disclosed and described, it is to be understood that this disclosure is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present disclosure will be limited only by the appended claims and equivalents thereof.

It must be noted that, as used in this specification and the appended claims, the singular forms “a”, “an” and “the” include plural referents unless the content clearly dictates otherwise.

### EXAMPLES

Without further elaboration, it is believed that one skilled in the art can, using the preceding description, utilize the present disclosure to its fullest extent. The following preferred specific embodiments are, therefore, to be construed as merely illustrative, and not limitative of the claimed disclosure in any way.

### Experimental Procedures

### Patient PBMCs and Cell Lines

All of the PBMCs used in this study were from healthy donors obtained from the Israeli Blood Bank (Sheba Medical Center, Tel-Hashomer, Israel). Melanoma cell lines HLA-A2⁺/MART-1⁺ (624.38) and HLA-A2⁻/MART-1⁺ (888) were generated at the Surgery Branch (National Cancer Institute, National Institutes of Health, Bethesda, MD)[Hoogi et al., J Immunother Cancer. 2019 Sep. 9; 7(1):243]. 888A2 is an HLA-A2-transduced line derived from 888. SK-MEL23 is an HLA-A2⁺ melanoma cell line (CVCL_6027). A375 (CVCL_0132) melanoma is HLA-A2⁺/MART-1⁻. Adherent cells were cultured in DMEM (Invitrogen, Carlsbad, CA), supplemented with 10% heat-inactivated Fetal Bovine Serum (Biological Industries, Beth Haemek, Israel) and were maintained in a 37° C. and 5% CO₂ incubator. CD19-expressing B-cell targets were K562 (CCL_243; which is CD19 negative) and K562-CD19. K562-CD19 was engineered to express the CD19 antigen following retroviral transduction with a CD19 encoding vector. Non-adherent tumor cells were cultured in RPMI (Invitrogen, Carlsbad, CA), supplemented with 10% heat-inactivated Fetal Bovine Serum (Biological Industries, Beth Haemek, Israel) and were maintained in a 37° C. and 5% CO₂ incubator. Lymphocytes were cultured in BioTarget medium (Biological Industries, Beth Haemek, Israel) supplemented with 10% heat-inactivated FBS and 300 IU/ml IL-2 (Peprotech, Israel) and maintained at 37° C. and 5% CO₂.

### TCR and Retroviral Constructs

The α and β chains from the previously characterized TCRs specific for MART-1₂₆₋₃₅, as denoted by SEQ ID NO: 9, termed F4 (or DMF4) and F5 (or DMF5) were subcloned into the MSGV1 vector as described previously (Haga-Friedman A. et al., J Immunol 2012 Jun. 1; 188(11):5538-46. doi: 10.4049/jimmunol.1103020. Epub 2012 Apr. 27). The retroviral vector backbone used in this study, pMSGV-1, is a derivative of the MSCV-based splice-gag vector which uses a murine stem cell virus (MSCV) long terminal repeat and has been previously described.

### Transduction of PBLs

For transient virus production, transfection of 2.5×10⁵ 293GP cells with 2 μg DNA of MSGV1-based retroviral construct and 1 μg envelop plasmid (VSV-G) was performed using JetPrime transfection reagent (Polyplus, France). After 4 h, the medium was replaced. Retroviral supernatant was collected 48 h after the DNA transfection. Freshly isolated PBLs were stimulated in the presence of 50 ng/ml OKT3 (eBioscience, San Diego, CA). 2 days after stimulation, lymphocytes were transduced consecutively, first with a TCR or CAR, and 24 h after this, with supernatant encoding the CSR or control. Transduction was performed in non-treated tissue culture dishes (Nunc, Rochester NY) that had been pre-coated with RetroNectin (Takara, Japan) and retroviral vectors as previously described.

### Gene-Expression Analysis by Real-Time PCR

cDNA (cDNA) was synthesized using iScript cDNA Synthesis kit from BIORAD Reverse transcriptases (RTs) following RNA isolation with Total RNA Mini Kit from GenAid industry. Gene-Expression Analysis by qRT-PCR was used to quantify expression levels of certain candidate genes. real-time PCR was performed in triplicates with SYBR GreenER qPCR. Two microliters of a ten-fold dilution of the cDNA were added to the qPCR reaction mixture [10 μl SYBR GreenER qPCR SuperMix for ABI PRISM (2×), 1 μl forward primer (10 μM), 1 μl reverse primer (10 M), 6 l double-distilled water]. The cycle conditions of qPCR were 95° C. 10 min, followed by 40 cycles of 95° C. 15 sec and 60° C. for 1 min.

RNA levels of each candidate gene as quantified by the PCR system were normalized to a housekeeping gene, L13a or B-actin.

### FACS Analysis and Antibodies

Fluorophore-labeled anti-human NGFR, CD8, CD4, CD137, TIM3, LAG3, CD69, CD25 CCR7 CD45RO were purchased from Biolegend (San Diego, CA, USA). Immunofluorescence, analyzed as the relative log fluorescence of live cells, was measured using a CyAn-ADP flow cytometer (Beckman Coulter, Brea, CA, USA). The fluorochromes conjugated to antibodies used: PE (FL-2), FITC (FL-1), PE-Cye5 (FL-4) and APC (FL-8) Approximately 1×10⁴-1×10⁵ cells were analyzed. Cells were stained in a FACS buffer made of PBS, 0.5% BSA, and 0.02% sodium azide. Antibodies used were PE-VO12 target TCR F4, tetramer target TCR F5, Protein L and PED streptavidin target CAR CD19.

### Intracellular Staining

Cells were fixed with pre-formaldehyde 4% and permeabilized using ice-cold 90% methanol for 20 min. Then, the cells were washed in FACS buffer, stained for anti-PFKM (Rabbit monoclonal [EPR10734(B)]abeam) and anti-HK2 antibody (Rabbit monoclonal [EPR20839]abeam) expression using a specific antibody and analyzed by flow cytometry, gated on the lymphocyte population.

### Cytokine Release Assays

Lymphocyte cultures were tested for reactivity in cytokine release assays using commercially available human ELISA kits for IL-2, IFN-γ, and TNF-α (R&D Systems, Minneapolis, MN, USA). For these assays, 1×10⁵ responder cells (T-cells) and 1×10⁵ stimulator cells (tumor cells) were incubated in a 200 μL culture volume in individual wells of 96-well plates. Stimulator cells and responder cells were co-cultured for 16 hours. Cytokine secretion was measured in culture supernatants diluted to be in the linear range of the assay.

### IncuCyte Killing Assay

Target cells had been previously transduced with mCherry. On the day of the experiment 1×10⁴ target cells were seeded on a flat 96 plate well. 1×10⁴ to 5×10⁴ T-cells were added to the wells, resulting in a ratio of 1:1 to 1:5 E:T. The plates were immediately placed in the IncuCyte and photographed every 3 hours for 24-72 h. The results were analyzed using the Incucyte program based on the orange integrated intensity of 3-4 replicates wells, normalized to the starting time.

### Established Tumor Assay

NSG mice were inoculated with 2 million tumor cells (either melanoma tumor SKMEL23/888A2) in 100 ul HBSS medium (Biological Industries, Beth Haemek, Israel) and 100 μl Cultrex matrix (Trevigen). It will be injected using an insulin syringe with a 27-gauge needle, in dorsal flank of 6-12-week NSG mice. Approximately 1 week upon tumor establishment (when tumor reaches approximately 7 mm), mice will be tail-injected with two intravenous injections of 5×10⁶ transduced lymphocytes resuspended in 200 μl HBSS medium were performed at day 7 and 14 after tumor inoculation, with MART1-specific TCR engineered-T cells (NGFR, PFK and PFK-I-GLUT3). Tumor growth was measured every 2-3 days in a blinded fashion using a caliper and calculated using the following formula: (Dxd²)×Π/6, where D is the largest tumor diameter and d its perpendicular one. All the procedures were performed according to the guidelines of the university committee for animal welfare.

### ATP Measurement Assay

The cellular ATP level was measured using ATP Determination Kit (Molecular Proves, Invitrogen) following the protocol by the manufacturer. Each sample was analyzed in triplicates in a 96 well flat bottom tissue culture plate, a standard reaction for ATP measurement was included in every set of experiment and bioluminescence was measured at 560 nm.

### Glucose Uptake Assay (2-NBDG)

T cells were starved with RPMI glucose free during 15 min and stained by 2-Deoxy-2-[(7-nitro-2,1,3-benzoxadiazol-4-yl) amino]-D-glucose, also known as 2-NBDG, is a fluorescent D-glucose analog. The consequent fluorescent activity is detected by flow cytometry (FITC).

Mitochondrial Mass staining

Mitochondrial staining was performed with Mitospy Green FM. After 30 mins incubation, immunofluorescence was measured as the relative log of fluorescence and the mean fluorescence intensity (MFI) of the gated cells using a CyAN-DP flow cytometer.

### Seahorse Assay

Extracellular acidification rate was measured from cells in non-buffered DMEM containing 5 mM glucose, 2 mM L-glutamine, and 1 mM sodium pyruvate, under basal conditions in response to glycolysis inhibitors: glucose 11.1 mM, 1.15 uM oligomycin and 100 mM 2-DG (All from sigma) on the XFe96 Extracellular Flux Analyzer (Agilent Technologies).

### Polar Metabolites Extraction for HPLC-MS

T cells were cultured with ¹³C6 labeled glucose added to RPMI without glucose during 30 min. Then, metabolites were extracted and analysed as described*. Shortly, T cells were washed twice with ice-cold PBS before LC-MS extraction solution (5:3:2 methanol:acetonitrile:H2O) was added at 0.3 mL per sample. The samples were shacked for 10 min at 4° C. and then, centrifuged at 16,000 g for 10 min at 4° C. Supernatants were transferred to glass HPLC vials and stored at −80° C. until analysis LC-MS analysis, as described by Mackay, G. M., Zheng, L., van den Broek, N. J. F. & Gottlieb, E. Analysis of cell metabolism using LC-MS and isotope tracers. Methods Enzymol. 561, 171-196 (2015).

### Statistical Analysis

A paired Student's log t-test was used to determine statistical significance. Data are reported as mean±SEM. Statistical values, including number of replicates (n), can be found in the figure legends. *p<0.05, **p<0.01, ***p<0.001, ****p<0.0001. For in vitro experiments, n=number of separate experiments. For in vivo work, n=number of individual animals.

### Example 1

### Establishment and Characterization of Metabolically Modified T-Cell

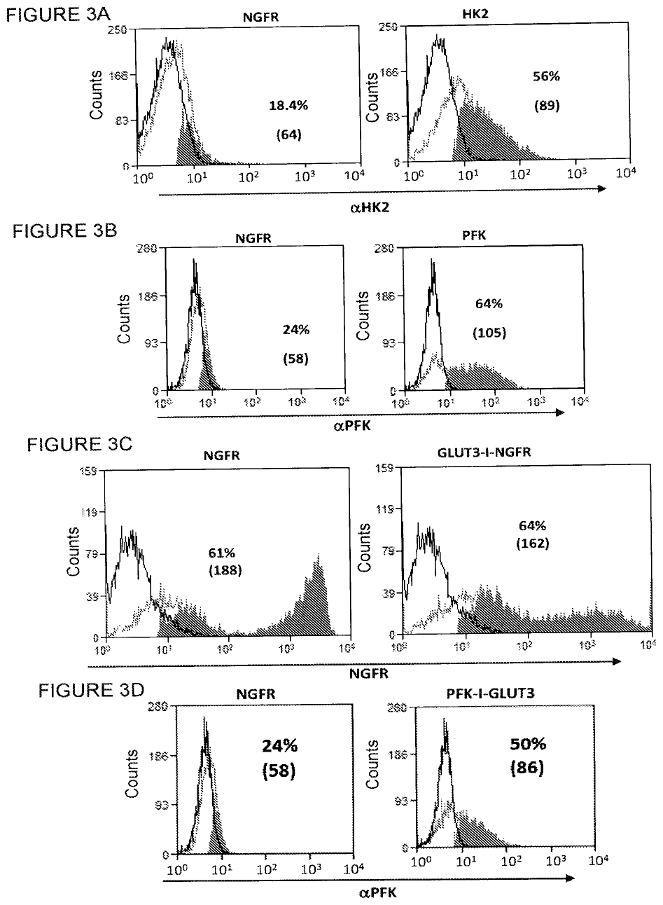

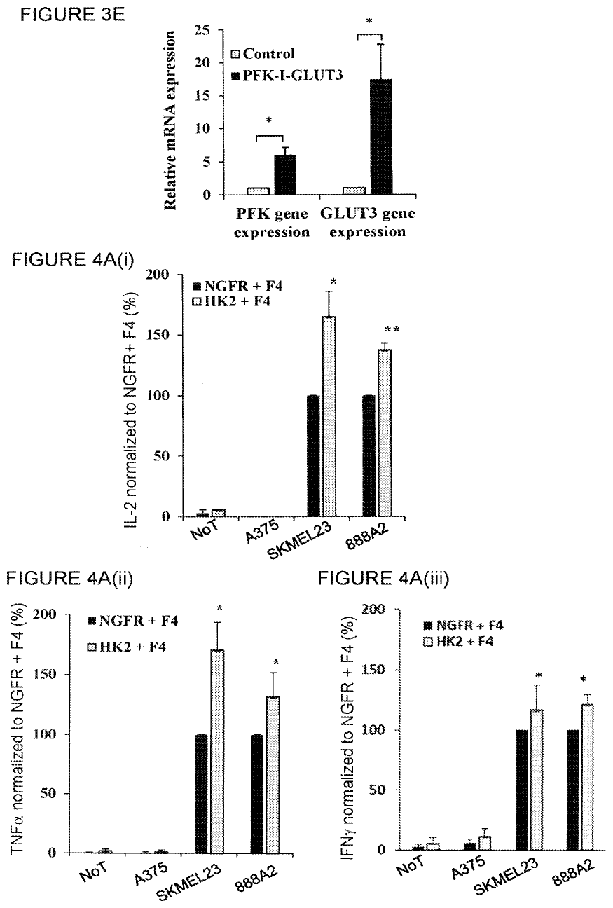

An effective metabolic capacity is required for a productive immune response. Accordingly, the inventors focused on modifying metabolic pathway, specifically the glycolysis pathway in T-cells, in order to enhance T-cell function and T-cell mediated immunity to tumors. To test this hypothesis, the inventors focused on a number of enzymes and a transporter in the glycolysis pathway, as illustrated by FIG. 1. First, the Inventors generated model system of T-cells which overexpress several main enzymes in the glycolysis pathway, i.e., Hexokinase-2 (HK2), Phosphofructokinase (PFK), pyruvate kinase (PKM), Glucose transporters (GLUT3), and combinations thereof. Initially, human PBLs were transduced with retroviral vectors encoding HK2 or PFK and the expression levels of the transduced genes were examined (FIG. 3A, 3B). Indeed, 56% of the cells infected with the vector encoding HK2 were HK2 positive compared to only 18.4% in control T-cells transduced with a truncated version of NGFR (a marker without endodomain) (FIG. 3A). Similarly, 64% of the cells infected with the vector encoding PFK were PFK positive compared to 24% in control NGFR cells (FIG. 3B).

The inventors further combined a truncated version of NGFR (reporter) with the GLUT3 gene and measured NGFR expression by FACS (FIG. 3C). Based on NGFR expression, 64% of the cells infected with the vector encoding GLUT3 and NGFR were positive (NGFR reporter for the GLUT3 expression) as compared to 61% NGFR control cells.

The inventors further created a plasmid, PFK-IRES-GLUT3, by combining the highly potent genes PFK and GLUT3, examining whether the combination achieve better results. The data gathered by the inventors indicate that the impact of transduction with glucose transporter is more significant early on after transduction (day 9-15), whereas the effect of PFK transduction was more sustained and noticeable from 2 weeks on, after transduction (day 18-30).

Indeed, the inventors were able to further express PFK enzyme and GLUT3 transporter in T cells. The percentage of PFK-I-GLUT3 positive cells was increased to 50% compared to 24% in control T-cells transduced with NGFR (FIG. 3D).

The relative expression of PFK and GLUT3 in T-cells transduced with PFK-I-GLUT3 were also shown at the mRNA level (FIG. 3E).

### Example 2

### Functional Assays (Cytokine Release and Activation Markers) of the Metabolically Modified T-Cell

During the cytotoxic anti-tumor immune response, T cells secrete several different cytokines once activated in the tumor microenvironment. The massive production of cytokines often supports the anti-tumor response as well as strengthens the proliferation/cytotoxicity of the T cells. The three main cytokines that provide such support include IFN-γ, TNF-α and IL-2 [Curtsinger J M. and Mescher M F. Curr Opin Immunol. 22(3):333-40 (2010); Sylvia Lee and Kim Margolin, Cancers (Basel). 3(4): 3856-3893 (2011); Showalter A et al. Cytokine. 97:123-132 (2017)]. Also, high levels of cytokine secretion often indicate a higher level of T cell activation [Aksoylar, H.-I., et al., Immunometabolism, 2(3), e200020 (2020)]. The inventors therefore tested whether the overexpression of the metabolic genes modify the extent of cytokine secretion in the culture.

To address this question and further examine T-cell function, the inventors used three T-cells model systems for activation:
a. Stimulation with OKT3: PBLs transduced with retroviral vectors encoding glycolysis related genes. The transduced cells were activated with T-cell specific monoclonal antibody OKT3;
b. F4/F5 TCR system: PBLs co-transduced with retroviral vectors encoding T-Cell Receptor (TCR) specific for Melanoma Antigen Recognized By T-Cells (MART-1) termed F4 or F5 and either the glycolysis related genes of the present disclosure or the control vector (NGFR/Mock). The transduced cells were activated with melanoma tumor cells expressing the respective antigen.
c. CD19 CAR system: PBLs co-transduced with constructs for chimeric antigen receptor (CAR) consisting of an anti-CD19 scFv (single chain variable fragment) coupled to the costimulatory signaling domain CD28 or 4-1BB and retroviral vectors encoding either the glycolysis related genes or the control vector (NGFR/Mock). The transduced cells were activated with bone-derived lymphoblast expressing CD19.

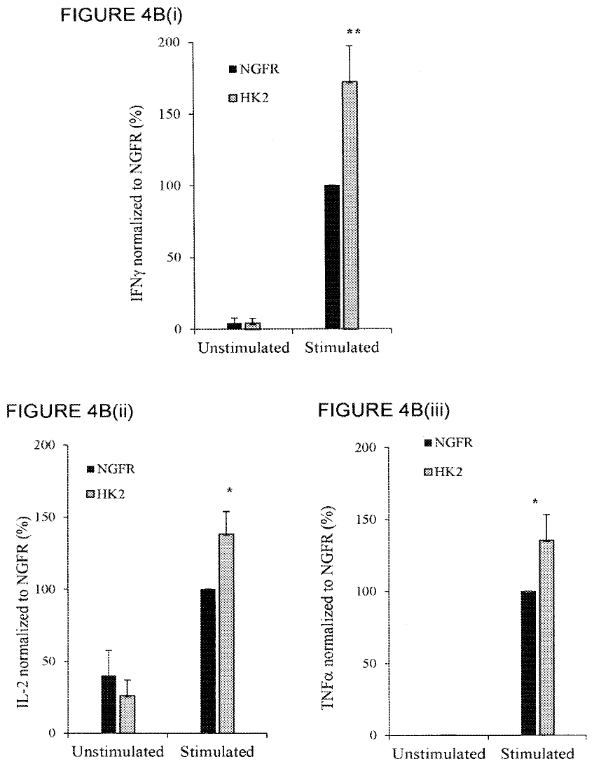

Based on these systems, the effect of expressing glycolysis related genes on cytokine secretion, was first evaluated (FIGS. 4 to 7). Indeed, in F4 TCR system, HK2 transduced cells showed an increase of 65% and 37% in IL-2 secretion when incubated with positive melanoma cell lines (SKMEL23 and 888A2) compared to control NGFR transduced cells (FIG. 4A). Similarly, an increase of 71%, 38% and 35% in IFNγ, IL2 and TNFα secretion was observed in HK2 transduced cells following stimulation with OKT3 compared to control NGFR cells (FIG. 4B).

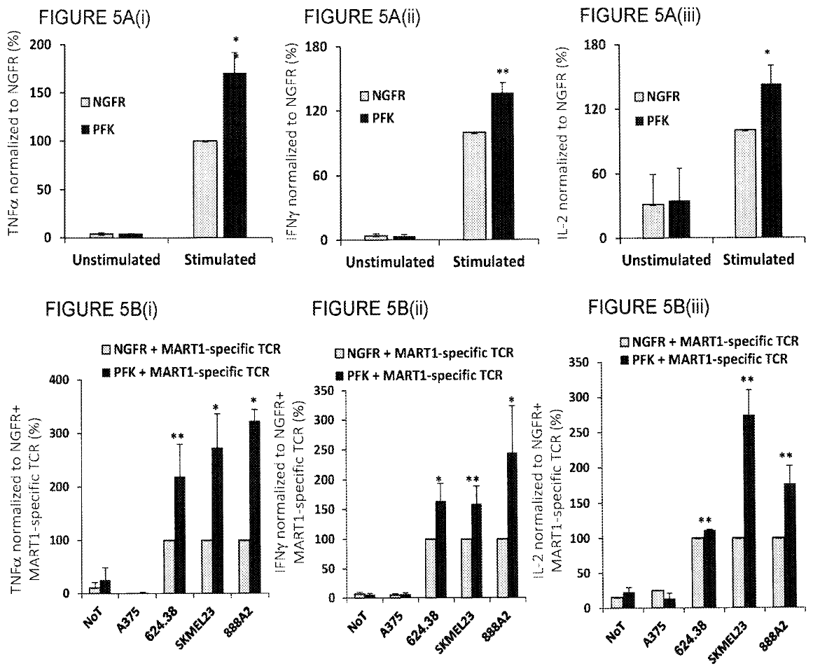

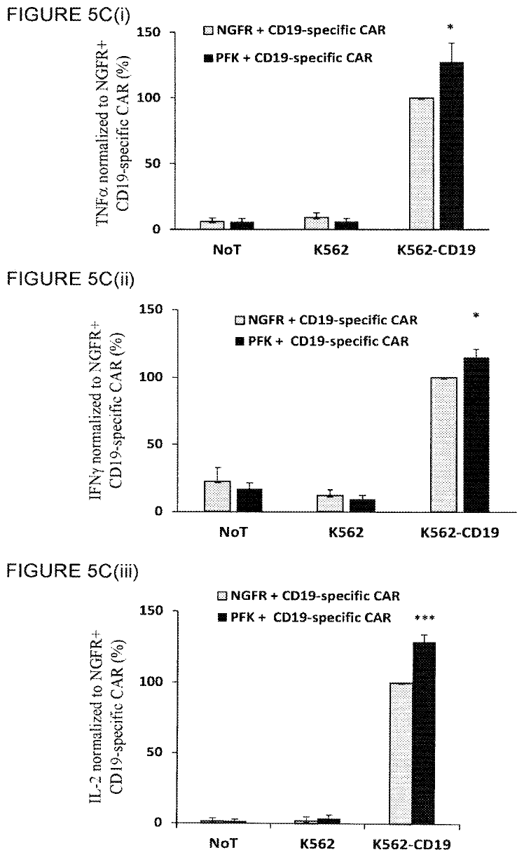

Similarly, an improvement of 70%, 43% and 36% in TNFα, IL2 and IFNγ secretion was observed in PFK-NGFR transduced cells following stimulation with OKT3 compared to control NGFR cells (FIG. 5A). In the F4 TCR system, a significant increase in cytokine secretion (ranging from 11% to 223%) by the PFK expressing cells was also observed (FIG. 5B). Moreover, in the CD19 CAR system, PFK transduced cells showed an increase of 29% in IL-2 secretion when incubated with K562-CD19 cell line compared to control NGFR transduced cells (FIG. 5C).

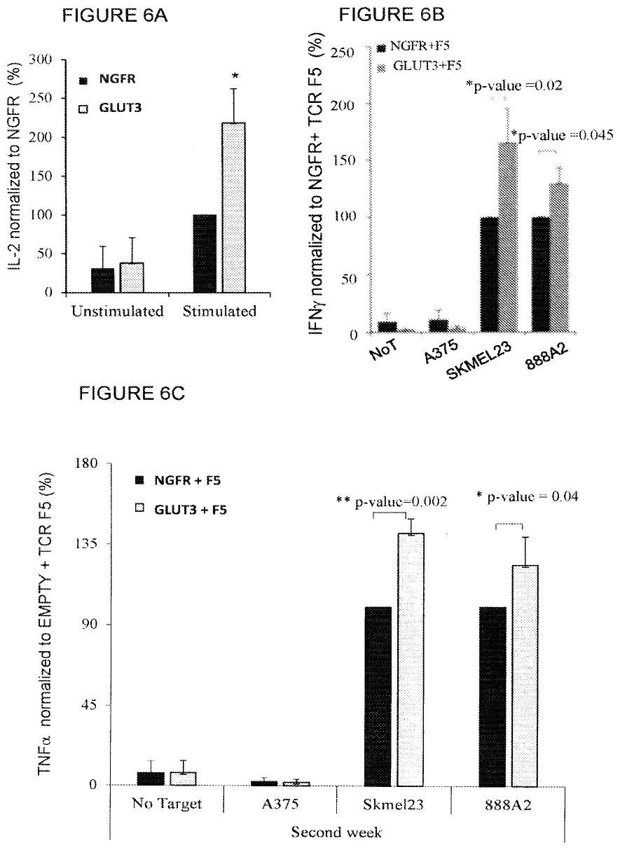

An improvement of 118% in IL-2 secretion after stimulation with OKT3 was also observed in GLUT3 transduced cells compared to control NGFR (FIG. 6A). Furthermore, an improvement of 30 and 66% in IFNγ secretion was observed in culture medium of F5 TCR GLUT3 transduced cells when incubated with positive melanoma cell lines (888A2 and SKMEL23 respectively) compared to control F5 NGFR transduced cells (FIG. 6B).

Similarly, a significant increase in TNFα, IL2 and IFNγ secretion was observed in culture medium of MART1 specific TCR GLUT3 transduced cells when incubated with positive melanoma cell lines (624.38, 888A2 and SKMEL23) compared to control cells expressing only the TCR and a control gene NGFR transduced cells (FIG. 6C).

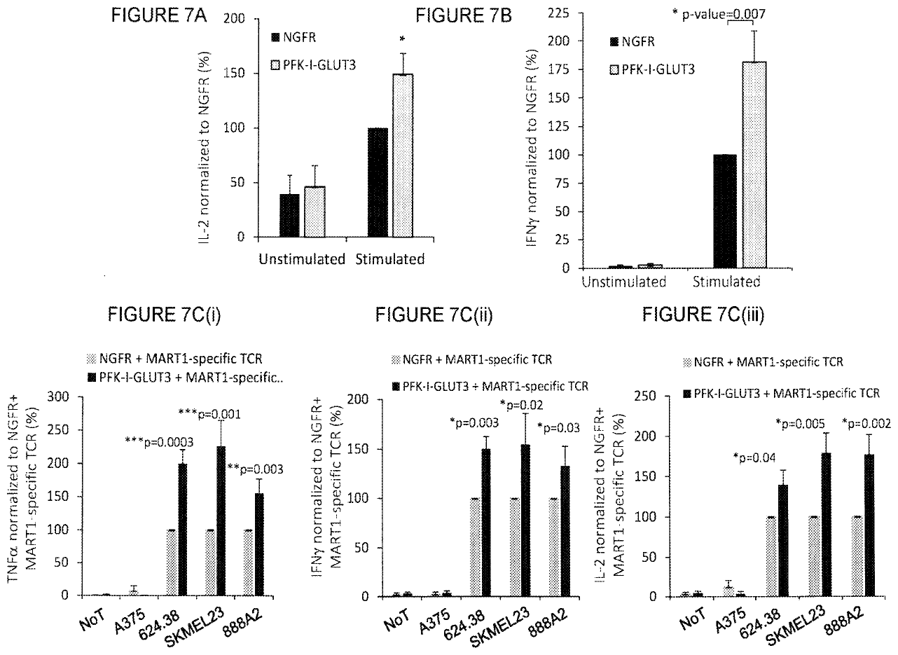

Thus, the combination of PFK and GLUT3 yielded a substantial increase in cytokine secretion in either the OKT-3 induced system (FIG. 7A-7B) or MART1 TCR system (FIG. 7C).

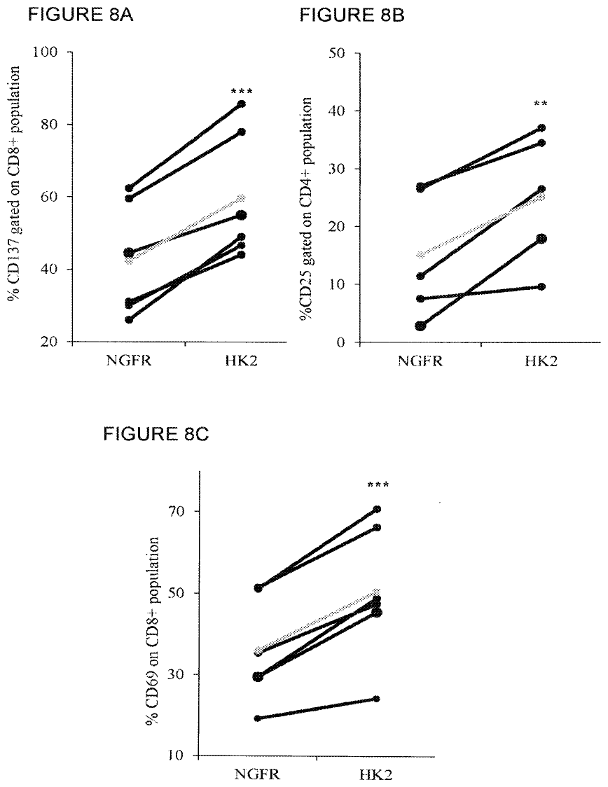

To further validate the results, the inventors evaluated activation markers in THE metabolic gene transduced T cells of the present disclosure. 4-1BB (CD137), an activation-induced costimulatory molecule, is an important regulator of immune responses. 4-1BB, a costimulatory molecule highly expressed on exhausted T cells, influences metabolic function. CD69 is an early lymphocyte activation marker that upregulates within hours, in response to TCR ligation. CD69 upregulation provides an additional co-stimulation to sustain the immune response. IL-2 cytokine is important in the anti-tumor response of immune cells [Sim G C and Radvanyi L. Cytokine Growth Factor Rev. Aug; 25(4):377-90 (2014)]. Its receptor is composed of three sub-units; IL-2R (CD25), IL-2R (CD122), and IL-2R (CD132). CD25 is not expressed by naïve T cells but its expression can be triggered rapidly by TCR and co-stimulatory signals. Since high levels of IL-2 secretion were observed in metabolic gene transduced T cells, CD137, CD69 and CD25 expression levels were evaluated in modified T cells. Indeed, an increase in the percentage of cells expressing the T-cell activation markers was detected in all the metabolic gene transduced T cells (FIGS. 8 to 11).

Specifically, HK2 and NGFR transduced cells were cultured following OKT3 activation, and the expression levels of the markers CD137 (FIG. 8A) and CD25 (FIG. 8B) was evaluated in the CD8 positive population and the expression levels of CD69 (FIG. 8C) was evaluated in the CD4 positive population.

The percentage of positive cells of CD137 was increased from a mean of 60% in HK2 transduced cells compared to a mean of 42% in the control NGFR cells (FIG. 8A, dark gray line). For CD69 expression, an increase from a mean of 50% in HK2 transduced cells compared to 36% in the control NGFR cells (FIG. 8C, light gray line) and for CD25 expression, an increase from a mean of 25% of CD25 in HK2 transduced cells compared to 15% in the NGFR cells (FIG. 8B, light line).

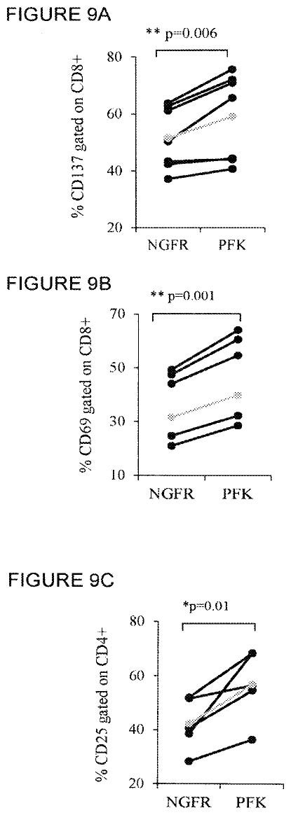

The percentage of positive cells of CD137 was increased from a mean of 59% in PFK transduced cells compared to a mean of 52% in the control NGFR cells (FIG. 9A). For CD69 expression, an increase from a mean of 40% in PFK transduced cells compared to 32% in the control NGFR cells (FIG. 9B) and for CD25 expression, an increase from a mean of 57% of CD25 in PFK transduced cells compared to 42% in the NGFR cells (FIG. 9C).

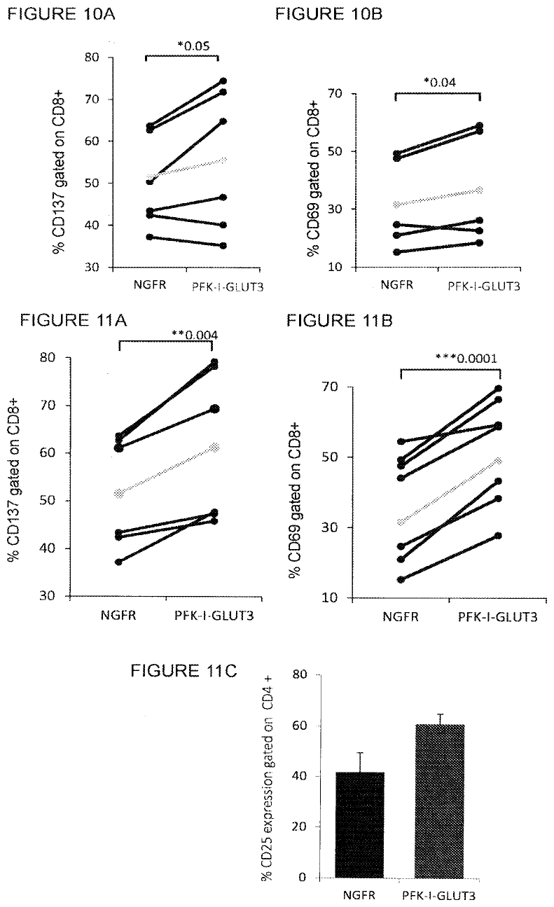

The percentage of positive cells of CD137 was increased from a mean of 56% in GLUT3 transduced cells compared to a mean of 52% in the control NGFR cells (FIG. 10A).

For CD69 expression, an increase from a mean of 41% in GLUT3 transduced cells compared to 32% in the control NGFR cells (FIG. 10B).

The percentage of positive cells of CD137 was increased from a mean of 61% in PFK-I-GLUT3 transduced cells compared to a mean of 52% in the control NGFR cells (FIG. 11A). For CD69 expression, an increase from a mean of 49% in PFK-I-GLUT3 transduced cells compared to 32% in the control NGFR cells (FIG. 11B) and for CD25 expression, an increase from a mean of 55% of CD25 in PFK-I-GLUT3 transduced cells compared to 42% in the NGFR cells (FIG. 11C).

### Example 3

### Increased Cytotoxicity and Reduced Exhaustion of Metabolically Engineered T Cells.

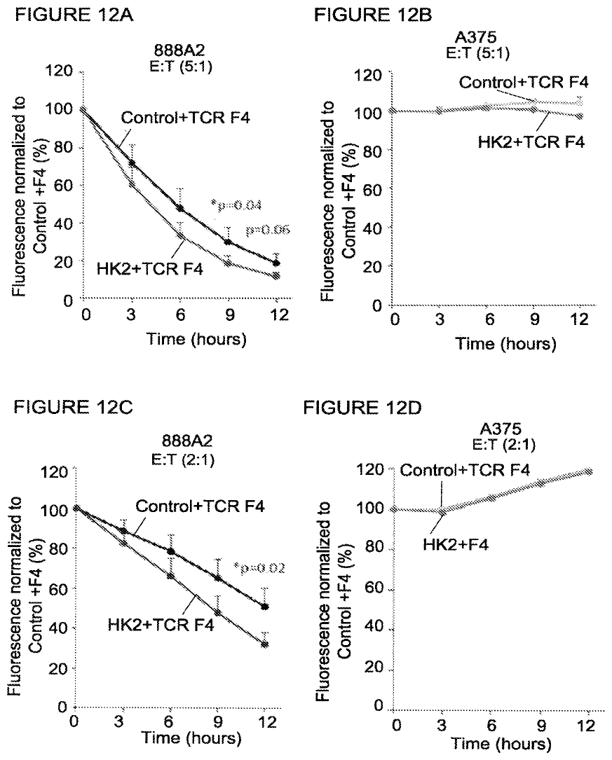

In vitro cytotoxicity assays provide an evaluation of T-cell lytic activity against target cells. Cytotoxicity HK2 transduced cells was assessed using the F4/F5 TCR system. PBLs co-transduced with F4 TCR and either HK2 or NGFR (control) and were incubated with target cell lines (negative target cells A375 and positive target cells 888A2 conjugated to mCherry) at a ratio of 5:1 and 2:1 (Effector cells:Target cells:). In both E:T ratios, HK2 engineered T cells mediated significant cytotoxicity against the positive target cells 888A2, when compared to control T cells (Control+TCR F4) after 9 hours (FIGS. 12A, 12C, HK2 mediated more cytotoxicity than control in E:T ratio of 5:1, and approximately 1.6 with a ratio of 1:2, compared to control (Empty+F4). No cytotoxicity was observed in the negative target cells A375 (FIG. 12B, 12D).

It is known that one mechanism for tumors to evade the immune system to survive in the host is by promoting T cell exhaustion. T cells that become exhausted had a reduced capacity to survive, proliferate and secrete cytokines. T cell dysfunction is marked by the progressive acquisition of inhibitory receptors (IRs), including lymphocyte activation gene-3 (LAG-3), and T cell immunoglobulin and mucin domain containing protein 3 (TIM3). These IRs alter T cell responses against tumors in part by perturbing their metabolism (Rivera G O. R, et al., Frontier in Immunology, 2021, Vol 12. Pages 1-17).

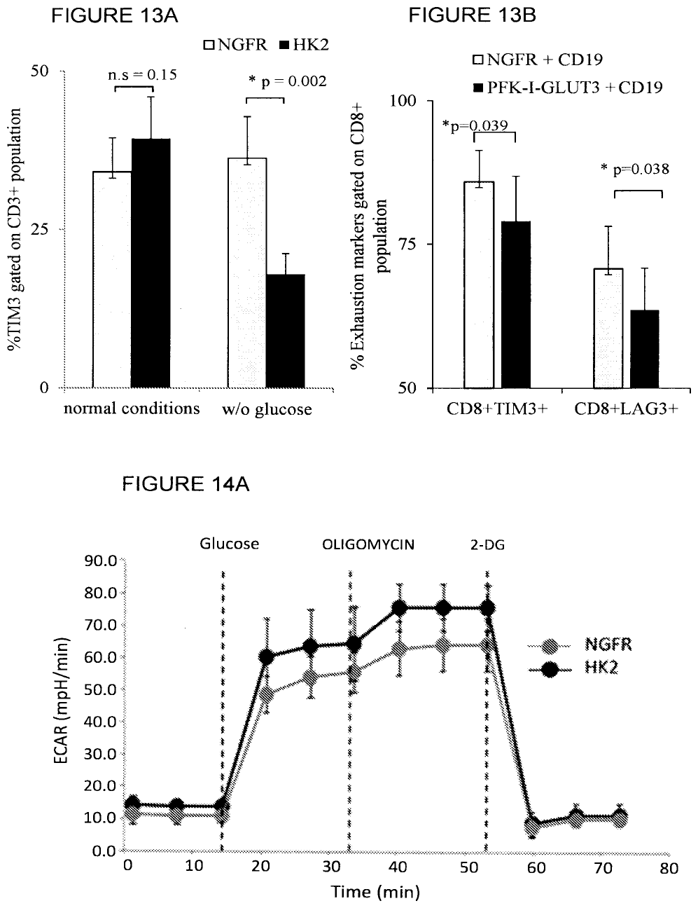

As mentioned in the introduction, microenvironment conditions are inconvenient for T cell survival and to maintain their effector function. T cells exhaustion following stimulation, a lack of positive co-stimulation and a constant immunosuppressive tumor microenvironment (TME) exposure could impair their anti-tumor function. To reproduce the TME, the inventors measured T cells exhaustion in normal and glucose-free conditions. TIM3 expression was measured in HK2 modified T-cells because it is known that TIM3 inhibits glycolysis in T cells through the HK2 enzyme [Lim, A. R., ibid. Liu C, Li H, Huang H, Zheng P, Li Z. The Correlation of HK2 Gene Expression with the Occurrence, Immune Cell Infiltration, and Prognosis of Renal Cell Carcinoma. Dis Markers. 2022 Feb. 27; 2022:1452861. doi: 10.1155/2022/1452861. PMID: 35265223; PMCID: PMC8898847. Lee, M. J., Yun, S. J., Lee, B. et al. Association of TIM-3 expression with glucose metabolism in Jurkat T cells. BMC Immunol 21, 48 (2020). https://doi.org/10.1186/s12865-020-00377-6]. In normal conditions, no significant change was observed between HK2-overexpressing T cells and control cells (FIG. 13A). In conditions without glucose, HK2 modified T-cells highly deregulate TIM3 expression (18% in the HK2 group vs. 36% in the control group (FIG. 13A).

Further, the inventors examined T cells exhaustion at a later day (between day 19-25) by measuring exhaustion markers in PFK-IRES-GLUT3 construct (also denoted herein as PFK-I-GLUT3 or PFK-GLUT3), transduced T-cells. By flow cytometry, TIM3 and LAG3 levels were measured in modified T-cells expressing PFK-I-GLUT3, using anti-LAG3 and anti-TIM3 antibodies (FIG. 13B). This assay was chosen in the CAR-CD19-BBz system since it was needed to grow cells for a long time and it is known that T cells expressing CAR CD19-BBz have a long lifespan [Kawalekar O U., Immunity. 16; 44(2):380-90 (2016)]. A higher percent of TIM3 and LAG3 was observed in the control (86% and 71% respectively) than in the PFK-I-GLUT3 group (71% and 64% respectively, FIG. 13B). The inventors concluded that PFK-I-GLUT3 decreases inhibitory receptors expression.

### Example 4

### Characterization of Metabolism Features in Engineered T Cells

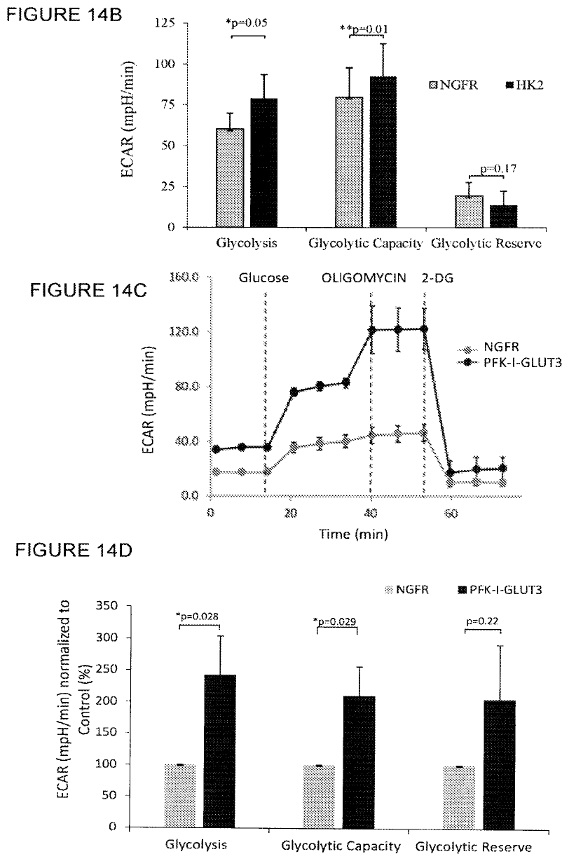

Measuring extracellular flux is a common method to evaluate changes in cellular metabolism, including glycolysis. Extracellular flux analysis involves measuring the rate of oxygen consumption and extracellular acidification, which are indicators of mitochondrial respiration and glycolysis, respectively. In modified T-cells, changes in extracellular flux can indicate an increase in glycolysis as a result of genetic modifications or other interventions. This approach can provide valuable insights into the metabolic changes that occur in engineered T-cells and how these changes may impact T-cell function. Indeed, HK2 overexpression led to increasing glycolysis as measured by extracellular acidification rate (ECAR, FIG. 14A). HK2 overexpression in T-cells increased glycolysis (78% in the HK2 group vs. 60% in the control group), glycolytic capacity (93% in the HK2 group vs. 80% in the control group) but not glycolytic reserve (19% in the HK2 group vs. 14% in the control group) compared to control (FIG. 14B). Similarly, PFK-I-GLUT3 overexpression led to increasing glycolysis as measured by ECAR (FIG. 14C). PFK-I-GLUT3 overexpressing T-cells increased glycolysis (2.4 times more in the PFK-I-GLUT3 group than in the control group), glycolytic capacity (2.1 times more in the PFK-I-GLUT3 group than in the control group) and glycolytic reserve (2 times more in the PFK-I-GLUT3 group than the control group) compared to control (FIG. 14D).

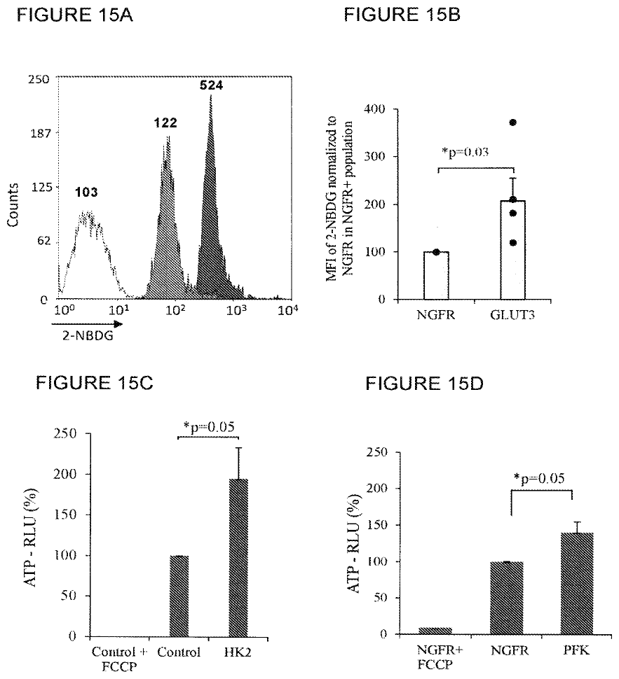

Since GLUT3 has a key role in glucose uptake in the cell, and it is known that glucose uptake could be diminished in the TME and affects T cells activation and proliferation [Dunn A F., Anal Chim Acta. 2; 1141:47-56 (2021)]. The inventors thus hypothesized that overexpressing GLUT3 transporter in T-cells could highly enhance glucose uptake in engineered T-cells. Accordingly, GLUT3 overexpressing T cells and NGFR transduced T cells (control) were starved and treated with the glucose analog 2-[N-(7-nitrobenz-2-oxa-1,3-dia-xol-4-yl) amino]-2-deoxyglucose (2-NBDG) [Dunn A F. Ibid.; Dunn A F., Anal Chim Acta. 2; 1141:47-56 (2021)] and the glucose uptake was quantified by flow cytometry. As shown in FIG. 15A-15B, glucose uptake was highly increased in GLUT3 transduced T-cells compared to the control NGFR transduced T-cells (MFI of 524 in the GLUT3 group compared to 122 in the control group).

Adenosine 5′-triphosphate (ATP) is the chemical energy for cellular metabolism and is often referred to as “energy money” of the cell. ATP is produced in living during glycolysis and OXYPHOS and consumed in cellular processes including biosynthetic reactions, motility and cell division. It is a key indicator of cellular activity and can also give a measure of cell viability. HK2 transduced cells or PFK transduced cells and the corresponding or control cells (transduced with empty vector), were incubated for 2 hours and the ATP production was measured. An improvement in ATP production of 95% in HK2 transduced T cells (FIG. 15C) and of 40% in PFK transduced T cells (FIG. 15D) was observed.

As indicated, T cells use different metabolic pathways based on their differentiation and memory status. Resting T cells largely depended on oxidative phosphorylation (OXPHOS) to survive. And they shift from OXPHOS to avid glycolysis and amino acid consumption upon TCR-mediated recognition of antigen (Rivera G O. R, et al., Frontier in Immunology, 2021, Vol 12. Pages 1-17).

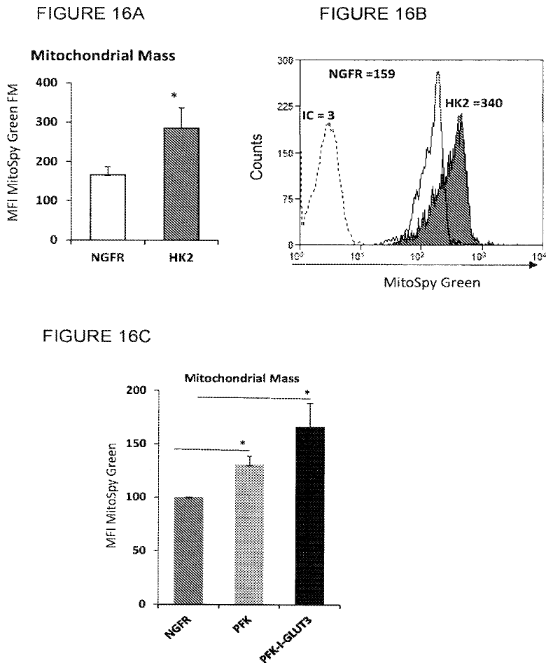

As shown in FIG. 14, metabolic gene transduced T cells increased glycolysis. Accordingly, the inventors proceeded to examine whether overexpression of glycolysis genes could also increase OXPHOS, and thus, mitochondrial mass in the modified T-cells was next examined. An increase in mitochondrial mass could be explained by an increase in mitochondrial fusion. Enhanced mitochondrial fusion highly improves OXPHOS and ATP production [Yao C H. Elife.; 8:e41351 (2019)]. For direct testing mitochondrial mass, the cells were stained with a mitochondrial probe, MitoSpy Green FM which binds to the mitochondrial membrane and thus enable evaluation of mitochondrial mass by flow cytometry (by MFI). Indeed, in HK2 transduced cells and PFK—I-NGFR transduced cells the mitochondrial mass was increased compared to the mitochondrial mass in the corresponding control cells. As shown in FIG. 16A-16B, the MFI of HK2 modified T-cells was 285 compared to 166 in control NGFR-transduced cells.

Similar results were obtained in the combined co-expression of PFK and GLUT3, in which the MFI of PFK-I-GLUT3 modified T-cells was 166 compared to 131 for PFK only and 100 in control NGFR-transduced cells, indicating of an increased mitochondrial mass (FIG. 16C).

### Example 5

### Improved Anti-Tumor Activity of Metabolically Transduced T-Cells In Vivo

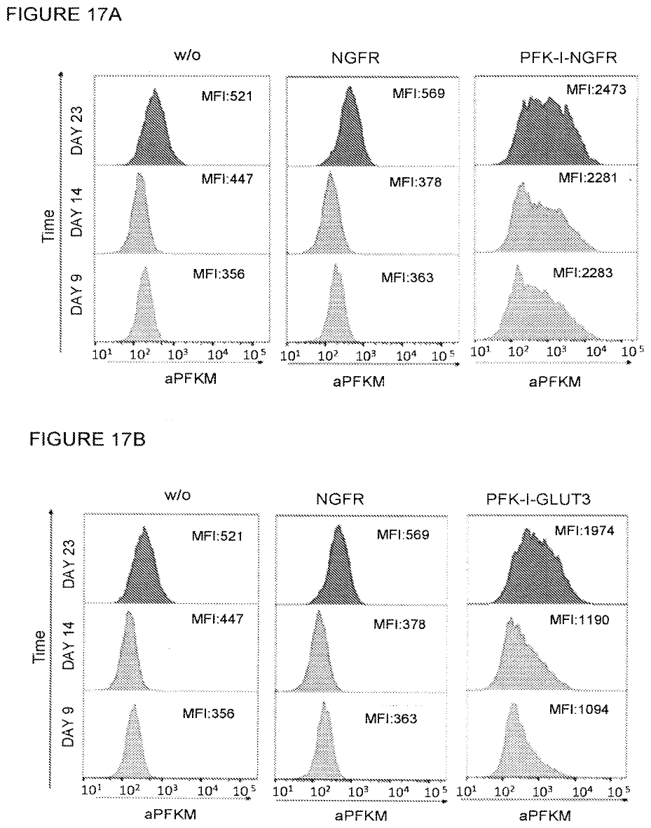

To further validate the results and the hypothesis that PFK transduced T cells undergo positive selection over time, PFK enzyme levels were measured every week, in untransduced T cells, NGFR, PFK and PFK-I-GLUT3 transduced T cells, by flow cytometry using anti-PFKM mAb and intracellular staining which measures the PFK enzyme level (FIG. 17).

An increase in PFK enzyme expression levels was observed over time. On day 9, the MFI in untransduced T cells group (w/o), was 356, and in the NGFR group was 363 (FIG. 17A). It was hypothesized that this is the basal level of the PFK enzyme in T cells after transduction. Moreover, the MFI in PFK transduced T cells was 2283 on day 9, indicating of high and successful transduction. At day 14, a small increase in the PFK level was observed in untransduced and NGFR groups (447 and 378 respectively), whereas the PFK levels in the PFK group remained stable (2281, FIG. 17A). At day 23, high increase in PFK levels was observed in all the groups (MFI of 521 in w/o, MFI of 569 in NGFR and MFI of 2473 in PFK, FIG. 17A). The inventors could thus conclude that cells undergo in some degree positive selection based on PFK enzyme expression.

The same assay to measure PFK enzyme levels was performed also in T-cells co-expressing PFK and GLUT3 (FIG. 17B). PFK levels were measured in untransduced T cells, NGFR and PFK-I-GLUT3 transduced T cells, by flow cytometry. Similar results were observed showing an increase in PFK enzyme level with time (FIG. 17B).

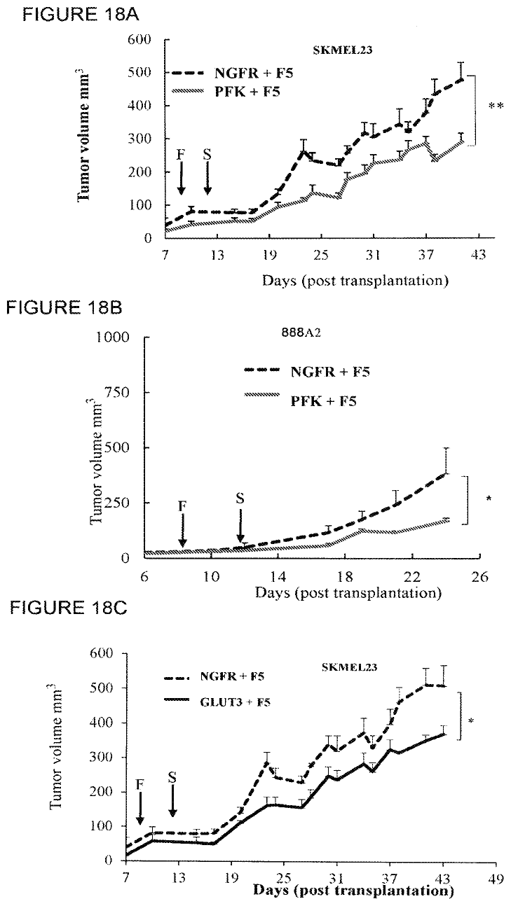

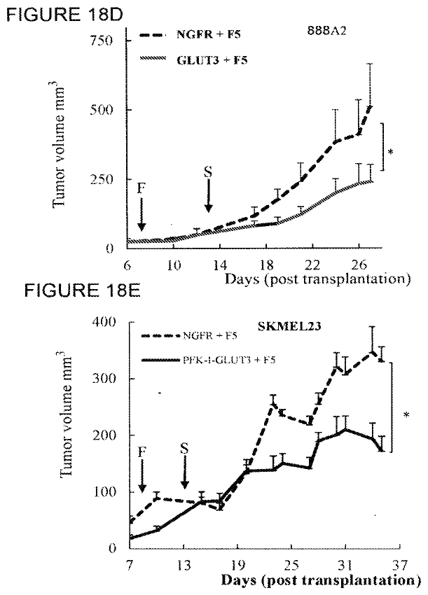

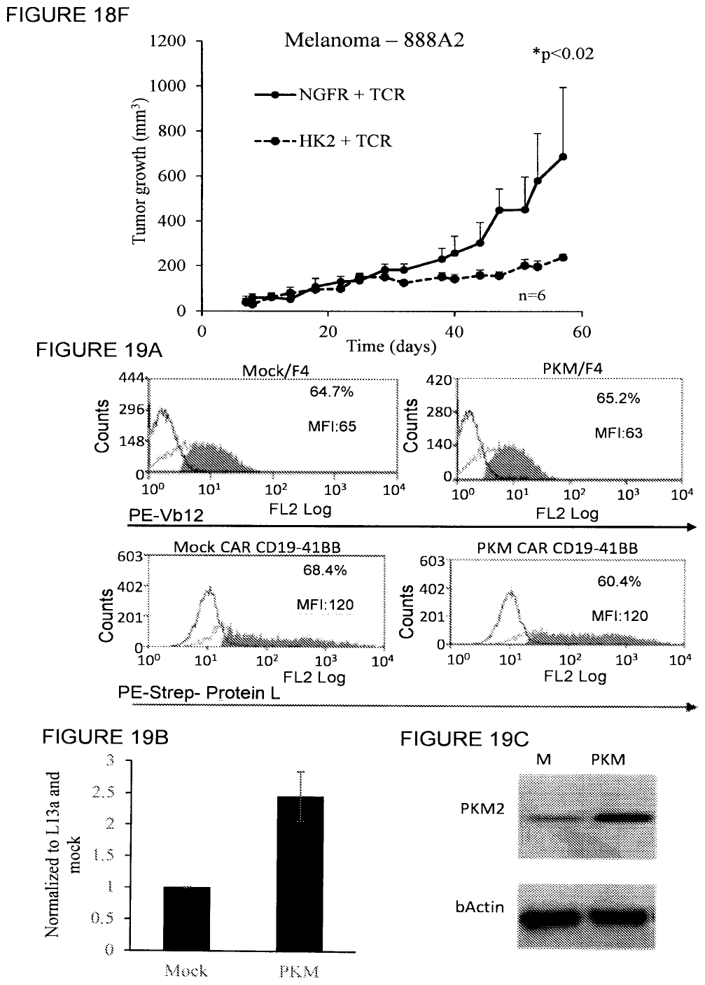

Next, the effect of metabolically transduced T-cells on tumor growth in vivo was tested. First an in vivo assay via an established tumor assay was performed to test the effect of PFK on tumor growth. Two million SKMEL23 (FIG. 18A) or 888A2 melanoma tumor cell lines (FIG. 18B) were injected to mice. One week after tumor establishment, two injections of either F5 TCR PFK modified T-cells or control F5 TCR NGFR modified cells were performed (on day 7-8-5×10⁶ engineered T cells and on day 12-13-10×10⁶ engineered T cells) and tumor growth was monitored for 6-8 weeks with a caliper.

As shown in FIG. 18A, T cells engineered to express PFK mediated a delay in tumor growth, exemplifying an improved anti-tumor function. In SKMEL23 tumor model, 43 days post transplantation of modified T-cells, tumor size in mice treated with PFK-transduced T cells was 294 mm³ versus 490 mm³ in mice treated with NGFR-transduced T cells (FIG. 18A). In 888A2 tumor model, 24 days post transplantation of modified T-cells, the tumor size in mice treated with PFK-transduced T cells was 168 mm³ versus 384 mm³ in NGFR-transduced T cells (FIG. 18B).

Next, the effect of GLUT3 on tumor growth was tested in a similar manner as done for PFK using in vivo assays via an established tumor assay.

In SKMEL23 tumor model, 43 days post transplantation of modified T-cells, tumor size in mice treated with Glut3-transduced T cells was 370 mm³ versus 508 mm³ in mice treated with NGFR-transduced T cells (FIG. 18C). In 888A2 tumor model, 27 days post transplantation of modified T-cells, the tumor size in mice treated with Glut3-transduced T cells was 239 mm³ versus 511 mm³ in NGFR-transduced T cells (FIG. 18D).

In view of the promising results with both PFK and GLUT3 genes separately an in vivo assay (via an established tumor assay) was also performed with T-cells co-expressing PFK and GLUT3. Two million SKMEL23 melanoma tumor cells (FIG. 18E) were injected to mice. One week after tumor establishment, two injections of either F5 TCR PFK-I-GLUT3 modified T-cells or control F5-NGFR cells were performed (on day 8-5×10⁶ engineered T cells and on day 13-10×10⁶ engineered T cells) and tumor growth was monitored for 6-8 weeks with a caliper.

Forty-three days post transplantation of modified T-cells, tumor size in mice treated with PFK-I-GLUT3-transduced T cells was 173 mm³ versus 318 mm³ in mice treated with NGFR-transduced T cells (FIG. 18E). PFK-I-GLUT3 highly delayed tumor growth in mice and increase the anti-tumor function of T cells.

Similarly, the in vivo activity of T-cells modified with HK2 was also evaluated (FIG. 18F). NSG mice were injected with the melanoma tumor 888A2 and treated with T-cells expressing a MART-1 specific TCR and HK2 or NGFR (control). As seen in FIG. 18F, on day 57, HK2-engineered T-cells were able to cause significant tumor growth delay (tumor volume of 239 mm³ vs 686 mm³ for control; n=6, p<0.02).

### Example 6

### Improved Activation, Metabolic Features, Cytotoxicity and Anti-Tumor Activity in PKM Transduced T-Cells

Pyruvate kinase is the enzyme involved in the last step of glycolysis. It catalyzes the transfer of a phosphate group from phosphoenolpyruvate (PEP) to adenosine diphosphate (ADP), yielding one molecule of pyruvate and one molecule of ATP. Since PKM plays a major role in cell metabolism, the inventors proceeded to examine whether PKM-modified T-cells show improved features as the other glycolysis related genes.

To determine the anti-tumor efficacy of PKM-transduced T cells, inventors first evaluated the expression levels of the different transgenes. F4-TCR was equally transduced in both mock- (64%) and PKM- (65%) transduced T cells (FIG. 19A, upper panel). CAR CD19-41BB was also similarly expressed in mock- and PKM-transduced T cells (68% and 60%), respectively (FIG. 19A, lower panel). A 2.5-fold increase (p<0.05) in PKM expression was validated by both real-time-PCR normalized to the L13a housekeeping gene (FIG. 19B) and by Western Blot normalized to b-Actin (FIG. 19C).

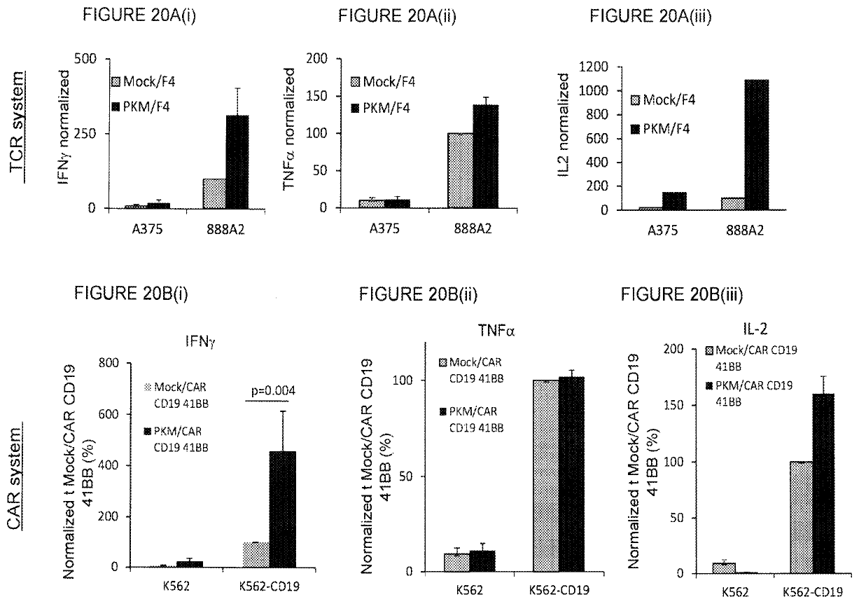

Next, the secretion of IFN-γ, TNF-α and IL-2 cytokines was evaluated in T-cells expressing PKM in both F4 TCR system in which transduced cells are incubated with positive 888A2 target cells or negative A375 cells (FIG. 20A) and in CAR CD19 41BB system in which transduced cells are incubated with K562-CD19 target cells or negative K562 cells (FIG. 20B). In both systems, PKM-transduced T cells have a superiority in secretion of IFN γ (2.5-4 fold increase, p<0.005), TNFα (1.1-1.3 fold increase, p<0.005) and IL2 (1.5 fold increase, p<0.05) when normalized to mock-transduced T cells.

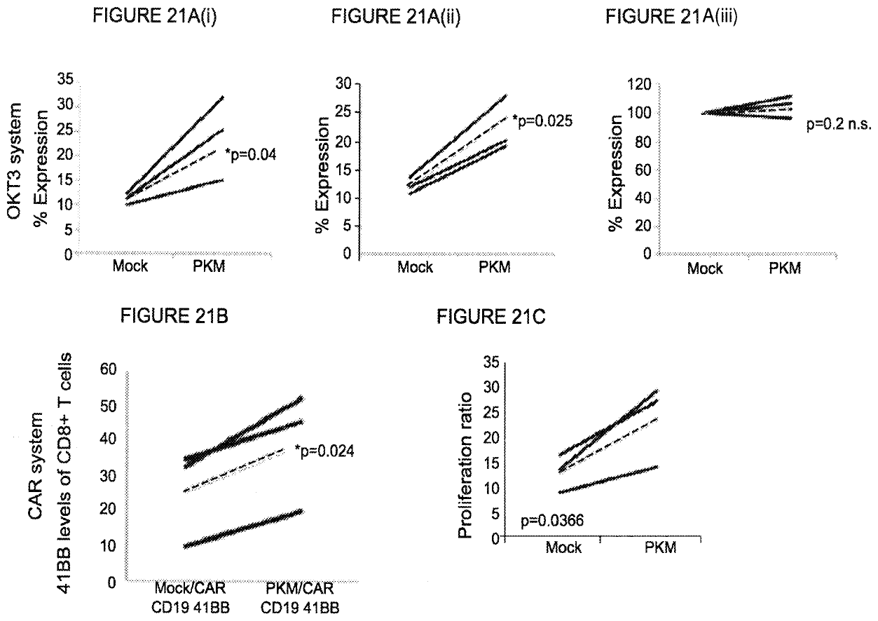

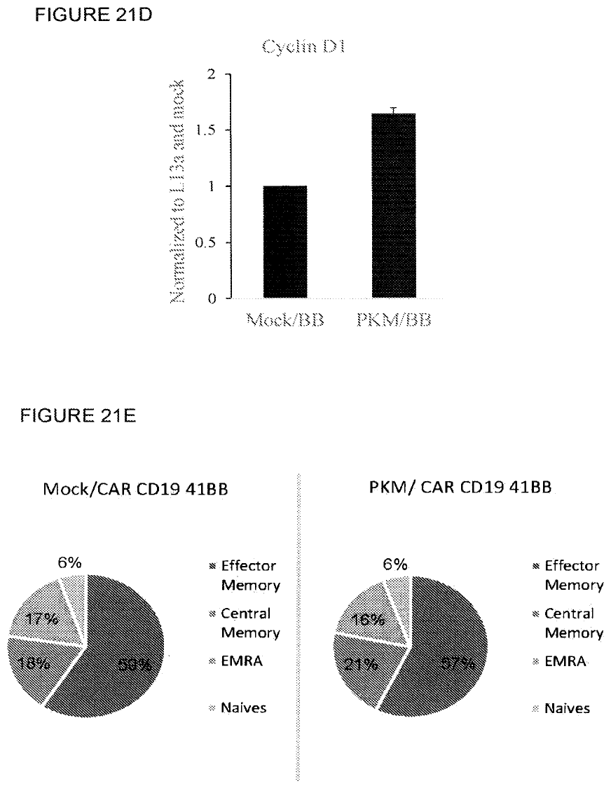

Next, the phenotype profile of PKM-induced T cells was evaluated in two different activation system; OKT3 or CAR systems. As shown FIG. 21A, PKM overexpression induced statistically significant higher expression of both 41BB (up to 20%, p=0.04) and CD25 (up to 15%, p=0.025) activation markers, in OKT3-induced system, as compared to mock-transduced T cells. CD69 expression was unaffected (p=0.2). Higher expression of 41BB activation marker was also observed in CAR system (15% increase on average, p=0.024) (FIG. 21B). These results support the fact that PKM induced a higher activation state in these T cells. Previous studies have shown that PKM2 is subjected to allosteric regulation in which PKM2 switches from a tetrameric (cytosolic localization) to a monomeric/dimeric state (nuclear localization). In the nucleus, PKM2 couples with transcription factors thus supporting, among others, cell cycle progression, proliferation and the Warburg effect. Indeed, PKM-induced T cells had twice as much proliferation rate as compared to their counterpart mock-T cells (FIG. 21C; 23 vs 12 proliferation ratio, p=0.036, respectively) results which were reinforced by 1.6 higher levels (p=0.03) of the Cyclin D1 gene (FIG. 21D). Still further, T-cells can exist in 4 different stage: Naïve (TN), effector memory (TEM), central memory (TCM) and EMRA (effector memory—RA+ indicated as “TE” in the figure). As shown in FIG. 21E, PKM-induced T cells have a slightly higher central memory phenotype (20% p=0.05) compared to mock-transduced T cells (16%) thus supporting prolong survival rate of these T cells.

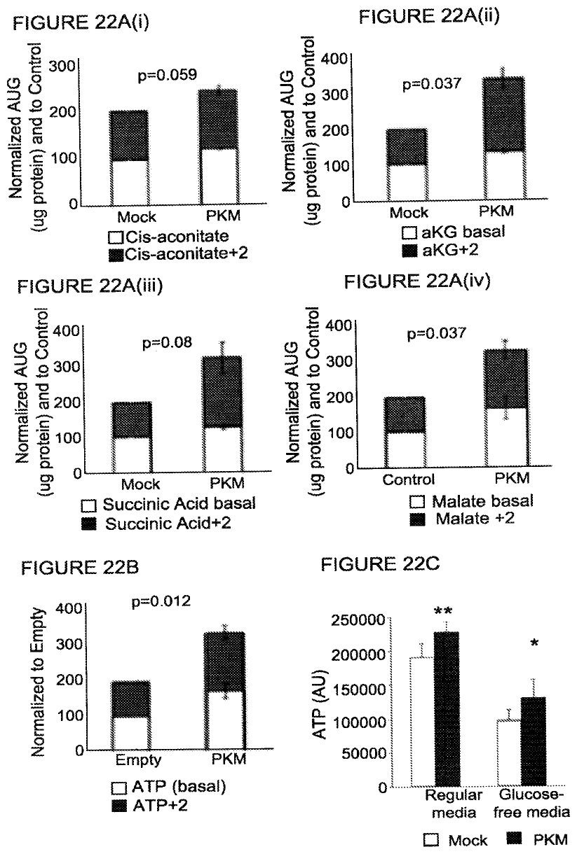

As indicated, PKM2 has two isomeric forms supporting two different cellular pathways; while in its dimeric form it translocated to the nucleus therefore supporting gene activation, in its tetrameric form it progresses through the TCA (tricarboxylic acid) cycle thus supporting ATP production, the final stage of glycolysis (Zahra K et al, Front. Oncol., (2020); Yang W and Lu Z, Cell Cycle. 12(19): 3154-3158 (2013); Venkatesh K. V., In: Dubitzky W., Wolkenhauer O., Cho K H., Yokota H. (eds) Encyclopedia of Systems Biology. Springer, New York, NY (2013)]. C-glucose tracing was used to evaluate the effect of PKM overexpression on TCA cycle progression. 4 major metabolites; Cis-aconitate (1.24 fold, p=0.059), alpha-ketoglutarate (aKG) (˜2 fold, p=0.037), Succinic acid (˜1.9 fold, p=0.08) and Malate (˜1.6 fold, p=0.037) were statistically elevated in PKM overexpression-compared to mock-transduced thus supporting progression through the TCA cycle both at the basal (white bars) as well as at M+2 levels (gray bars) (FIG. 22A). Additionally, net production of ATP was also approximately 1.7-fold higher (p=0.012) for the PKM transduced T cells compared to control, both at the basal levels (white bars) and M+2 (gray bars) (FIG. 22B).

The inventors also assessed ATP levels for T-cells engineered to PKM (or not) in medium with or without glucose. As seen in FIG. 22C, T-cells overexpressing PKM could produce more ATP than the control in both conditions.

Next the effect of PKM expression on tumor cell-cytotoxicity in vitro and antitumoral activity in vivo was investigated.

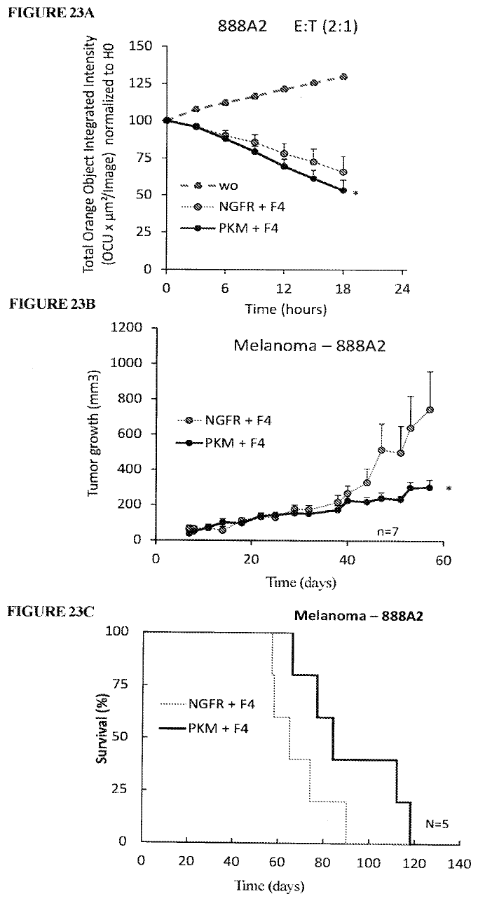

First, F4 TCR PKM- and F4 TCR mock-transduced T cells were co-culture with the positive target cells 888A2-mCherry melanoma cells at E:T ratio 2:1 and imaged every 3-hour using the Sartorius IncuCyte® system. The number of red fluorescent-888A2 cells decreased shortly upon administration of PKM-transduced T cells. 53% of alive cancer cells measured, as compared to 66% in mock-transduced group, a trend that increased consistently over 24 hr for the PKM-transduced cells (FIG. 23A, p<0.05). Next, the antitumoral activity of transduced T cells in tumor-bearing mice was tested.

NSG mice (n=7/group) were injected with 2×10⁶ 888A2 melanoma cells and on day 7 when tumors reached 5 mm, 5×10⁶ PKM- or mock-transduced T cells were injected I.V. (first I.V injection) into the mice followed by a second I.V injection a week later. Tumor volume increased steadily over time in the mock-treated group having 1.5-2 higher volume size as compared to that measured in PKM-treated group (FIG. 23B), and differences were statistically significant (303 mm3 vs 744 mm3, at day 57, p<0.05). Kaplan-Meier curves also show a better survival rate for the PKM-treated group compared to mock (FIG. 23C).

1-55. (canceled)

56. A genetically engineered hematopoietic cell/s or a cell population comprising at least one of said cell/s, wherein said genetically engineered cell/s comprises and/or expresses at least one nucleic acid sequence encoding at least one molecule involved directly or indirectly in at least one metabolic pathway, said cell optionally further expresses at least one receptor molecule.

57. The genetically engineered cell according to claim 56, wherein said hematopoietic cell is at least one lymphocyte, optionally, wherein said lymphocyte is of the T lineage.

58. The genetically engineered cell according to claim 56, wherein at least one of:
(a) said molecule involved directly or indirectly in at least one metabolic pathway is at least one of: a catabolic protein, an anabolic protein and/or an amphibolic protein;
(b) said molecule involved directly or indirectly in at least one metabolic pathway is a protein, and wherein said metabolic pathway is at least one of: glycolysis pathway, pentose phosphate pathway, fatty acid biosynthesis pathway, electron transport chain, and/or oxidative phosphorylation;
(c) said protein involved directly or indirectly in said at least one metabolic pathway is at least one of an enzymatic protein, a transporter protein, a structural protein, an adaptor protein and/or a protein participating in signal transduction related to said at least one metabolic pathway.

59. The genetically engineered cell according to claim 56, wherein
(a) said protein involved directly or indirectly in said at least one metabolic pathway is at least one of: Hexokinase (HK), Glucose Transporter Type 3 (GLUT3), Phosphofructokinase-1 (PFK-1), Pyruvate kinase (PK), Glucose-6-phosphate Isomerase (PGI), Fructose-bisphosphate aldolase (ALDO), Triosephosphate isomerase (TPI), Glyceraldehyde-3-phosphate dehydrogenase (GAPDH), Phosphoglycerate kinase (PGK), Phosphoglycerate mutase (PGM) and/or Phosphopyruvate hydratase (enolase); or
(b) said at least one protein involved directly or indirectly in said at least one metabolic pathway is at least one of: HK2, PFK-1, GLUT3, PK-M, and/or any combination thereof.

60. The genetically engineered cell according to claim 56, wherein at least one of:
(I) said cell further expressing at least one receptor molecule, said at least one receptor molecule comprises at least one target binding domain specific against at least one target antigen;
(II) said at least one receptor molecule is at least one of:
(a) a T-cell receptor (TCR) molecule specific for at least one target antigen; and/or
(b) a chimeric antigen receptor (CAR) molecule specific for at least one target antigen; and
(III) said at least one receptor molecule is a CAR molecule, said CAR molecule comprises:
at least one target-binding domain that specifically recognizes and binds at least one target antigen;
at least one hinge and at least one transmembrane domain; and
(iii) at least one intracellular T cell signal transduction domain.

61. The genetically engineered cell according to claim 60, wherein at least one of:
(a) said at least one target binding domain comprises at least one antibody or any antigen-binding fragment/s, portion/s or chimera/s thereof, specific for a target antigen;
(b) wherein the target antigen is an antigen associated with a pathologic disorder;
(c) said at least one target antigen is at least one of at least one tumor associated antigen (TAA), at least one tumor specific antigen, at least one neoantigen, at least one viral antigen, at least one bacterial antigen, at least one fungal antigen and/or at least one parasite antigen; and
(d) wherein TAA is a melanoma antigen, and/or a hematological malignancy antigen.

62. The genetically engineered cell according to claim 56, wherein expression of said protein involved directly or indirectly in said at least one metabolic pathway by said at least one genetically engineered cell, results in at least one of: (i) increased cytokine secretion; (ii) increased expression of activation markers; (iii) increased glycolysis; (iv) improvement in metabolic parameters; (v) increased cell survival; (vi) increased cytotoxicity; (vii) reduced expression of exhaustion markers; and/or (viii) increase in oxidative phosphorylation; by said genetically engineered cell; optionally, wherein said improvement in metabolic parameters comprises at least one of: increased ATP content, glucose uptake and/or mitochondrial mass.

63. A composition comprising at least one of:
(a) at least one genetically engineered cell/s according to claim 56, or a cell population comprising at least one of said genetically engineered cell/s, wherein said genetically engineered cell comprises and/or expresses at least one nucleic acid sequence encoding at least one molecule involved directly or indirectly in at least one metabolic pathway, said cell optionally further expresses at least one receptor molecule; and/or
(b) at least one nucleic acid sequence encoding said at least one molecule involved directly or indirectly in at least one metabolic pathway, or any cassette, vector and/or gene editing system comprising said nucleic acid sequence; said composition further comprise at least one of pharmaceutically acceptable carrier/s, diluent/s, excipient/s and additive/s.

64. A method for treating, preventing, ameliorating, inhibiting or delaying the onset of a pathologic disorder in a mammalian subject, said method comprising the step of administering to said subject an effective amount of at least one of:
(a) at least one genetically engineered hematopoietic cell/s or a cell population comprising at least one of said genetically engineered cell/s, wherein said genetically engineered cell comprises and/or expresses at least one nucleic acid sequence encoding at least one molecule involved directly or indirectly in at least one metabolic pathway, said cell optionally further expresses at least one receptor molecule;
(b) at least one nucleic acid sequence encoding said at least one molecule involved directly or indirectly in at least one metabolic pathway, or any cassette, vector and/or gene editing system comprising said nucleic acid sequence; and/or
(c) a composition comprising said genetically engineered cell/s of (a) and/or the nucleic acid sequence of (b).

65. The method according to claim 64, wherein said hematopoietic cell is at least one lymphocyte; optionally, wherein said lymphocyte is of the T lineage.

66. The method according to claim 64, wherein at least one of:
(a) said molecule involved directly or indirectly in at least one metabolic pathway is a catabolic protein, an anabolic protein or an amphibolic protein;
(b) said molecule involved directly or indirectly in at least one metabolic pathway is a protein, and wherein said metabolic pathway is at least one of: glycolysis pathway, pentose phosphate pathway, fatty acid biosynthesis pathway, electron transport chain, and/or oxidative phosphorylation;
(c) said protein involved directly or indirectly in said at least one metabolic pathway is at least one of an enzymatic protein, a transporter protein, a structural protein, an adaptor protein and a protein participating in signal transduction related to the metabolic pathway; and
(d) said protein involved directly or indirectly in said at least one metabolic pathway is at least one of HK, GLUT3, PFK-1, PKM, PGI, ALDO, TPI, GAPDH, PGK, PGM, and/or enolase; and

67. The method according to claim 66, wherein said protein involved directly or indirectly in said at least one metabolic pathway is at least one of HK2, PFK-1, GLUT3, PKM, and/or any combination thereof.

68. The method according to claim 64, wherein at least one of:
(a) said at least one receptor molecule comprises at least one target binding domain specific against at least one target antigen;
(b) said at least one receptor molecule is at least one TCR molecule;
(c) said at least one receptor molecule is a CAR molecule specific for at least one target antigen;
(d) said CAR molecule comprising:
(i) at least one target-binding domain that specifically recognizes and binds a target antigen;
(ii) at least one hinge and at least one transmembrane domain; and
(iii) at least one intracellular T cell signal transduction domain; and
(e) said at least one target binding domain comprises at least one antibody or any antigen-binding fragment/s, portion/s or chimera/s thereof, specific for a target antigen.

69. The method according to claim 68, wherein at least one of:
(a) the target antigen is an antigen associated with a pathologic disorder; and
(b) said target antigen is at least one of at least one TAA, at least one tumor specific antigen, at least one neoantigen, at least one viral antigen, at least one bacterial antigen, at least one fungal antigen and/or at least one parasite antigen.

70. The method according to claim 64, wherein said pathologic disorder is at least one of a proliferative disorder, a metabolic condition, an inflammatory disorder, an infectious disease caused by a pathogen, an autoimmune-disease, and a cardiovascular disease; optionally, said proliferative disorder is at least one hematological malignancy, and/or at least one solid tumor.

71. The method according to claim 64, wherein expression of said protein involved directly or indirectly in said at least one metabolic pathway by said at least one genetically engineered cell in said subject, results in at least one of:
(I) at least one of: (i) increased cytokine secretion; (ii) increased expression of activation markers; (iii) increased glycolysis; (iv) improvement in metabolic parameters; (v) increased survival; (vi) increased proliferation; and/or (vii) reduced expression of exhaustion markers; and/or (viii) increase in oxidative phosphorylation; by said genetically engineered cell; and/or
(II) at least one of: (i) increased survival; (ii) reduced relapse rate; and/or (iii) a long-term effect; in said subject.

72. The method according to claim 64, wherein the at least one genetically engineered cell or a cell population comprising at least one of said cell, are of an autologous or allogeneic source.

73. A method for improving activity and/or survival of at least one hematopoietic cell, the method comprising the step of contacting at least one cell with an effective amount of at least one nucleic acid sequence encoding at least one molecule involved directly or indirectly in at least one metabolic pathway, or any cassette, vector and/or gene editing system comprising said nucleic acid sequence, said cell optionally further expresses at least one receptor molecule; optionally, wherein said hematopoietic cell is at least one lymphocyte, optionally, said lymphocyte is of the T lineage.

74. The method according to claim 73, wherein expression of said protein involved directly or indirectly in said at least one metabolic pathway by said at least one cell of the T lineage results in at least one of: (i) increased cytokine secretion; (ii) increased expression of activation markers; (iii) increased glycolysis; (iv) improvement in metabolic parameters; (v) increased cell survival; (vi) increased cytotoxicity; (vii) reduced expression of exhaustion markers; (viii) increased proliferation; and/or (ix) increased oxidative phosphorylation, by said cell.

75. The method according to claim 73, wherein the step of contacting said cell/s with said at least one nucleic acid sequence, is performed in vivo, in vitro or ex vivo; wherein:
(a) contacting said cell with said at least one nucleic acid sequence, is performed in vivo in a subject suffering from at least one pathologic disorder, said contacting step comprises administering to said subject an effective amount of said nucleic acid sequence encoding at least one molecule involved directly or indirectly in at least one metabolic pathway, any cassette, vector and/or gene editing system comprising said nucleic acid sequence or any composition thereof, optionally, said at least one pathologic disorder is at least one of a proliferative disorder, a metabolic condition, an inflammatory disorder, an infectious disease caused by a pathogen, an autoimmune-disease, and a cardiovascular disease; or
(b) contacting said cell/s with said at least one nucleic acid sequence is performed in vitro or ex vivo, thereby obtaining genetically engineered cell/s, or a cell population comprising at least one of said cell/s; optionally, said cells are of autologous or allogeneic source.


SEQUENCE LISTING (41 sequences)

SEQ ID NO: 1 - protein, 917 aa, Homo sapiens
  source 1..917: note = Hexokinase 2 (HK2) CDS Uniprot P52789
MIASHLLAYFFTELNHDQVQKVDQYLYHMRLSDETLLEISKRFRKEMEKGLGATTHPTAAVKMLPTFVRSTPDGTEHGEFLALDLGGTNFRVLWVKVTDNGLQKVEMENQIYAIPEDIMRGSGTQLFDHIAECLANFMDKLQIKDKKLPLGFTFSFPCHQTKLDESFLVSWTKGFKSSGVEGRDVVALIRKAIQRRGDFDIDIVAVVNDTVGTMMTCGYDDHNCEIGLIVGTGSNACYMEEMRHIDMVEGDEGRMCINMEWGAFGDDGSLNDIRTEFDQEIDMGSLNPGKQLFEKMISGMYMGELVRLILVKMAKEELLFGGKLSPELLNTGRFETKDISDIEGEKDGIRKAREVLMRLGLDPTQEDCVATHRICQIVSTRSASLCAATLAAVLQRIKENKGEERLRSTIGVDGSVYKKHPHFAKRLHKTVRRLVPGCDVRFLRSEDGSGKGAAMVTAVAYRLADQHRARQKTLEHLQLSHDQLLEVKRRMKVEMERGLSKETHASAPVKMLPTYVCATPDGTEKGDFLALDLGGTNFRVLLVRVRNGKWGGVEMHNKIYAIPQEVMHGTGDELFDHIVQCIADFLEYMGMKGVSLPLGFTFSFPCQQNSLDESILLKWTKGFKASGCEGEDVVTLLKEAIHRREEFDLDVVAVVNDTVGTMMTCGFEDPHCEVGLIVGTGSNACYMEEMRNVELVEGEEGRMCVNMEWGAFGDNGCLDDFRTEFDVAVDELSLNPGKQRFEKMISGMYLGEIVRNILIDFTKRGLLFRGRISERLKTRGIFETKFLSQIESDCLALLQVRAILQHLGLESTCDDSIIVKEVCTVVARRAAQLCGAGMAAVVDRIRENRGLDALKVTVGVDGTLYKLHPHFAKVMHETVKDLAPKCDVSFLQSEDGSGKGAALITAVACRIREAGQR

SEQ ID NO: 2 - DNA, 2754 bp, Homo sapiens
  source 1..2754: mol_type = genomic DNA; note = Hexokinase -2 (HK2) CDS - GenBank: BC021116.1
atgattgcctcgcatctgcttgcctacttcttcacggagctcaaccatgaccaagtgcagaaggttgaccagtatctctaccacatgcgcctctctgatgagaccctcttggagatctctaagcggttccgcaaggagatggagaaagggcttggagccaccactcaccctactgcagcagtgaagatgctgcccacctttgtgaggtccactccagatgggacagaacacggagagttcctggctctggatcttggagggaccaacttccgtgtgctttgggtgaaagtaacggacaatgggctccagaaggtggagatggagaatcagatctatgccatccctgaggacatcatgcgaggcagtggcacccagctgtttgaccacattgccgaatgcctggctaacttcatggataagctacaaatcaaagacaagaagctcccactgggttttaccttctcgttcccctgccaccagactaaactagacgagagtttcctggtctcatggaccaagggattcaagtccagtggagtggaaggcagagacgttgtggctctgatccggaaggccatccagaggagaggggactttgatatcgacattgtggctgtggtgaatgacacagttgggaccatgatgacctgtggttatgatgaccacaactgtgagattggtctcattgtgggcacgggcagcaacgcctgctacatggaagagatgcgccacatcgacatggtggaaggcgatgaggggcggatgtgtatcaatatggagtggggggccttcggggacgatggctcgctcaacgacattcgcactgagtttgaccaggagattgacatgggctcactgaacccgggaaagcaactgtttgagaagatgatcagtgggatgtacatgggggagctggtgaggcttatcctggtgaagatggccaaggaggagctgctctttggggggaagctcagcccagagcttctcaacaccggtcgctttgagaccaaagacatctcagacattgaaggggagaaggatggcatccggaaggcccgtgaggtcctgatgcggttgggcctggacccgactcaggaggactgcgtggccactcaccggatctgccagatcgtgtccacacgctccgccagcctgtgcgcagccaccctggccgccgtgctgcagcgcatcaaggagaacaaaggcgaggagcggctgcgctctactattggggtcgacggttccgtctacaagaaacacccccattttgccaagcgtctacataagaccgtgcggcggctggtgcccggctgcgatgtccgcttcctccgctccgaggatggcagtggcaaaggtgcagccatggtgacagcagtggcttaccggctggccgatcaacaccgtgcccgccagaagacattagagcatctgcagctgagccatgaccagctgctggaggtcaagaggaggatgaaggtagaaatggagcgaggtctgagcaaggagactcatgccagtgcccccgtcaagatgctgcccacctacgtgtgtgctaccccggacggcacagagaaaggggacttcttggccttggaccttggaggaacaaatttccgggtcctgctggtccgtgttcggaatgggaagtggggtggagtggagatgcacaacaagatctacgccatcccgcaggaggtcatgcacggcaccggggacgagctctttgaccacattgtccagtgcatcgcggacttcctcgagtacatgggcatgaagggcgtgtccctgcctctgggttttaccttctccttcccctgccagcagaacagcctggacgagagcatcctcctcaagtggacaaaaggcttcaaggcatctggctgcgagggcgaggacgtggtgaccctgctgaaggaagcgatccaccggcgagaggagtttgacctggatgtggttgctgtggtgaacgacacagtcggaactatgatgacctgtggctttgaagaccctcactgtgaagttggcctcattgttggcacgggcagcaatgcctgctacatggaggagatgcgcaacgtggaactggtggaaggagaagaggggcggatgtgtgtgaacatggaatggggggccttcggggacaatggatgcctagatgacttccgcacagaatttgatgtggctgtggatgagctttcactcaaccccggcaagcagaggttcgagaaaatgatcagtggaatgtacctgggtgagattgtccgtaacattctcatcgatttcaccaagcgtggactgctcttccgaggccgcatctcagagcggctcaagacaaggggcatctttgaaaccaagttcttgtctcagattgagagtgactgcctggccctgctgcaagtccgagccatcctgcaacacttagggcttgagagcacctgtgacgacagcatcattgttaaggaggtgtgcactgtggtggcccggcgggcagcccagctctgtggcgcaggcatggccgctgtggtggacaggatacgagaaaaccgtgggctggacgctctcaaagtgacagtgggtgtggatgggaccctctacaagctacatcctcactttgccaaagtcatgcatgagacagtgaaggacctggctccgaaatgtgatgtgtctttcctgcagtcagaggatggcagcgggaagggggcggcgctcatcactgctgtggcctgccgcatccgtgaggctggacagcgatag

SEQ ID NO: 3 - protein, 780 aa, Homo sapiens
  source 1..780: note = Phosphofructokinase-1 (PFK-1)CDS P08237
MTHEEHHAAKTLGIGKAIAVLTSGGDAQGMNAAVRAVVRVGIFTGARVFFVHEGYQGLVDGGDHIKEATWESVSMMLQLGGTVIGSARCKDFREREGRLRAAYNLVKRGITNLCVIGGDGSLTGADTFRSEWSDLLSDLQKAGKITDEEATKSSYLNIVGLVGSIDNDFCGTDMTIGTDSALHRIMEIVDAITTTAQSHQRTFVLEVMGRHCGYLALVTSLSCGADWVFIPECPPDDDWEEHLCRRLSETRTRGSRLNIIIVAEGAIDKNGKPITSEDIKNLVVKRLGYDTRVTVLGHVQRGGTPSAFDRILGSRMGVEAVMALLEGTPDTPACVVSLSGNQAVRLPLMECVQVTKDVTKAMDEKKFDEALKLRGRSFMNNWEVYKLLAHVRPPVSKSGSHTVAVMNVGAPAAGMNAAVRSTVRIGLIQGNRVLVVHDGFEGLAKGQIEEAGWSYVGGWTGQGGSKLGTKRTLPKKSFEQISANITKFNIQGLVIIGGFEAYTGGLELMEGRKQFDELCIPFVVIPATVSNNVPGSDFSVGADTALNTICTTCDRIKQSAAGTKRRVFIIETMGGYCGYLATMAGLAAGADAAYIFEEPFTIRDLQANVEHLVQKMKTTVKRGLVLRNEKCNENYTTDFIFNLYSEEGKGIFDSRKNVLGHMQQGGSPTPFDRNFATKMGAKAMNWMSGKIKESYRNGRIFANTPDSGCVLGMRKRALVFQPVAELKDQTDFEHRIPKEQWWLKLRPILKILAKYEIDLDTSDHAHLEHITRKRSGEAAV

SEQ ID NO: 4 - DNA, 2343 bp, Homo sapiens
  source 1..2343: mol_type = genomic DNA; note = Phosphofructokinase-1 (PFK-1)CDS - BC021203
atgacccatgaagagcaccatgcagccaaaaccctggggattggcaaagccattgctgtcttaacctctggtggagatgcccaaggtatgaatgctgctgtcagggctgtggttcgagttggtatcttcaccggtgcccgtgtcttctttgtccatgagggttatcaaggcctggtggatggtggagatcacatcaaggaagccacctgggagagcgtttcgatgatgcttcagctgggaggcacggtgattggaagtgcccggtgcaaggactttcgggaacgagaaggacgactccgagctgcctacaacctggtgaagcgtgggatcaccaatctctgtgtcattgggggtgatggcagcctcactggggctgacaccttccgttctgagtggagtgacttgttgagtgacctccagaaagcaggtaagatcacagatgaggaggctacgaagtccagctacctgaacattgtgggcctggttgggtcaattgacaatgacttctgtggcaccgatatgaccattggcactgactctgccctgcatcggatcatggaaattgtagatgccatcactaccactgcccagagccaccagaggacatttgtgttagaagtaatgggccgccactgtggatacctggcccttgtcacctctctgtcctgtggggccgactgggtttttattcctgaatgtccaccagatgacgactgggaggaacacctttgtcgccgactcagcgagacaaggacccgtggttctcgtctcaacatcatcattgtggctgagggtgcaattgacaagaatggaaaaccaatcacctcagaagacatcaagaatctggtggttaagcgtctgggatatgacacccgggttactgtcttggggcatgtgcagaggggtgggacgccatcagcctttgacagaattctgggcagcaggatgggtgtggaagcagtgatggcacttttggaggggaccccagataccccagcctgtgtagtgagcctctctggtaaccaggctgtgcgcctgcccctcatggaatgtgtccaggtgaccaaagatgtgaccaaggccatggatgagaagaaatttgacgaagccctgaagctgagaggccggagcttcatgaacaactgggaggtgtacaagcttctagctcatgtcagacccccggtatctaagagtggttcgcacacagtggctgtgatgaacgtgggggctccggctgcaggcatgaatgctgctgttcgctccactgtgaggattggccttatccagggcaaccgagtgctcgttgtccatgatggtttcgagggcctggccaaggggcagatagaggaagctggctggagctatgttgggggctggactggccaaggtggctctaaacttgggactaaaaggactctacccaagaagagctttgaacagatcagtgccaatataactaagtttaacattcagggccttgtcatcattgggggctttgaggcttacacagggggcctggaactgatggagggcaggaagcagtttgatgagctctgcatcccatttgtggtcattcctgctacagtctccaacaatgtccctggctcagacttcagcgttggggctgacacagcactcaatactatctgcacaacctgtgaccgcatcaagcagtcagcagctggcaccaagcgtcgggtgtttatcattgagactatgggtggctactgtggctacctggctaccatggctggactggcagctggggccgatgctgcctacatttttgaggagcccttcaccattcgagacctgcaggcaaatgttgaacatctggtgcaaaagatgaaaacaactgtgaaaaggggcttggtgttaaggaatgaaaagtgcaatgagaactataccactgacttcattttcaacctgtactctgaggaggggaagggcatcttcgacagcaggaagaatgtgcttggtcacatgcagcagggtgggagcccaaccccatttgataggaattttgccactaagatgggcgccaaggctatgaactggatgtctgggaaaatcaaagagagttaccgtaatgggcggatctttgccaatactccagattcgggctgtgttctggggatgcgtaagagggctctggtcttccaaccagtggctgagctgaaggaccagacagattttgagcatcgaatccccaaggaacagtggtggctgaaactgaggcccatcctcaaaatcctagccaagtacgagattgacttggacacttcagaccatgcccacctggagcacatcacccggaagcggtccggggaagctgccgtctaa

SEQ ID NO: 5 - protein, 496 aa, Homo sapiens
  source 1..496: note = Glucose Transporter Type 3 (GLUT3) CDS P11169
MGTQKVTPALIFAITVATIGSFQFGYNTGVINAPEKIIKEFINKTLTDKGNAPPSEVLLTSLWSLSVAIFSVGGMIGSFSVGLFVNRFGRRNSMLIVNLLAVTGGCFMGLCKVAKSVEMLILGRLVIGLFCGLCTGFVPMYIGEISPTALRGAFGTLNQLGIVVGILVAQIFGLEFILGSEELWPLLLGFTILPAILQSAALPFCPESPRFLLINRKEEENAKQILQRLWGTQDVSQDIQEMKDESARMSQEKQVTVLELFRVSSYRQPIIISIVLQLSQQLSGINAVFYYSTGIFKDAGVQEPIYATIGAGVVNTIFTVVSLFLVERAGRRTLHMIGLGGMAFCSTLMTVSLLLKDNYNGMSFVCIGAILVFVAFFEIGPGPIPWFIVAELFSQGPRPAAMAVAGCSNWTSNFLVGLLFPSAAHYLGAYVFIIFTGFLITFLAFTFFKVPETRGRTFEDITRAFEGQAHGADRSGKDGVMEMNSIEPAKETTTNV

SEQ ID NO: 6 - DNA, 1491 bp, Homo sapiens
  source 1..1491: mol_type = genomic DNA; note = Glucose Transporter Type 3 (GLUT3) CDS - NM_006931.2
atggggacacagaaggtcaccccagctctgatatttgccatcacagttgctacaatcggctctttccaatttggctacaacactggggtcatcaatgctcctgagaagatcataaaggaatttatcaataaaactttgacggacaagggaaatgccccaccctctgaggtgctgctcacgtctctctggtccttgtctgtggccatattttccgtcgggggtatgatcggctccttttccgtcggactcttcgtcaaccgctttggcaggcgcaattcaatgctgattgtcaacctgttggctgtcactggtggctgctttatgggactgtgtaaagtagctaagtcggttgaaatgctgatcctgggtcgcttggttattggcctcttctgcggactctgcacaggttttgtgcccatgtacattggagagatctcgcctactgccctgcggggtgcctttggcactctcaaccagctgggcatcgttgttggaattctggtggcccagatctttggtctggaattcatccttgggtctgaagagctatggccgctgctactgggttttaccatccttcctgctatcctacaaagtgcagcccttccattttgccctgaaagtcccagatttttgctcattaacagaaaagaagaggagaatgctaagcagatcctccagcggttgtggggcacccaggatgtatcccaagacatccaggagatgaaagatgagagtgcaaggatgtcacaagaaaagcaagtcaccgtgctagagctctttagagtgtccagctaccgacagcccatcatcatttccattgtgctccagctctctcagcagctctctgggatcaatgctgtgttctattactcaacaggaatcttcaaggatgcaggtgttcaagagcccatctatgccaccatcggcgcgggtgtggttaatactatcttcactgtagtttctctatttctggtggaaagggcaggaagaaggactctgcatatgataggccttggagggatggctttttgttccacgctcatgactgtttctttgttattaaaggataactataatgggatgagctttgtctgtattggggctatcttggtctttgtagccttctttgaaattggaccaggccccattccctggtttattgtggccgaactcttcagccagggcccccgcccagctgcgatggcagtggccggctgctccaactggacctccaacttcctagtcggattgctcttcccctccgctgctcactatttaggagcctacgtttttattatcttcaccggcttcctcattaccttcttggcttttaccttcttcaaagtccctgagacccgtggcaggacttttgaggatatcacacgggcctttgaagggcaggcacacggtgcagatagatctggaaaggacggcgtcatggagatgaacagcatcgagcctgctaaggagaccaccaccaatgtttaa

SEQ ID NO: 7 - protein, 531 aa, Homo sapiens
  source 1..531: note = Pyruvate kinase M (PKM) CDS AAA36449.1
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQNLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 8 - DNA, 1596 bp, Homo sapiens
  source 1..1596: mol_type = genomic DNA; note = Pyruvate kinase M (PKM) CDS GenBank: M23725.1
atgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggcccagcttcccgatcagtggagacgttgaaggagatgattaagtctggaatgaatgtggctcgtctgaacttctctcatggaactcatgagtaccatgcggagaccatcaagaatgtgcgcacagccacggaaagctttgcttctgaccccatcctctaccggcccgttgctgtggctctagacactaaaggacctgagatccgaactgggctcatcaagggcagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcagatgctggagagcatgatcaagaagccccgccccactcgggctgaaggcagtgatgtggccaatgcagtcctggatggagccgactgcatcatgctgtctggagaaacagccaaaggggactatcctctggaggctgtgcgcatgcagaacctgattgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggcaaggcccgaggcttcttcaagaagggagatgtggtcattgtgctgaccggatggcgccctggctccggcttcaccaacaccatgcgtgttgttcctgtgccgtga

SEQ ID NO: 9 - protein, 10 aa, synthetic construct
  source 1..10: note = MART-126-35
EAAGIGILTV

SEQ ID NO: 10 - DNA, 834 bp, synthetic construct
  source 1..834: mol_type = other DNA; note = CASSETTE encoding NGFR/ or NGFR CDS Truncated NGFR
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctag

SEQ ID NO: 11 - protein, 118 aa, Homo sapiens
  source 1..118: note = NP_005502.1 MART-1
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 12 - DNA, 357 bp, Homo sapiens
  source 1..357: mol_type = genomic DNA; note = MART-1 CCDS6466.1
atgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaa

SEQ ID NO: 13 - protein, 268 aa, synthetic construct
  source 1..268: note = TCR specific for MART-1 (termed F4) Alpha chain
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGGTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 309 aa, synthetic construct
  source 1..309: note = TCR specific for MART-1 (termed F4) Beta chain
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEVGVGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 15 - DNA, 807 bp, synthetic construct
  source 1..807: mol_type = other DNA; note = TCR specific for MART-1 (termed F4) Alpha chain
atgttgcttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggtgggaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaggtggccgggtttaatctgctcatgacgctgcggctgtggtccagctga

SEQ ID NO: 16 - DNA, 930 bp, synthetic construct
  source 1..930: mol_type = other DNA; note = TCR specific for MART-1 (termed F4) Beta Chain
atgggcacaaggttgttcttctatgtggccctttgtctcctgtggacaggacacatggatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgaggtaggggttgggcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 17 - protein, 273 aa, synthetic construct
  source 1..273: note = TCR specific for MART-1 (termed F5) Alpha chain
MLKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 308 aa, synthetic construct
  source 1..308: note = TCR specific for MART-1 (termed F5) Beta chain
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 19 - DNA, 822 bp, synthetic construct
  source 1..822: mol_type = other DNA; note = TCR specific for MART-1 (termed F5) Alpha chain
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacttcggaggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaggtggccgggtttaatctgctcatgacgctgcggctgtggtccagctga

SEQ ID NO: 20 - protein, 927 aa, synthetic construct
  source 1..927: note = TCR specific for MART-1 (termed F5) Beta chain
ATGAGAATCAGGCTCCTGTGCTGTGTGGCCTTTTCTCTCCTGTGGGCAGGTCCAGTGATTGCTGGGATCACCCAGGCACCAACATCTCAGATCCTGGCAGCAGGACGGCGCATGACACTGAGATGTACCCAGGATATGAGACATAATGCCATGTACTGGTATAGACAAGATCTAGGACTGGGGCTAAGGCTCATCCATTATTCAAATACTGCAGGTACCACTGGCAAAGGAGAAGTCCCTGATGGTTATAGTGTCTCCAGAGCAAACACAGATGATTTCCCCCTCACGTTGGCGTCTGCTGTACCCTCTCAGACATCTGTGTACTTCTGTGCCAGCAGCCTAAGTTTCGGCACTGAAGCTTTCTTTGGACAAGGCACCAGACTCACAGTTGTAGAGGACCTGAACAAGGTGTTCCCACCCGAGGTCGCTGTGTTTGAGCCATCAGAAGCAGAGATCTCCCACACCCAAAAGGCCACACTGGTGTGCCTGGCCACAGGCTTCTTCCCTGACCACGTGGAGCTGAGCTGGTGGGTGAATGGGAAGGAGGTGCACAGTGGGGTCAGCACGGACCCGCAGCCCCTCAAGGAGCAGCCCGCCCTCAATGACTCCAGATACTGCCTGAGCAGCCGCCTGAGGGTCTCGGCCACCTTCTGGCAGAACCCCCGCAACCACTTCCGCTGTCAAGTCCAGTTCTACGGGCTCTCGGAGAATGACGAGTGGACCCAGGATAGGGCCAAACCCGTCACCCAGATCGTCAGCGCCGAGGCCTGGGGTAGAGCAGACTGTGGCTTTACCTCGGTGTCCTACCAGCAAGGGGTCCTGTCTGCCACCATCCTCTATGAGATCCTGCTAGGGAAGGCCACCCTGTATGCTGTGCTGGTCAGCGCCCTTGTGTTGATGGCCATGGTCAAGAGAAAGGATTTCTGA

SEQ ID NO: 21 - protein, 490 aa, synthetic construct
  source 1..490: note = Chimeric antigen receptor (CAR) consisting of an anti-CD19 scFv (single chain variable fragment) coupled to the costimulatory signaling domain CD28
MGLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 1473 bp, synthetic construct
  source 1..1473: mol_type = other DNA; note = Chimeric antigen receptor (CAR) consisting of an anti-CD19 scFv (single chain variable fragment) coupled to the costimulatory signaling domain CD28
atggggctgctgctggtcacctccctgctgctgtgcgaactgcctcaccctgccttcctgctgattcccgacattcagatgactcagacaaccagctccctgtccgcctctctgggcgacagagtgacaatctcttgcagggccagccaggatatctccaagtatctgaactggtaccagcagaagcctgacggcaccgtgaagctgctgatctatcacacatctaggctgcacagcggagtgccatcccggttcagcggatccggatctggaaccgactactctctgacaatcagcaacctggagcaggaggatatcgccacctatttctgccagcagggcaataccctgccctacacatttggcggcggcacaaagctggagatcaccggcagcacatccggatctggcaagcctggatccggagagggatctaccaagggagaggtgaagctgcaggagtccggacctggcctggtggcaccaagccagtccctgtctgtgacctgtacagtgtccggcgtgtctctgcctgactacggcgtgagctggatcaggcagccacctaggaagggcctggagtggctgggcgtgatctggggctccgagacaacatactataatagcgccctgaagtccagactgaccatcatcaaggataacagcaagtcccaggtgtttctgaagatgaattccctgcagaccgacgatacagccatctactattgtgccaagcactactattacggcggctcctatgctatggactactggggccagggcacctctgtgacagtgtctagcgccgccgccatcgaagtgatgtatccacccccttacctggataacgagaagagcaatggcaccatcatccacgtgaagggcaagcacctgtgcccttctccactgttccccggccctagcaagcccttttgggtgctggtggtggtgggaggcgtgctggcctgttattctctgctggtgacagtggccttcatcatcttttgggtgaggagcaagcggagccggctgctgcacagcgactacatgaacatgaccccaagacggcccggccccacaagaaagcactatcagccatacgcaccaccaagggacttcgcagcctatagatccagggtgaagttttctcgcagcgccgatgcaccagcatatcagcagggacagaatcagctgtacaacgagctgaatctgggcaggcgcgaggagtacgacgtgctggataagaggagaggccgggatcccgagatgggaggcaagccaaggaggaagaaccctcaggagggcctgtataatgagctgcagaaggacaagatggccgaggcctacagcgagatcggcatgaagggagagcggagaaggggcaagggacacgatggcctgtaccagggcctgagcacagcaaccaaggacacttatgacgctctgcacatgcaggctctgccacccaggtaa

SEQ ID NO: 23 - protein, 486 aa, synthetic construct
  source 1..486: note = Chimeric antigen receptor (CAR) consisting of an anti-CD19 scFv (single chain variable fragment) coupled to the costimulatory signaling domain 4-1BB
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 1461 bp, synthetic construct
  source 1..1461: mol_type = other DNA; note = Chimeric antigen receptor (CAR) consisting of an anti-CD19 scFv (single chain variable fragment) coupled to the costimulatory signaling domain 4-1BB
atggccctgcctgtgaccgcactgctgctgcccctggctctgctgctgcacgctgcccgccccgacattcagatgactcagactacttcatctctgagcgcctccctgggcgacagagtgacaatctcttgcagggccagccaggatatctccaagtatctgaactggtaccagcagaagccagacggcacagtgaagctgctgatctatcacacctctaggctgcacagcggagtgccatcccggttctctggaagcggatccggaacagactactctctgaccatcagcaacctggagcaggaggatatcgccacctatttctgccagcagggcaatacactgccctacacctttggcggcggcacaaagctggagatcaccggcggcggcggctctggaggaggaggaagcggaggaggaggatccgaggtgaagctgcaggagagcggacctggcctggtggcaccatctcagagcctgtccgtgacatgtaccgtgagcggcgtgtccctgcctgattacggcgtgtcctggatcaggcagccacctaggaagggcctggagtggctgggcgtgatctggggctctgagacaacatactataattccgccctgaagtctagactgaccatcatcaaggacaactctaagagccaggtgttcctgaagatgaattccctgcagacagacgataccgccatctactattgcgccaagcactactattacggcggctcttatgctatggactactggggccagggcacaagcgtgaccgtgagctccaccacaacccctgcaccacgcccacccacaccagcacctaccatcgcaagccagccactgtccctgcggcccgaggcctgtaggccagcagcaggaggagcagtgcacaccaggggcctggactttgcctgcgatatctacatctgggcaccactggcaggaacatgtggcgtgctgctgctgagcctggtcatcaccctgtattgcaagagaggcaggaagaagctgctgtacatcttcaagcagcccttcatgcggcccgtgcagacaacccaggaggaggacggctgctcctgtaggttccctgaagaggaggagggaggatgtgagctgagggtgaagttttcccggtctgccgatgccccagcctatcagcagggccagaaccagctgtacaacgagctgaatctgggccggagagaggagtatgacgtgctggataagaggcgcggcagggaccccgagatgggaggcaagccccggagaaagaaccctcaggagggcctgtataatgagctgcagaaggacaagatggccgaggcctacagcgagatcggcatgaagggagagaggcgccggggcaagggacacgatggcctgtaccagggcctgtccacagcaaccaaagacacttacgacgcactgcacatgcaggctctgccaccaagataa

SEQ ID NO: 25 - DNA, 2757 bp, synthetic construct
  source 1..2757: mol_type = other DNA; note = HK CONSTRUCT
atgtcgattgcctcgcatctgcttgcctacttcttcacggagctcaaccatgaccaagtgcagaaggttgaccagtatctctaccacatgcgcctctctgatgagaccctcttggagatctctaagcggttccgcaaggagatggagaaagggcttggagccaccactcaccctactgcagcagtgaagatgctgcccacctttgtgaggtccactccagatgggacagaacacggagagttcctggctctggatcttggagggaccaacttccgtgtgctttgggtgaaagtaacggacaatgggctccagaaggtggagatggagaatcagatctatgccatccctgaggacatcatgcgaggcagtggcacccagctgtttgaccacattgccgaatgcctggctaacttcatggataagctacatatcaaagacaagaagctcccactgggttttaccttctcgttcccctgccaccagactaaactagacgagagtttcctggtctcatggaccaagggattcaagtccagtggagtggaaggcagagacgttgtggctctgatccggaaggccatccagaggagaggggactttgatatcgacattgtggctgtggtgaatgacacagttgggaccatgatgacctgtggttatgatgaccacaactgtgagattggtctcattgtgggcacgggcagcaacgcctgctacatggaagagatgcgccacatcgacatggtggaaggcgacgaggggcggatgtgtatcaatatggagtggggggccttcggggacgatggctcgctcaacgacattcgcactgagtttgaccaggagattgacatgggctcactgaacccgggaaagcaactgtttgagaagatgatcagtgggatgtacatgggggagctggtgaggcttatcctggtgaagatggccaaggaggagctgctctttggggggaagctcagcccagagcttctcaacaccggtcgctttgagaccaaagacatctcagacattgaaggggagaaggatggcatccggaaggcccgtgaggtcctgatgcggttgggcctggacccgactcaggaggactgcgtggccactcaccggatctgccagatcgtgtccacacgctccgccagcctgtgcgcagccaccctggccgccgtgctgcagcgcatcaaggagaacaaaggcgaggagcggctgcgctctactattggggtcgacggttccgtctacaagaaacacccccattttgccaagcgtctacataagaccgtgcggcggctggtgcccggctgcgatgtccgcttcctccgctccgaggatggcagtggcaaaggtgcagccatggtgacagcagtggcttaccggctggccgatcaacaccgtgcccgccagaagacattagagcatctgcagctgagccatgaccagctgctggaggtcaagaggaggatgaaggtagaaatggagcgaggtctgagcaaggagactcatgccagtgcccccgtcaagatgctgcccacctacgtgtgtgctaccccggacggcacagagaaaggggacttcttggccttggaccttggaggaacaaatttccgggtcctgctggtccgtgttcggaatgggaagtggggtggagtggagatgcacaacaagatctacgccatcccgcaggaggtcatgcacggcaccggggacgagctctttgaccacattgtccagtgcatcgcggacttcctcgagtacatgggcatgaagggcgtgtccctgcctctgggttttaccttctccttcccctgccagcagaacagcctggacgagagcatcctcctcaagtggacaaaaggcttcaaggcatctggctgcgagggcgaggacgtggtgaccctgctgaaggaagcgatccaccggcgagaggagtttgacctggatgtggttgctgtggtgaacgacacagtcggaactatgatgacctgtggctttgaagaccctcactgtgaagttggcctcattgttggcacgggcagcaatgcctgctacatggaggagatgcgcaacgtggaactggtggaaggagaagaggggcggatgtgtgtgaacatggaatggggggccttcggggacaatggatgcctagatgacttccgcacagaatttgatgtggctgtggatgagctttcactcaacccgggcaagcagaggttcgagaaaatgatcagtggaatgtacctgggtgagattgtccgtaacattctcatcgatttcaccaagcgtggactgctcttccgaggccgcatctcagagcggctcaagacaaggggcatctttgaaaccaagttcttgtctcagattgagagtgactgcctggccctgctgcaagtccgagccatcctgcaacacttagggcttgagagcacctgtgacgacagcatcattgttaaggaggtgtgcactgtggtggcccggcgggcagcccagctctgtggcgcaggcatggccgctgtggtggacaggatacgagaaaaccgtgggctggacgctctcaaagtgacagtgggtgtggatgggaccctctacaagctacatcctcactttgccaaagtcatgcatgagacagtgaaggacctggctccgaaatgtgatgtgtctttcctgcagtcagaggatggcagcgggaagggggcggcgctcatcactgctgtggcctgccgcatccgtgaggctggacagcgatag

SEQ ID NO: 26 - DNA, 4472 bp, synthetic construct
  source 1..4472: mol_type = other DNA; note = PFK IRES GLUT3 CONSTRUCT
atgtcgacccatgaagagcaccatgcagccaaaaccctggggattggcaaagccattgctgtcttaacctctggtggagatgcccaaggtatgaatgctgctgtcagggctgtggttcgagttggtatcttcaccggtgcccgtgtcttctttgtccatgagggttatcaaggcctggtggatggtggagatcacatcaaggaagccacctgggagagcgtttcgatgatgcttcagctgggaggcacggtgattggaagtgcccggtgcaaggactttcgggaacgagaaggacgactccgagctgcctacaacctggtgaagcgtgggatcaccaatctctgtgtcattgggggtgatggcagcctcactggggctgacaccttccgttctgagtggagtgacttgttgagtgacctccagaaagcaggtaagatcacagatgaggaggctacgaagtccagctacctgaacattgtgggcctggttgggtcaattgacaatgacttctgtggcactgatatgaccattggcactgactctgccctgcatcggatcatggaaattgtagatgccatcactaccactgcccagagccaccagaggacatttgtgttagaagtaatgggccgccactgtggatacctggcccttgtcacctctctgtcctgtggggccgactgggtttttattcctgaatgtccaccagatgacgactgggaggaacacctttgtcgccgactcagcgagacaaggacccgtggttctcgtctcaacatcatcattgtggctgagggtgcaattgacaagaatggaaaaccaatcacctcagaagacatcaagaatctggtggttaagcgtctgggatatgacacccgggttactgtcttggggcatgtgcagaggggtgggacgccatcagcctttgacagaattctgggcagcaggatgggtgtggaagcagtgatggcacttttggaggggaccccagataccccagcctgtgtagtgagcctctctggtaaccaggctgtgcgcctgcccctcatggaatgtgtccaggtgaccaaagatgtgaccaaggccatggatgagaagaaatttgacgaagccctgaagctgagaggccggagcttcatgaacaactgggaggtgtacaagcttctagctcatgtcagacccccggtatctaagagtggttcgcacacagtggctgtgatgaacgtgggggctccggctgcaggcatgaatgctgctgttcgctccactgtgaggattggccttatccagggcaaccgagtgctcgttgtccatgatggtttcgagggcctggccaaggggcagatagaggaagctggctggagctatgttgggggctggactggccaaggtggctctaaacttgggactaaaaggactctacccaagaagagctttgaacagatcagtgccaatataactaagtttaacattcagggccttgtcatcattgggggctttgaggcttacacagggggcctggaactgatggagggcaggaagcagtttgatgagctctgcatcccatttgtggtcattcctgctacagtctccaacaatgtccctggctcagacttcagcgttggggctgacacagcactcaatactatctgcacaacctgtgaccgcatcaagcagtcagcagctggcaccaagcgtcgggtgtttatcattgagactatgggtggctactgtggctacctggctaccatggctggactggcagctggggccgatgctgcctacatttttgaggagcccttcaccattcgagacctgcaggcaaatgttgaacatctggtgcaaaagatgaaaacaactgtgaaaaggggcttggtgttaaggaatgaaaagtgcaatgagaactataccactgacttcattttcaacctgtactctgaggaggggaagggcatcttcgacagcaggaagaatgtgcttggtcacatgcagcagggtgggagcccaaccccatttgataggaattttgccactaagatgggcgccaaggctatgaactggatgtctgggaaaatcaaagagagttaccgtaatgggcggatctttgccaatactccagattcgggctgtgttctggggatgcgtaagagggctctggtcttccaaccagtggctgagctgaaggaccagacagattttgagcatcgaatccccaaggaacagtggtggctgaaactgaggcccatcctcaaaatcctagccaagtacgagattgacttggacacttcagaccatgcccacctggagcacatcacccggaagcggtccggggaagctgccgtctaagcggccgctctagaactagtggatctccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatccatgcatgccgccatggggacacagaaggtcaccccagctctgatatttgccatcacagttgctacaatcggctctttccaatttggctacaacactggggtcatcaatgctcctgagaagatcataaaggaatttatcaataaaactttgacggacaagggaaatgccccaccctctgaggtgctgctcacgtctctctggtccttgtctgtggccatattttccgtcgggggtatgatcggctccttttccgtcggactcttcgtcaaccgctttggcaggcgcaattcaatgctgattgtcaacctgttggctgtcactggtggctgctttatgggactgtgtaaagtagctaagtcggttgaaatgctgatcctgggtcgcttggttattggcctcttctgcggactctgcacaggttttgtgcccatgtacattggagagatctcgcctactgccctgcggggtgcctttggcactctcaaccagctgggcatcgttgttggaattctggtggcccagatctttggtctggaattcatccttgggtctgaagagctatggccgctgctactgggttttaccatccttcctgctatcctacaaagtgcagcccttccattttgccctgaaagtcccagatttttgctcattaacagaaaagaagaggagaatgctaagcagatcctccagcggttgtggggcacccaggatgtatcccaagacatccaggagatgaaagatgagagtgcaaggatgtcacaagaaaagcaagtcaccgtgctagagctctttagagtgtccagctaccgacagcccatcatcatttccattgtgctccagctctctcagcagctctctgggatcaatgctgtgttctattactcaacaggaatcttcaaggatgcaggtgttcaagagcccatctatgccaccatcggcgcgggtgtggttaatactatcttcactgtagtttctctatttctggtggaaagggcaggaagaaggactctgcatatgataggccttggagggatggctttttgttccacgctcatgactgtttctttgttattaaaggataactataatgggatgagctttgtctgtattggggctatcttggtctttgtagccttctttgaaattggaccaggccccattccctggtttattgtggccgaactcttcagccagggcccccgcccagctgcgatggcagtggccggctgctccaactggacctccaacttcctagtcggattgctcttcccctccgctgctcactatttaggagcctacgtttttattatcttcaccggcttcctcattaccttcttggcttttaccttcttcaaagtccctgagacccgtggcaggacttttgaggatatcacacgggcctttgaagggcaggcacacggtgcagatagatctggaaaggacggcgtcatggagatgaacagcatcgagcctgctaaggagaccaccaccaatgtttaa

SEQ ID NO: 27 - DNA, 1596 bp, synthetic construct; identical to SEQ ID NO: 8
  source 1..1596: mol_type = other DNA; note = PKM CONSTRUCT

SEQ ID NO: 28 - DNA, 2346 bp, synthetic construct
  source 1..2346: mol_type = other DNA; note = PFK CONSTRUCT
atgtcgacccatgaagagcaccatgcagccaaaaccctggggattggcaaagccattgctgtcttaacctctggtggagatgcccaaggtatgaatgctgctgtcagggctgtggttcgagttggtatcttcaccggtgcccgtgtcttctttgtccatgagggttatcaaggcctggtggatggtggagatcacatcaaggaagccacctgggagagcgtttcgatgatgcttcagctgggaggcacggtgattggaagtgcccggtgcaaggactttcgggaacgagaaggacgactccgagctgcctacaacctggtgaagcgtgggatcaccaatctctgtgtcattgggggtgatggcagcctcactggggctgacaccttccgttctgagtggagtgacttgttgagtgacctccagaaagcaggtaagatcacagatgaggaggctacgaagtccagctacctgaacattgtgggcctggttgggtcaattgacaatgacttctgtggcactgatatgaccattggcactgactctgccctgcatcggatcatggaaattgtagatgccatcactaccactgcccagagccaccagaggacatttgtgttagaagtaatgggccgccactgtggatacctggcccttgtcacctctctgtcctgtggggccgactgggtttttattcctgaatgtccaccagatgacgactgggaggaacacctttgtcgccgactcagcgagacaaggacccgtggttctcgtctcaacatcatcattgtggctgagggtgcaattgacaagaatggaaaaccaatcacctcagaagacatcaagaatctggtggttaagcgtctgggatatgacacccgggttactgtcttggggcatgtgcagaggggtgggacgccatcagcctttgacagaattctgggcagcaggatgggtgtggaagcagtgatggcacttttggaggggaccccagataccccagcctgtgtagtgagcctctctggtaaccaggctgtgcgcctgcccctcatggaatgtgtccaggtgaccaaagatgtgaccaaggccatggatgagaagaaatttgacgaagccctgaagctgagaggccggagcttcatgaacaactgggaggtgtacaagcttctagctcatgtcagacccccggtatctaagagtggttcgcacacagtggctgtgatgaacgtgggggctccggctgcaggcatgaatgctgctgttcgctccactgtgaggattggccttatccagggcaaccgagtgctcgttgtccatgatggtttcgagggcctggccaaggggcagatagaggaagctggctggagctatgttgggggctggactggccaaggtggctctaaacttgggactaaaaggactctacccaagaagagctttgaacagatcagtgccaatataactaagtttaacattcagggccttgtcatcattgggggctttgaggcttacacagggggcctggaactgatggagggcaggaagcagtttgatgagctctgcatcccatttgtggtcattcctgctacagtctccaacaatgtccctggctcagacttcagcgttggggctgacacagcactcaatactatctgcacaacctgtgaccgcatcaagcagtcagcagctggcaccaagcgtcgggtgtttatcattgagactatgggtggctactgtggctacctggctaccatggctggactggcagctggggccgatgctgcctacatttttgaggagcccttcaccattcgagacctgcaggcaaatgttgaacatctggtgcaaaagatgaaaacaactgtgaaaaggggcttggtgttaaggaatgaaaagtgcaatgagaactataccactgacttcattttcaacctgtactctgaggaggggaagggcatcttcgacagcaggaagaatgtgcttggtcacatgcagcagggtgggagcccaaccccatttgataggaattttgccactaagatgggcgccaaggctatgaactggatgtctgggaaaatcaaagagagttaccgtaatgggcggatctttgccaatactccagattcgggctgtgttctggggatgcgtaagagggctctggtcttccaaccagtggctgagctgaaggaccagacagattttgagcatcgaatccccaaggaacagtggtggctgaaactgaggcccatcctcaaaatcctagccaagtacgagattgacttggacacttcagaccatgcccacctggagcacatcacccggaagcggtccggggaagctgccgtctaa

SEQ ID NO: 29 - protein, 558 aa, Homo sapiens
  source 1..558: note = Glucose-6-phosphate isomerase P06744
MAALTRDPQFQKLQQWYREHRSELNLRRLFDANKDRFNHFSLTLNTNHGHILVDYSKNLVTEDVMRMLVDLAKSRGVEAARERMFNGEKINYTEGRAVLHVALRNRSNTPILVDGKDVMPEVNKVLDKMKSFCQRVRSGDWKGYTGKTITDVINIGIGGSDLGPLMVTEALKPYSSGGPRVWYVSNIDGTHIAKTLAQLNPESSLFIIASKTFTTQETITNAETAKEWFLQAAKDPSAVAKHFVALSTNTTKVKEFGIDPQNMFEFWDWVGGRYSLWSAIGLSIALHVGFDNFEQLLSGAHWMDQHFRTTPLEKNAPVLLALLGIWYINCFGCETHAMLPYDQYLHRFAAYFQQGDMESNGKYITKSGTRVDHQTGPIVWGEPGTNGQHAFYQLIHQGTKMIPCDFLIPVQTQHPIRKGLHHKILLANFLAQTEALMRGKSTEEARKELQAAGKSPEDLERLLPHKVFEGNRPTNSIVFTKLTPFMLGALVAMYEHKIFVQGIIWDINSFDQWGVELGKQLAKKIEPELDGSAQVTSHDASTNGLINFIKQQREARVQ

SEQ ID NO: 30 - protein, 364 aa, Homo sapiens
  source 1..364: note = >sp|P04075|ALDOA_HUMAN Fructose-bisphosphate aldolase A OS=Homo sapiens OX=9606 GN=ALDOA PE=1 SV=2
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 31 - protein, 249 aa, Homo sapiens
  source 1..249: note = Triosephosphate isomerase (TPI) P60174
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 32 - protein, 335 aa, Homo sapiens
  source 1..335: note = Glyceraldehyde-3-phosphate dehydrogenase - P04406
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 33 - protein, 417 aa, Homo sapiens
  source 1..417: note = Phosphoglycerate kinase 1 P00558
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI

SEQ ID NO: 34 - protein, 417 aa, Homo sapiens
  source 1..417: note = Phosphoglycerate kinase 2 - P07205
MSLSKKLTLDKLDVRGKRVIMRVDFNVPMKKNQITNNQRIKASIPSIKYCLDNGAKAVVLMSHLGRPDGVPMPDKYSLAPVAVELKSLLGKDVLFLKDCVGAEVEKACANPAPGSVILLENLRFHVEEEGKGQDPSGKKIKAEPDKIEAFRASLSKLGDVYVNDAFGTAHRAHSSMVGVNLPHKASGFLMKKELDYFAKALENPVRPFLAILGGAKVADKIQLIKNMLDKVNEMIIGGGMAYTFLKVLNNMEIGASLFDEEGAKIVKDIMAKAQKNGVRITFPVDFVTGDKFDENAQVGKATVASGISPGWMGLDCGPESNKNHAQVVAQARLIVWNGPLGVFEWDAFAKGTKALMDEIVKATSKGCITVIGGGDTATCCAKWNTEDKVSHVSTGGGASLELLEGKILPGVEALSNM

SEQ ID NO: 35 - protein, 254 aa, Homo sapiens
  source 1..254: note = phosphoglycerate mutase 1 (brain) P18669
MAAYKLVLIRHGESAWNLENRFSGWYDADLSPAGHEEAKRGGQALRDAGYEFDICFTSVQKRAIRTLWTVLDAIDQMWLPVVRTWRLNERHYGGLTGLNKAETAAKHGEAQVKIWRRSYDVPPPPMEPDHPFYSNISKDRRYADLTEDQLPSCESLKDTIARALPFWNEEIVPQIKEGKRVLIAAHGNSLRGIVKHLEGLSEEAIMELNLPTGIPIVYELDKNLKPIKPMQFLGDEETVRKAMEAVAAQGKAKK

SEQ ID NO: 36 - protein, 253 aa, Homo sapiens
  source 1..253: note = phosphoglycerate mutase 1 (muscle) P15259
MATHRLVMVRHGESTWNQENRFCGWFDAELSEKGTEEAKRGAKAIKDAKMEFDICYTSVLKRAIRTLWAILDGTDQMWLPVVRTWRLNERHYGGLTGLNKAETAAKHGEEQVKIWRRSFDIPPPPMDEKHPYYNSISKERRYAGLKPGELPTCESLKDTIARALPFWNEEIVPQIKAGKRVLIAAHGNSLRGIVKHLEGMSDQAIMELNLPTGIPIVYELNKELKPTKPMQFLGDEETVRKAMEAVAAQGKAK

SEQ ID NO: 37 - protein, 434 aa, Homo sapiens
  source 1..434: note = phosphopyruvate hydratase - Q6FHV6
MSIEKIWAREILDSRGNPTVEVDLYTAKGLFRAAVPSGASTGIYEALELRDGDKQRYLGKGVLKAVDHINSTIAPALISSGLSVVEQEKLDNLMLELDGTENKSKFGANAILGVSLAVCKAGAAERELPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKIVIGMDVAASEFYRDGKYDLDFKSPTDPSRYITGDQLGALYQDFVRDYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 38 - protein, 531 aa, Homo sapiens
  source 1..531: note = Pyruvate Kinase P14618-2
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAMFHRKLFEELVRASSHSTDLMEAMAMGSVEASYKCLAAALIVLTESGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 39 - protein, 277 aa, synthetic construct [sequence omitted]

SEQ ID NO: 40 - DNA, 2984 bp, synthetic construct
  source 1..2984: mol_type = other DNA; note = Glut3 IRES NGFR
atggggacacagaaggtcaccccagctctgatatttgccatcacagttgctacaatcggctctttccaatttggctacaacactggggtcatcaatgctcctgagaagatcataaaggaatttatcaataaaactttgacggacaagggaaatgccccaccctctgaggtgctgctcacgtctctctggtccttgtctgtggccatattttccgtcgggggtatgatcggctccttttccgtcggactcttcgtcaaccgctttggcaggcgcaattcaatgctgattgtcaacctgttggctgtcactggtggctgctttatgggactgtgtaaagtagctaagtcggttgaaatgctgatcctgggtcgcttggttattggcctcttctgcggactctgcacaggttttgtgcccatgtacattggagagatctcgcctactgccctgcggggtgcctttggcactctcaaccagctgggcatcgttgttggaattctggtggcccagatctttggtctggaattcatccttgggtctgaagagctatggccgctgctactgggttttaccatccttcctgctatcctacaaagtgcagcccttccattttgccctgaaagtcccagatttttgctcattaacagaaaagaagaggagaatgctaagcagatcctccagcggttgtggggcacccaggatgtatcccaagacatccaggagatgaaagatgagagtgcaaggatgtcacaagaaaagcaagtcaccgtgctagagctctttagagtgtccagctaccgacagcccatcatcatttccattgtgctccagctctctcagcagctctctgggatcaatgctgtgttctattactcaacaggaatcttcaaggatgcaggtgttcaagagcccatctatgccaccatcggcgcgggtgtggttaatactatcttcactgtagtttctctatttctggtggaaagggcaggaagaaggactctgcatatgataggccttggagggatggctttttgttccacgctcatgactgtttctttgttattaaaggataactataatgggatgagctttgtctgtattggggctatcttggtctttgtagccttctttgaaattggaccaggccccattccctggtttattgtggccgaactcttcagccagggcccccgcccagctgcgatggcagtggccggctgctccaactggacctccaacttcctagtcggattgctcttcccctccgctgctcactatttaggagcctacgtttttattatcttcaccggcttcctcattaccttcttggcttttaccttcttcaaagtccctgagacccgtggcaggacttttgaggatatcacacgggcctttgaagggcaggcacacggtgcagatagatctggaaaggacggcgtcatggagatgaacagcatcgagcctgctaaggagaccaccaccaatgtttaagcggccgctctagaactagtggatctccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatcaagcttatcgatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgcccagacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgatgaattctgcagtcgacggtaccgcgg

SEQ ID NO: 41 - DNA, 3839 bp, synthetic construct
  source 1..3839: mol_type = other DNA; note = PFK IRES NGFR
atgtcgacccatgaagagcaccatgcagccaaaaccctggggattggcaaagccattgctgtcttaacctctggtggagatgcccaaggtatgaatgctgctgtcagggctgtggttcgagttggtatcttcaccggtgcccgtgtcttctttgtccatgagggttatcaaggcctggtggatggtggagatcacatcaaggaagccacctgggagagcgtttcgatgatgcttcagctgggaggcacggtgattggaagtgcccggtgcaaggactttcgggaacgagaaggacgactccgagctgcctacaacctggtgaagcgtgggatcaccaatctctgtgtcattgggggtgatggcagcctcactggggctgacaccttccgttctgagtggagtgacttgttgagtgacctccagaaagcaggtaagatcacagatgaggaggctacgaagtccagctacctgaacattgtgggcctggttgggtcaattgacaatgacttctgtggcactgatatgaccattggcactgactctgccctgcatcggatcatggaaattgtagatgccatcactaccactgcccagagccaccagaggacatttgtgttagaagtaatgggccgccactgtggatacctggcccttgtcacctctctgtcctgtggggccgactgggtttttattcctgaatgtccaccagatgacgactgggaggaacacctttgtcgccgactcagcgagacaaggacccgtggttctcgtctcaacatcatcattgtggctgagggtgcaattgacaagaatggaaaaccaatcacctcagaagacatcaagaatctggtggttaagcgtctgggatatgacacccgggttactgtcttggggcatgtgcagaggggtgggacgccatcagcctttgacagaattctgggcagcaggatgggtgtggaagcagtgatggcacttttggaggggaccccagataccccagcctgtgtagtgagcctctctggtaaccaggctgtgcgcctgcccctcatggaatgtgtccaggtgaccaaagatgtgaccaaggccatggatgagaagaaatttgacgaagccctgaagctgagaggccggagcttcatgaacaactgggaggtgtacaagcttctagctcatgtcagacccccggtatctaagagtggttcgcacacagtggctgtgatgaacgtgggggctccggctgcaggcatgaatgctgctgttcgctccactgtgaggattggccttatccagggcaaccgagtgctcgttgtccatgatggtttcgagggcctggccaaggggcagatagaggaagctggctggagctatgttgggggctggactggccaaggtggctctaaacttgggactaaaaggactctacccaagaagagctttgaacagatcagtgccaatataactaagtttaacattcagggccttgtcatcattgggggctttgaggcttacacagggggcctggaactgatggagggcaggaagcagtttgatgagctctgcatcccatttgtggtcattcctgctacagtctccaacaatgtccctggctcagacttcagcgttggggctgacacagcactcaatactatctgcacaacctgtgaccgcatcaagcagtcagcagctggcaccaagcgtcgggtgtttatcattgagactatgggtggctactgtggctacctggctaccatggctggactggcagctggggccgatgctgcctacatttttgaggagcccttcaccattcgagacctgcaggcaaatgttgaacatctggtgcaaaagatgaaaacaactgtgaaaaggggcttggtgttaaggaatgaaaagtgcaatgagaactataccactgacttcattttcaacctgtactctgaggaggggaagggcatcttcgacagcaggaagaatgtgcttggtcacatgcagcagggtgggagcccaaccccatttgataggaattttgccactaagatgggcgccaaggctatgaactggatgtctgggaaaatcaaagagagttaccgtaatgggcggatctttgccaatactccagattcgggctgtgttctggggatgcgtaagagggctctggtcttccaaccagtggctgagctgaaggaccagacagattttgagcatcgaatccccaaggaacagtggtggctgaaactgaggcccatcctcaaaatcctagccaagtacgagattgacttggacacttcagaccatgcccacctggagcacatcacccggaagcggtccggggaagctgccgtctaagcggccgctctagaactagtggatctccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatcaagcttatcgatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgcccagacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgatgaattctgcagtcgacggtaccgcgg